(19) United States
(12) Patent Application Publication
(10) Pub. No.: US 2015/0376196 A1
(43) Pub. Date: Dec. 31, 2015

(54) 4-SUBSTITUTED PYRROLO- AND PYRAZOLO-DIAZEPINES

(71) Applicant: BAYER PHARMA AKTIENGESELLSCHAFT, Berlin (DE)

(72) Inventors: Norbert SCHMEES, Berlin (DE); Bernd BUCHMANN, Hohen Neuendorf (DE); Bernard HAENDLER, Berlin (DE); Roland NEUHAUS, Berlin (DE); Pascale LEJEUNE, Berlin (DE); Martin KRÜGER, Berlin (DE); Amaury Ernesto FERNANDEZ-MONTALVAN, Berlin (DE); Hermann KÜNZER, (US); Hartmut REHWINKEL, Berlin (DE)

(21) Appl. No.: 14/769,921

(22) PCT Filed: Feb. 18, 2014
(86) PCT No.: PCT/EP2014/053099

§ 371 Date: Aug. 24, 2015

(30) Foreign Application Priority Data
Feb. 22, 2013 (DE) 10 2013 202 991.8

(51) Int. Cl. C07D 487/14; C07D 519/00; A61K 31/5517

(52) U.S. Cl. CPC C07D 487/14; A61K 31/5517; C07D 519/00

(57) ABSTRACT

BET protein-inhibitory, especially BRD2-, BRD3- and BRD4-inhibitory, 4-substituted pyrrolo- and pyrazolodiazepines of the general formula I

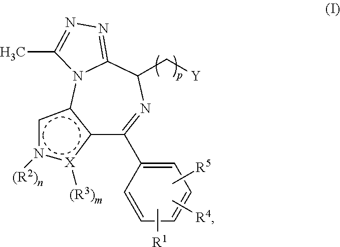

are described, in which X, Y, n, m, p, R¹, R², R³, R⁴ and R⁵ are each as defined in the description, as are pharmaceutical compositions comprising the inventive compounds, and the prophylactic and therapeutic use thereof in the case of hyperproliferative disorders, especially in the case of neoplastic disorders. Also described is the use of the inventive compounds as BET protein inhibitors in benign hyperplasias, in atherosclerotic disorders, in sepsis, in autoimmune disorders, in vascular disorders, in viral infections, in neurodegenerative disorders, in inflammatory disorders and in male fertility control.

The present invention relates to BET protein-inhibitory, especially BRD2-, BRD3- and BRD4-inhibitory, 4-substituted pyrrolo- and pyrazolodiazepines, to pharmaceutical compositions comprising the inventive compounds, and to the prophylactic and therapeutic use thereof in the case of hyperproliferative disorders, especially in the case of neoplastic disorders. The present invention further relates to the use of BET protein inhibitors in benign hyperplasias, in atherosclerotic disorders, in sepsis, in autoimmune disorders, in vascular disorders, in viral infections, in neurodegenerative disorders, in inflammatory disorders, in atherosclerotic disorders and in male fertility control.

The human BET family (bromo domain and extra C-terminal domain family) has four members (BRD2, BRD3, BRD4 and BRDT) containing two related bromo domains and one extraterminal domain (Wu and Chiang, J. Biol. Chem., 2007 (282), 13141-13145). The bromo domains are protein regions which recognize acetylated lysine residues. Such acetylated lysines are often found at the N-terminal end of histones (e.g. histone H3 or histone H4), and they are features of an open chromatin structure and active gene transcription (Kuo and Allis, Bioessays, 1998, 20:615-626). The various acetylation patterns which are recognized by BET proteins in histones have been studied in detail (Umehara et al., J. Biol. Chem., 2010, 285:7610-7618; Filippakopoulos et al., Cell, 2012, 149:214-231). In addition, bromo domains can recognize further acetylated proteins. For example, BRD4 binds to RelA, which leads to stimulation of NF-κB and transcriptional activity of inflammatory genes (Huang et al., Mol. Cell. Biol., 2009, 29:1375-1387; Zhang et al., J. Biol. Chem., 2012, doi/10.1074/jbc.M112.359505). The extraterminal domain of BRD2, BRD3 and BRD4 interacts with several proteins involved in chromatin modulation and the regulation of gene expression (Rahman et al., Mol. Cell. Biol., 2011, 31:2641-2652).

In mechanistic terms, BET proteins play an important role in cell growth and in the cell cycle. They are associated with mitotic chromosomes, which suggests a function in epigenetic memory (Dey et al., Mol. Biol. Cell, 2009, 20:4899-4909; Yang et al., Mol. Cell. Biol., 2008, 28:967-976). BRD4 is important for post-mitotic reactivation of gene transcription (Zhao et al., Nat. Cell. Biol., 2011, 13:1295-1304). It has been shown that BRD4 is essential for transcription elongation and for the recruitment of the elongation complex P-TEFb consisting of CDK9 and cyclin T1, which leads to activation of RNA polymerase II (Yang et al., Mol. Cell, 2005, 19:535-545; Schroder et al., J. Biol. Chem., 2012, 287:1090-1099). Consequently, the expression of genes involved in cell proliferation is stimulated, for example of c-Myc and Aurora B (You et al., Mol. Cell. Biol., 2009, 29:5094-5103; Zuber et al., Nature, 2011, 478:524-528). BRD2 and BRD3 bind to transcribed genes in hyperacetylated chromatin regions and promote transcription by RNA polymerase II (LeRoy et al., Mol. Cell, 2008, 30:51-60).

The knockdown of BRD4 or the inhibition of the interaction with acetylated histones in various cell lines leads to a G1 arrest and to cell death by apoptosis (Mochizuki et al., J. Biol. Chem., 2008, 283:9040-9048; Mertz et al., Proc. Natl. Acad. Sci. USA, 2011, 108:16669-16674). It has also been shown that BRD4 binds to promoter regions of several genes which are activated in the G1 phase, for example cyclin D1 and D2 (Mochizuki et al., J. Biol. Chem., 2008, 283:9040-9048). In addition, inhibition of the expression of c-Myc, an essential factor in cell proliferation, after BRD4 inhibition has been demonstrated (Dawson et al., Nature, 2011, 478:529-533; Delmore et al., Cell, 2011, 146:1-14; Mertz et al., Proc. Natl. Acad. Sci. USA, 2011, 108:16669-16674).

BRD2 and BRD4 knockout mice die at an early stage during embryogenesis (Gyuris et al., Biochim Biophys. Acta, 2009, 1789:413-421; Houzelstein et al., Mol. Cell. Biol., 2002, 22:3794-3802). Heterozygotic BRD4 mice have various growth defects attributable to reduced cell proliferation (Houzelstein et al., Mol. Cell. Biol., 2002, 22:3794-3802).

BET proteins play an important role in various tumour types. Fusion between the BET proteins BRD3 or BRD4 and NUT, a protein which is normally expressed only in the testes, leads to an aggressive form of squamous cell carcinoma, called NUT midline carcinoma (French, Cancer Genet. Cytogenet., 2010, 203:16-20). The fusion protein prevents cell differentiation and promotes proliferation (Yan et al., J. Biol. Chem., 2011, 286:27663-27675). The growth of in vivo models derived therefrom is inhibited by a BRD4 inhibitor (Filippakopoulos et al., Nature, 2010, 468:1067-1073). Screening for therapeutic targets in an acute myeloid leukaemia cell line (AML) showed that BRD4 plays an important role in this tumour (Zuber et al., Nature, 2011, doi:10.1038). Reduction in BRD4 expression leads to a selective arrest of the cell cycle and to apoptosis. Treatment with a BRD4 inhibitor prevents the proliferation of an AML xenograft in vivo. Amplification of the DNA region containing the BRD4 gene was detected in primary breast tumours (Kadota et al., Cancer Res, 2009, 69:7357-7365). For BRD2 too, there are data relating to a role in tumours. A transgenic mouse which overexpresses BRD2 selectively in B cells develops B cell lymphomas and leukaemias (Greenwall et al., Blood, 2005, 103:1475-1484).

BET proteins are also involved in viral infections. BRD4 binds to the E2 protein of various papillomaviruses and is important for the survival of the viruses in latently infected cells (Wu et al., Genes Dev., 2006, 20:2383-2396; Vosa et al., J. Virol., 2012, 86:348-357). The herpes virus, which is responsible for Kaposi's sarcoma, also interacts with various BET proteins, which is important for disease survival (Viejo-Borbolla et al., J. Virol., 2005, 79:13618-13629; You et al., J. Virol., 2006, 80:8909-8919). Through binding to P-TEFb, BRD4 also plays an important role in the replication of HIV (Bisgrove et al., Proc. Natl Acad. Sci. USA, 2007, 104:13690-13695).

BET proteins are additionally involved in inflammation processes. BRD2-hypomorphic mice show reduced inflammation in adipose tissue (Wang et al., Biochem. J., 2009, 425:71-83). Infiltration of macrophages in white adipose tissue is also reduced in BRD2-deficient mice (Wang et al., Biochem. J., 2009, 425:71-83). It has also been shown that BRD4 regulates a number of genes involved in inflammation. In LPS-stimulated macrophages, a BRD4 inhibitor prevents the expression of inflammatory genes, for example IL-1 or IL-6 (Nicodeme et al., Nature, 2010, 468:1119-1123). BET proteins also regulate the expression of the ApoAl gene, which plays an important role in atherosclerosis and inflammatory processes (Chung et al., J. Med. Chem, 2011, 54:3827-3838). Apolipoprotein A1 (ApoAl) is a major component of high density lipoproteins (HDL), and increased expression of ApoAl leads to elevated blood cholesterol values (Degoma and Rader, Nat. Rev. Cardiol., 2011, 8:266-277). Elevated HDL values are associated with a reduced risk of atherosclerosis (Chapman et al., Eur. Heart J., 2011, 32:1345-1361).

### PRIOR ART

The nomenclature employed in the assessment of the structural prior art is illustrated by the following figure:

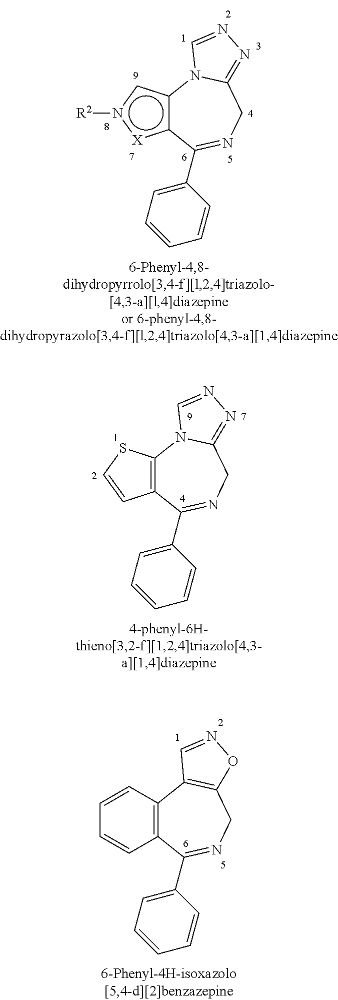

Based on the chemical structure, some types of BRD4 inhibitors have been described to date (Chun-Wa Chung et al., Progress in Medicinal Chemistry 2012, 51, 1-55).

The first published BRD4 inhibitors are phenylthienotriazolo-1,4-diazepines (4-phenyl-6H-thieno[3,2-f][1,2,4]triazolo[4,3-a][1,4]diazepines) as described in WO2009/084693 (Mitsubishi Tanabe Pharma Corporation) and as compound JQ1 in WO2011/143669 (Dana Farber Cancer Institute). Replacement of the thieno moiety by a benzo moiety also leads to active inhibitors (J. Med. Chem. 2011, 54, 3827 3838; E. Nicodeme et al., Nature 2010, 468, 1119). These and one further publication show that the pyrazole unit fused to the 1,4-benzodiazepine or thieno-1,4-diazepine ring system is actively involved in binding of the target protein BRD4 (P. Filippakopoulos et al., Nature 2010, 468, 1067). Further 4-phenyl-6H-thieno[3,2-f][1,2,4]triazolo[4,3-a][1,4]diazepines and related compounds having alternative rings as a fusion partner rather than the benzo unit are addressed generically or described directly in WO2012/075456 (Constellation Pharmaceuticals). WO2012/075383 (Constellation Pharmaceuticals) describes 6-substituted 4H-isoxazolo[5,4-d][2]benzazepines and 4H-isoxazolo[3,4-d][2]benzazepines, including compounds which have optionally substituted phenyl at position 6 as BRD4 inhibitors, and also analogues with alternative heterocyclic fusion partners rather than the benzo unit, for example thieno- or pyridoazepines.

WO2013/184876 and WO2013/184878 (Constellation Pharmaceuticals) describe further benzoisoxazoloazepine derivatives as inhibitors of proteins comprising bromo domains. Another structural class of BRD4 inhibitors described is that of 7-isoxazoloquinolines and related quinoline derivatives (WO2011/054843, Bioorganic & Medicinal Chemistry Letters 22 (2012) 2963-2967, GlaxoSmithKline). Pyridinones and pyridazinones (WO 2013/185284, WO 2013/188381; Abbott Laboratories) and also isoindolones (WO 2013/155695, WO 2013/158952; Abbott Laboratories) have been described as inhibitors of binding of the bromo domains of the BET proteins to proteins comprising N-acetylated lysine residues.

WO94/26718/EP0703222A1 (Yoshitomi Pharmaceutical Industries) describes substituted 3-amino-2,3-dihydro-1H-1-benzazepin-2-ones or the corresponding 2-thiones and analogues in which the benzo unit has been replaced by alternative monocyclic systems, and in which the 2-ketone or the 2-thione together with the substituted nitrogen atom in the azepine ring may form a heterocycle, as CCK and gastrin antagonists for the treatment of CNS disorders, such as states of anxiety and depression, and of pancreatic disorders and of gastrointestinal ulcers. Ligands of the gastrin and the cholecystokinin receptor are described in WO2006/051312 (James Black Foundation). They also include substituted 3,5-dihydro-4H-2,3-benzodiazepin-4-ones which differ from the inventive compounds mainly by the obligatory oxo group in position 4 and by an obligatory carbonyl group-containing alkyl chain in position 5. Finally, substituted 3,5-dihydro-4H-2,3-benzodiazepin-4-ones are also described as AMPA antagonists in WO97/34878 (Cocensys Inc.). The generic claim is very wide with respect to the possible substitution patterns at the benzodiazepine skeleton; however, the working examples are limited to a narrow range. EP102602 furthermore describes 6-aryldiazepinones having a fused pyrrole ring which are used as spasmolytics and for anxiety. These may carry side chains in position 4 which are attached via an oxygen or nitrogen atom. Attachment via a carbon atom has hitherto not been described.

DE3435973 describes 6-aryltriazolodiazepines which carry a fused pyrrole ring having a nitrogen in position 2. The compounds are used for treating pathological states and diseases where acetyl glyceryl ether phosphorylcholine (PAF) is involved. However, these compounds do not have a side chain in position 4. Only substitution by a methyl group has been described at a diazepinone system having fused pyrazole (J. Med. Chem. 24, (1981), pp. 982 ff., DeWald et al.).

Furthermore, fusion of pyrazole to the nitrogen atoms in positions 2 and 3 is described in U.S. Pat. No. 3,657,271. However, these compounds do not carry a further fused triazole ring and no side chain in position 4 either. The compounds demonstrate anti-inflammatory activity.

It would therefore be desirable to provide novel compounds having improved prophylactic and therapeutic properties.

Accordingly, it is an object of the present invention to provide compounds and pharmaceutical compositions comprising these compounds which are employed for prophylactic and therapeutic applications for hyperproliferative disorders, in particular for neoplastic disorders, and as BET protein inhibitors for viral infections, for benign hyperplasias, for neurodegenerative disorders, for inflammatory disorders, for atherosclerotic disorders, for autoimmune disorders, for vascular disorders, for sepsis and for male fertility control.

The inventive compounds are novel 4-substituted pyrrolo- and pyrazolodiazepines which, surprisingly, have BET protein-inhibitory, especially BRD2-, BRD3- and BRD4-inhibitory, activity, and which inhibit interaction between BRD4 inhibitors and an acetylated histone H4 peptide and inhibit the growth of cancer cells.

From the prior art described above, there was no reason to modify the structures of the prior art such that structures suitable for the prophylaxis and therapy of neoplastic disorders are obtained.

Surprisingly, the inventive compounds inhibit the interaction between BRD4 and an acetylated histone H4 peptide and inhibit the growth of cancer cells. Accordingly, they provide novel structures for the therapy of human and animal disorders, in particular of cancers.

It has now been found that compounds of the general formula I

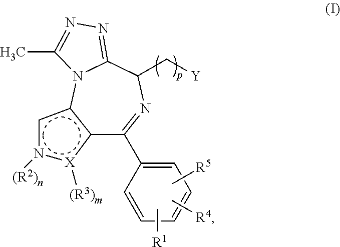

in which
X is a carbon or nitrogen atom,
n and m are each independently 0 or 1,
P is 1, 2, 3 or 4,
R¹, R⁴ and R⁵ are each independently hydrogen, hydroxyl, cyano, nitro, amino, aminocarbonyl, halogen or optionally singly or multiply, identically or differently halogen-, amino-, hydroxyl-, carboxyl-, hydroxy-C₁-C₆-alkyl-, C₁-C₆-alkoxy-, C₁-C₆-alkoxy-C₁-C₆-alkyl-, C₁-C₆-alkylamino- or amino-C₁-C₆-alkyl-substituted C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl,
R² is hydrogen or is C₁-C₆-alkyl, aminocarbonyl, C₁-C₆-alkylcarbonyl, C₁-C₆-alkylaminocarbonyl, C₁-C₆-alkylsulphonyl, phenylsulphonyl or C₁-C₆-alkylaminosulphonyl, each optionally mono- or polysubstituted identically or differently by halogen, amino, hydroxyl, carboxyl, hydroxy-C₁-C₆-alkyl, C₁-C₆-alkoxy, C₁-C₆-alkoxy-C₁-C₆-alkyl, C₁-C₆-alkylamino, amino-C₁-C₆-alkyl, monocyclic heterocyclyl having 4 to 8 ring atoms or monocyclic heteroaryl having 5 or 6 ring atoms, in which the monocyclic heterocyclyl and heteroaryl are optionally monosubstituted by C₁-C₃-alkyl,
   or
   is C₃-C₁₀-cycloalkyl optionally mono- or polysubstituted identically or differently by halogen, amino, hydroxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy or a monocyclic heterocyclyl radical having 4 to 8 ring atoms, or
   is monocyclic heteroaryl which has 5 or 6 ring atoms and is optionally mono- or polysubstituted identically or differently by halogen, amino, hydroxyl, cyano, nitro, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₁-C₆-alkylcarbonyl, C₁-C₆-alkylsulphonyl, C₃-C₁₀-cycloalkyl or a monocyclic heterocyclyl radical having 4 to 8 ring atoms,
   or
   is monocyclic heterocyclyl which has 4 to 8 ring atoms and is optionally mono- or polysubstituted identically or differently by halogen, amino, hydroxyl, cyano, oxo, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylamino-C₁-C₆-alkyl, hydroxy-C₁-C₆-alkyl, halo-C₁-C₆-alkyl, halo-C₁-C₆-alkoxy, C₁-C₆-alkylcarbonyl, C₁-C₆-alkylsulphonyl, C₃-C₁₀-cycloalkyl or a monocyclic heterocyclyl radical having 4 to 8 ring atoms,
   or
   is phenyl optionally mono- or polysubstituted identically or differently by halogen, amino, hydroxyl, cyano, nitro, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylaminocarbonyl, C₁-C₆-alkylaminosulphonyl, C₁-C₆-alkylamino-C₁-C₆-alkyl, hydroxy-C₁-C₆-alkyl, halo-C₁-C₆-alkyl, halo-C₁-C₆-alkoxy, C₁-C₆-alkylcarbonyl, C₁-C₆-alkylsulphonyl, C₃-C₁₀-cycloalkyl or a monocyclic heterocyclyl radical having 4 to 8 ring atoms,
   or
R³ is hydrogen, hydroxyl, cyano, nitro, amino, aminocarbonyl, halogen or is C₁-C₆-alkyl, C₁-C₆-alkoxy, C₁-C₆-alkylcarbonyl, C₁-C₆-alkylsulphonyl, phenylsulphonyl, C₁-C₆-alkylamino, C₁-C₆-alkylcarbonylamino, C₁-C₆-alkylaminocarbonyl or C₁-C₆-alkylaminosulphonyl, each optionally mono- or polysubstituted identically or differently by halogen, amino, hydroxyl, carboxyl, hydroxy-C₁-C₆-alkyl, C₁-C₆-alkoxy, C₁-C₆-alkoxy-C₁-C₆-alkyl, C₁-C₆-alkylamino or amino-C₁-C₆-alkyl, when X is a carbon atom,
   or
R³ is hydrogen or is C₁-C₆-alkyl, C₁-C₆-alkoxy, C₁-C₆-alkylcarbonyl, C₁-C₆-alkylaminocarbonyl, C₁-C₆-alkylsulphonyl, phenylsulphonyl or C₁-C₆-alkylaminosulphonyl, each optionally mono- or polysubstituted identically or differently by halogen, amino, hydroxyl, carboxyl, hydroxy-C₁-C₆-alkyl, C₁-C₆-alkoxy, C₁-C₆-alkoxy-C₁-C₆-alkyl, C₁-C₆-alkylamino or amino-C₁-C₆-alkyl, when X is a nitrogen atom,
   or
R² and R³ together with the ring atoms N and X may form a further heteroaromatic or heterocyclic ring which has 5 to 7 ring atoms and may optionally be mono- or polysubstituted identically or differently by halogen, amino, hydroxyl, cyano, nitro, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylaminocarbonyl, C₁-C₆-alkylaminosulphonyl, C₁-C₆-alkylcarbonyl, C₁-C₆-alkylsulphonyl, C₁-C₆-alkylamino-C₁-C₆-alkyl, hydroxy-C₁-C₆-alkyl, halo-C₁-C₆-alkyl, halo-C₁-C₆-alkoxy, C₃-C₁₀-cycloalkyl or a monocyclic heterocyclyl radical having 4 to 8 ring atoms,
Y is a —C(═O)OR¹², —C(═O)R¹³ or —C(═O)NR¹⁰R¹¹ group, or is phenyl, monocyclic heterocyclyl having 4 to 8 ring atoms or mono- or bicyclic heteroaryl having 5 to 10 ring atoms, in which phenyl, heteroaryl and heterocyclyl are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, phenoxy, halophenyl, phenyl-C₁-C₆-alkyl, phenyl-C₁-C₆-alkoxy, heteroaryl having 5 or 6 ring atoms,
   —C(═O)—NR6R7, —C(═O)—R8, —S(═O)₂—NR6R7, —S(═O)—R9, —S(═O)₂—R9, —NH—S(═O)₂—R⁹, or a monocyclic heterocyclyl radical which has 4 to 8 ring atoms and is itself optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl or halo-C₁-C₆-alkoxy,
R⁶ and R⁷ are each independently hydrogen, C₁-C₃-alkyl, C₃-C₇-cycloalkyl or di-C₁-C₃-alkylamino-C₁-C₃-alkyl, each optionally mono- or polysubstituted identically or differently by halogen, cyano, amino or oxo,
R⁸ is hydroxyl, C₁-C₆-alkyl, C₁-C₆-alkoxy, halo-C₁-C₃-alkyl, hydroxy-C₁-C₃-alkyl, C₁-C₃-alkoxy-C₁-C₃-alkyl, C₃-C₇-cycloalkyl, phenyl, monocyclic heterocyclyl having 4 to 8 ring atoms or monocyclic heteroaryl having 5 or 6 ring atoms, where phenyl, heteroaryl and heterocyclyl are optionally mono- or disubstituted by halogen, C₁-C₃-alkoxy or C₁-C₃-alkyl,
R⁹ is C₁-C₆-alkyl or C₃-C₇-cycloalkyl,
R¹⁰ and R¹¹ are each independently hydrogen, C₁-C₆-alkyl or a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, C₁-C₆-alkylcarbonylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, phenoxy, monocyclic heteroaryl having 5 or 6 ring atoms, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ or a monocyclic heterocyclyl radical having 4 to 8 ring atoms,
   or
R¹⁰ and R¹¹ together with the adjacent nitrogen atom are a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, C₁-C₆-alkylcarbonylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, phenoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ or a monocyclic heterocyclyl radical having 4 to 8 ring atoms,
R₁₂ is C₁-C₆-alkyl, halo-C₁-C₃-alkyl, hydroxy-C₁-C₃-alkyl, C₁-C₃-alkoxy-C₁-C₃-alkyl, C₃-C₈-cycloalkyl, phenyl, a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, C₁-C₆-alkylcarbonylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, phenoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ or a monocyclic heterocyclyl radical having 4 to 8 ring atoms,
R¹³ is C₁-C₆-alkyl, halo-C₁-C₃-alkyl, hydroxy-C₁-C₃-alkyl, C₁-C₃-alkoxy-C₁-C₃-alkyl, C₃-C₈-cycloalkyl, phenyl, a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, C₁-C₆-alkylcarbonylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, phenoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ or a monocyclic heterocyclyl radical having 4 to 8 ring atoms,
   and their polymorphs, enantiomers, diastereomers, racemates, tautomers, solvates, physiologically acceptable salts and solvates of these salts are particularly suitable for a large number of prophylactic and therapeutic applications, in particular for hyperproliferative disorders, for neoplastic disorders and as BET protein inhibitors, for benign hyperplasias, atherosclerotic disorders, sepsis, autoimmune disorders, vascular disorders, viral infections, for neurodegenerative disorders, for inflammatory disorders and for male fertility control.

Of particular interest are those compounds of the general formula I in which
X is a carbon or nitrogen atom,
n and m are each independently 0 or 1,
p is 1,
R¹, R⁴ and R⁵ are each independently hydrogen, hydroxyl, cyano, nitro, amino, aminocarbonyl, halogen or optionally singly or multiply, identically or differently halogen-, amino-, hydroxyl-, carboxyl-, hydroxy-C₁-C₆-alkyl-, C₁-C₆-alkoxy-, C₁-C₆-alkoxy-C₁-C₆-alkyl-, C₁-C₆-alkylamino- or amino-C₁-C₆-alkyl-substituted C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl,
R² is hydrogen or is C₁-C₆-alkyl, C₁-C₆-alkylcarbonyl, phenylsulphonyl or C₁-C₆-alkylsulphonyl, each optionally mono- or polysubstituted identically or differently by halogen, amino, hydroxyl, carboxyl, hydroxy-C₁-C₆-alkyl, C₁-C₆-alkoxy, C₁-C₆-alkoxy-C₁-C₆-alkyl, C₁-C₆-alkylamino, amino-C₁-C₆-alkyl, monocyclic heterocyclyl having 4 to 8 ring atoms or monocyclic heteroaryl having 5 or 6 ring atoms, in which the monocyclic heterocyclyl and heteroaryl are optionally monosubstituted by C₁-C₃-alkyl, or
   is C₃-C₁₀-cycloalkyl which is optionally mono- or polysubstituted identically or differently by halogen, amino, hydroxyl, C₁-C₆-alkyl, C₁-C₆-alkoxy, C₁-C₆-alkoxy-C₁-C₆-alkyl, C₁-C₆-alkylamino-, amino-C₁-C₆-alkyl, C₁-C₆-alkylamino-C₁-C₆-alkyl, halo-C₁-C₆-alkyl, halo-C₁-C₆-alkoxy or a monocyclic heterocyclyl radical having 4 to 8 ring atoms,
   or
   is monocyclic heteroaryl which has 5 or 6 ring atoms and is optionally mono- or polysubstituted identically or differently by halogen, amino, hydroxyl, cyano, nitro, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₁-C₆-alkylcarbonyl, C₁-C₆-alkylsulphonyl, C₃-C₁₀-cycloalkyl or a monocyclic heterocyclyl radical having 4 to 8 ring atoms,
   or
   is monocyclic heterocyclyl which has 4 to 8 ring atoms and is optionally mono- or polysubstituted identically or differently by halogen, amino, hydroxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₁-C₆-alkylsulphonyl or a monocyclic heterocyclyl radical having 4 to 8 ring atoms,
   or
   is phenyl optionally mono- or polysubstituted identically or differently by halogen, amino, hydroxyl, cyano, nitro, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylaminocarbonyl, C₁-C₆-alkylaminosulphonyl, C₁-C₆-alkylamino-C₁-C₆-alkyl, hydroxy-C₁-C₆-alkyl, halo-C₁-C₆-alkyl, halo-C₁-C₆-alkoxy, C₁-C₆-alkylsulphonyl, C₃-C₁₀-cycloalkyl or a monocyclic heterocyclyl radical having 4 to 8 ring atoms,
R³ is hydrogen or is C₁-C₆-alkyl, C₁-C₆-alkylcarbonyl, C₁-C₆-alkylsulphonyl or phenylsulphonyl, each optionally mono- or polysubstituted identically or differently by halogen, amino, hydroxyl, carboxyl, hydroxy-C₁-C₆-alkyl, C₁-C₆-alkoxy, C₁-C₆-alkoxy-C₁-C₆-alkyl, C₁-C₆-alkylamino or amino-C₁-C₆-alkyl, when X is a carbon atom,
   or
R³ is hydrogen or is C₁-C₆-alkyl, C₁-C₆-alkylcarbonyl, C₁-C₆-alkylsulphonyl or phenylsulphonyl, each optionally mono- or polysubstituted identically or differently by halogen, amino, hydroxyl, carboxyl, hydroxy-C₁-C₆-alkyl, C₁-C₆-alkoxy, C₁-C₆-alkoxy-C₁-C₆-alkyl, C₁-C₆-alkylamino or amino-C₁-C₆-alkyl, when X is a nitrogen atom,
   or
R² and R³ together with the ring atoms N and X may form a further heteroaromatic or heterocyclic ring which has 5 to 7 ring atoms and is optionally mono- or polysubstituted identically or differently by halogen, amino, hydroxyl, cyano, nitro, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylaminocarbonyl, C₁-C₆-alkylaminosulphonyl, C₁-C₆-alkylcarbonyl, C₁-C₆-alkylsulphonyl, C₁-C₆-alkylamino-C₁-C₆-alkyl, hydroxy-C₁-C₆-alkyl, halo-C₁-C₆-alkyl, halo-C₁-C₆-alkoxy, C₃-C₁₀-cycloalkyl or a monocyclic heterocyclyl radical having 4 to 8 ring atoms,
Y is a —C(═O)OR¹², —C(═O)R¹³ or —C(═O)NR¹⁰R¹¹ group, or is phenyl, monocyclic heterocyclyl having 4 to 8 ring atoms or mono- or bicyclic heteroaryl having 5 to 10 ring atoms, in which phenyl, heteroaryl and heterocyclyl are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, phenoxy, halophenyl, phenyl-C₁-C₆-alkyl, phenyl-C₁-C₆-alkoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, or a monocyclic heterocyclyl radical which has 4 to 8 ring atoms and is itself optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl- or halo-C₁-C₆-alkoxy,
R⁶ and R⁷ are each independently hydrogen, C₁-C₃-alkyl, cyclopropyl or di-C₁-C₃-alkylamino-C₁-C₃-alkyl,
R⁸ is hydroxyl, C₁-C₆-alkyl, C₁-C₆-alkoxy, halo-C₁-C₃-alkyl, hydroxy-C₁-C₃-alkyl, C₁-C₃-alkoxy-C₁-C₃-alkyl, C₃-C₈-cycloalkyl, phenyl, monocyclic heterocyclyl having 4 to 8 ring atoms or monocyclic heteroaryl having 5 or 6 ring atoms, where phenyl, heteroaryl and heterocyclyl are optionally mono- or disubstituted by halogen, C₁-C₃-alkoxy or C₁-C₃-alkyl,
R⁹ is C₁-C₆-alkyl,
R¹⁰ and R¹¹ are each independently hydrogen, C₁-C₆-alkyl or a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, C₁-C₆-alkylcarbonylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, phenoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ or a monocyclic heterocyclyl radical having 4 to 8 ring atoms,
   or
R¹⁰ and R¹¹ together with the adjacent nitrogen atom are a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, C₁-C₆-alkylcarbonylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, phenoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ or a monocyclic heterocyclyl radical having 4 to 8 ring atoms,
R₁₂ is C₁-C₆-alkyl, halo-C₁-C₃-alkyl, hydroxy-C₁-C₃-alkyl, C₁-C₃-alkoxy-C₁-C₃-alkyl, C₃-C₈-cycloalkyl, phenyl, a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, C₁-C₆-alkylcarbonylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, phenoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ or a monocyclic heterocyclyl radical having 4 to 8 ring atoms,
R¹³ is C₁-C₆-alkyl, halo-C₁-C₃-alkyl, hydroxy-C₁-C₃-alkyl, C₁-C₃-alkoxy-C₁-C₃-alkyl, C₃-C₈-cycloalkyl, phenyl, a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, C₁-C₆-alkylcarbonylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, phenoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ or a monocyclic heterocyclyl radical having 4 to 8 ring atoms,
   and their polymorphs, enantiomers, diastereomers, racemates, tautomers, solvates, physiologically acceptable salts and solvates of these salts.

Of especial interest are those compounds of the general formula I in which
X is a carbon or nitrogen atom,
n and m are each independently 0 or 1,
p is 1,
R¹, R⁴ and R⁵ are each independently hydrogen, hydroxyl, cyano, aminocarbonyl, halogen or optionally singly or multiply, identically or differently halogen-, amino-, hydroxyl-, carboxyl-, hydroxy-C₁-C₆-alkyl-, C₁-C₆-alkoxy-, C₁-C₆-alkoxy-C₁-C₆-alkyl-, C₁-C₆-alkylamino- or amino-C₁-C₆-alkyl-substituted C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl,
R² is hydrogen or is C₁-C₆-alkyl, C₁-C₆-alkylcarbonyl, phenylsulphonyl or C₁-C₆-alkylsulphonyl, each optionally mono- or polysubstituted identically or differently by halogen, amino, hydroxyl or carboxyl,
   or
   is C₃-C₁₀-cycloalkyl optionally mono- or polysubstituted identically or differently by halogen, C₁-C₆-alkyl or C₁-C₆-alkoxy,
   or
   is phenyl optionally mono- or polysubstituted identically or differently by halogen, hydroxyl, cyano, carboxyl, C₁-C₆-alkyl, C₁-C₆-alkoxy, halo-C₁-C₆-alkyl, halo-C₁-C₆-alkoxy, C₃-C₁₀-cycloalkyl or a monocyclic heterocyclyl radical having 4 to 8 ring atoms, and
R³ is hydrogen or is C₁-C₆-alkyl, C₁-C₆-alkylcarbonyl, phenylsulphonyl or C₁-C₆-alkylsulphonyl, each optionally mono- or polysubstituted identically or differently by halogen, amino, hydroxyl, carboxyl, hydroxy-C₁-C₆-alkyl, C₁-C₆-alkoxy, C₁-C₆-alkoxy-C₁-C₆-alkyl, C₁-C₆-alkylamino or amino-C₁-C₆-alkyl, when X is a carbon atom,
   or
R³ is hydrogen or is C₁-C₆-alkyl, C₁-C₆-alkylcarbonyl, phenylsulphonyl or C₁-C₆-alkylsulphonyl, each optionally mono- or polysubstituted identically or differently by halogen, amino, hydroxyl, carboxyl, hydroxy-C₁-C₆-alkyl, C₁-C₆-alkoxy, C₁-C₆-alkoxy-C₁-C₆-alkyl, C₁-C₆-alkylamino or amino-C₁-C₆-alkyl, when X is a nitrogen atom,
Y is a —C(═O)OR¹², —C(═O)R¹³ or —C(═O)NR¹⁰R¹¹ group, or is phenyl, monocyclic heterocyclyl having 4 to 8 ring atoms or mono- or bicyclic heteroaryl having 5 to 10 ring atoms, in which phenyl, heteroaryl and heterocyclyl are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, phenoxy, halophenyl, phenyl-C₁-C₆-alkyl, phenyl-C₁-C₆-alkoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, or a monocyclic heterocyclyl radical which has 4 to 8 ring atoms and is itself optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl or halo-C₁-C₆-alkoxy,
R⁶ and R⁷ are each independently hydrogen, C₁-C₃-alkyl, cyclopropyl or di-C₁-C₃-alkylamino-C₁-C₃-alkyl,
R⁸ is hydroxyl, C₁-C₆-alkyl, C₁-C₆-alkoxy, halo-C₁-C₃-alkyl, hydroxy-C₁-C₃-alkyl, C₁-C₃-alkoxy-C₁-C₃-alkyl, C₃-C₈-cycloalkyl, phenyl, monocyclic heterocyclyl having 4 to 8 ring atoms or monocyclic heteroaryl having 5 or 6 ring atoms, where phenyl, heteroaryl and heterocyclyl are optionally mono- or disubstituted by halogen, C₁-C₃-alkoxy or C₁-C₃-alkyl,
R⁹ is C₁-C₆-alkyl,
R¹⁰ and R¹¹ are each independently hydrogen, C₁-C₆-alkyl or a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, C₁-C₆-alkylcarbonylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, phenoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ or a monocyclic heterocyclyl radical having 4 to 8 ring atoms,
   or
R¹⁰ and R¹¹ together with the adjacent nitrogen atom are a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, hydroxyl, amino, oxo, C₁-C₆-alkyl, C₁-C₆-alkoxy, C₁-C₆-alkylamino, C₁-C₆-alkylcarbonylamino, halo-C₁-C₆-alkyl, halo-C₁-C₆-alkoxy, C₃-C₁₀-cycloalkyl, phenyl, halophenyl, phenyl-C₁-C₆-alkyl, phenoxy, —C(═O)—NR⁶R⁷, —C(═O)—R⁸, —S(═O)₂—NR⁶R⁷, —S(═O)₂—R⁹, —NH—S(═O)₂—R⁹ or a monocyclic heterocyclyl radical having 4 to 8 ring atoms,
R₁₂ is C₁-C₆-alkyl, halo-C₁-C₃-alkyl, hydroxy-C₁-C₃-alkyl, C₁-C₃-alkoxy-C₁-C₃-alkyl, C₃-C₈-cycloalkyl, phenyl, a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, C₁-C₆-alkylcarbonylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, phenoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ or a monocyclic heterocyclyl radical having 4 to 8 ring atoms,
R¹³ is C₁-C₆-alkyl, halo-C₁-C₃-alkyl, hydroxy-C₁-C₃-alkyl, C₁-C₃-alkoxy-C₁-C₃-alkyl, C₃-C₈-cycloalkyl, phenyl, a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, each optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, C₁-C₆-alkylcarbonylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, phenoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ or a monocyclic heterocyclyl radical having 4 to 8 ring atoms,
   and their polymorphs, enantiomers, diastereomers, racemates, tautomers, solvates, physiologically acceptable salts and solvates of these salts.

Of very particular interest are those compounds of the general formula I in which
X is a carbon or nitrogen atom,
n and m are each independently 0 or 1,
p is 1,
R¹, R⁴ and R⁵ are each independently hydrogen or halogen,
R² is hydrogen or C₁-C₆-alkyl,
R³ is C₁-C₆-alkyl when X is a carbon atom,
   or
R³ is hydrogen or C₁-C₆-alkyl when X is a nitrogen atom,
Y is a —C(═O)OR¹² or —C(═O)NR¹⁰R¹¹ group, or is phenyl, monocyclic heterocyclyl having 4 to 8 ring atoms or monocyclic heteroaryl having 5 or 6 ring atoms, in which phenyl, heteroaryl and heterocyclyl are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, phenoxy, halophenyl, phenyl-C₁-C₆-alkyl, phenyl-C₁-C₆-alkoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, or a monocyclic heterocyclyl radical which has 4 to 8 ring atoms and is itself optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl or halo-C₁-C₆-alkoxy,
R⁶ and R⁷ are each independently hydrogen, C₁-C₃-alkyl, cyclopropyl or di-C₁-C₃-alkylamino-C₁-C₃-alkyl,
R⁸ is hydroxyl, C₁-C₆-alkyl, C₁-C₆-alkoxy, halo-C₁-C₃-alkyl, hydroxy-C₁-C₃-alkyl, C₁-C₃-alkoxy-C₁-C₃-alkyl, C₃-C₈-cycloalkyl, phenyl, monocyclic heterocyclyl having 4 to 8 ring atoms or monocyclic heteroaryl having 5 or 6 ring atoms, where phenyl, heteroaryl and heterocyclyl are optionally mono- or disubstituted by halogen, C₁-C₃-alkoxy or C₁-C₃-alkyl,
R⁹ is C₁-C₆-alkyl,
R¹⁰ and R¹¹ are each independently hydrogen, C₁-C₆-alkyl or a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, C₁-C₆-alkylcarbonylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, phenoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ or a monocyclic heterocyclyl radical having 4 to 8 ring atoms,
   or
R¹⁰ and R¹¹ together with the adjacent nitrogen atom are a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, hydroxyl, amino, oxo, C₁-C₆-alkyl, C₁-C₆-alkoxy, C₁-C₆-alkylamino, C₁-C₆-alkylcarbonylamino, halo-C₁-C₆-alkyl, halo-C₁-C₆-alkoxy, C₃-C₁₀-cycloalkyl, phenyl, halophenyl, phenyl-C₁-C₆-alkyl, phenoxy, —C(═O)—NR⁶R⁷, —C(═O)—R⁸, —S(═O)₂—NR⁶R⁷, —S(═O)₂—R⁹, —NH—S(═O)₂—R⁹ or a monocyclic heterocyclyl radical having 4 to 8 ring atoms,
R₁₂ is C₁-C₆-alkyl, halo-C₁-C₃-alkyl, hydroxy-C₁-C₃-alkyl, C₁-C₃-alkoxy-C₁-C₃-alkyl, C₃-C₈-cycloalkyl, phenyl, a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, C₁-C₆-alkylcarbonylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, phenoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ or a monocyclic heterocyclyl radical having 4 to 8 ring atoms,
   and their polymorphs, enantiomers, diastereomers, racemates, tautomers, solvates, physiologically acceptable salts and solvates of these salts.

Preference is given to those compounds of the general formula I in which
X is a carbon or nitrogen atom,
n and m are each independently 0 or 1,
p is 1,
R¹, R⁴ and R⁵ are each independently hydrogen or halogen,
R² is hydrogen or C₁-C₆-alkyl,
R³ is C₁-C₆-alkyl when X is a carbon atom,
   or
R³ is hydrogen or C₁-C₆-alkyl when X is a nitrogen atom,
Y is a —C(═O)OR¹² or —C(═O)NR¹⁰R¹¹ group, or is monocyclic heteroaryl which has 5 or 6 ring atoms and is optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, phenoxy, halophenyl, phenyl-C₁-C₆-alkyl, phenyl-C₁-C₆-alkoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, or a monocyclic heterocyclyl radical which has 4 to 8 ring atoms and is itself optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl or halo-C₁-C₆-alkoxy,
R⁶ and R⁷ are each independently hydrogen, C₁-C₃-alkyl, cyclopropyl or di-C₁-C₃-alkylamino-C₁-C₃-alkyl,
R⁸ is hydroxyl, C₁-C₆-alkyl, C₁-C₆-alkoxy, halo-C₁-C₃-alkyl, hydroxy-C₁-C₃-alkyl, C₁-C₃-alkoxy-C₁-C₃-alkyl, C₃-C₈-cycloalkyl, phenyl, monocyclic heterocyclyl having 4 to 8 ring atoms or monocyclic heteroaryl having 5 or 6 ring atoms, where phenyl, heteroaryl and heterocyclyl are optionally mono- or disubstituted by halogen, C₁-C₃-alkoxy or C₁-C₃-alkyl,
R⁹ is C₁-C₆-alkyl,
R¹⁰ and R¹¹ are each independently hydrogen, C₁-C₆-alkyl or a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, C₁-C₆-alkylcarbonylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, phenoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ or a monocyclic heterocyclyl radical having 4 to 8 ring atoms,
   or
R¹⁰ and R¹¹ together with the adjacent nitrogen atom are a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, hydroxyl, amino, oxo, C₁-C₆-alkyl, C₁-C₆-alkoxy, C₁-C₆-alkylamino, C₁-C₆-alkylcarbonylamino, halo-C₁-C₆-alkyl, halo-C₁-C₆-alkoxy, C₃-C₁₀-cycloalkyl, phenyl, halophenyl, phenyl-C₁-C₆-alkyl, phenoxy, —C(═O)—NR⁶R⁷, —C(═O)—R⁸, —S(═O)₂—NR⁶R⁷, —S(═O)₂—R⁹, —NH—S(═O)₂—R⁹ or a monocyclic heterocyclyl radical having 4 to 8 ring atoms,
R¹² is C₁-C₆-alkyl, C₃-C₈-cycloalkyl or a monocyclic or bicyclic heterocyclyl radical having 4 to 12 ring atoms, where the radicals mentioned are optionally mono- or polysubstituted identically or differently by halogen, oxo, C₁-C₆-alkyl or phenoxy,
   and their polymorphs, enantiomers, diastereomers, racemates, tautomers, solvates, physiologically acceptable salts and solvates of these salts.

Even more preference is given to those compounds of the general formula I in which
X is a carbon or nitrogen atom,
n and m are each independently 0 or 1,
p is 1,
R¹, R⁴ and R⁵ are each independently hydrogen or halogen,
R² is hydrogen or C₁-C₆-alkyl,
R³ is C₁-C₆-alkyl when X is a carbon atom,
   or
R³ is hydrogen or C₁-C₆-alkyl when X is a nitrogen atom,
Y is a —C(═O)OR¹² or —C(═O)NR¹⁰R¹¹ group, or is monocyclic heteroaryl which has 5 ring atoms and is optionally mono- or polysubstituted by C₁-C₆-alkyl or C₃-C₁₀-cycloalkyl,
R⁶ and R⁷ are each independently hydrogen, C₁-C₃-alkyl, cyclopropyl or di-C₁-C₃-alkylamino-C₁-C₃-alkyl,
R⁸ is hydroxyl, C₁-C₆-alkyl, C₁-C₆-alkoxy, halo-C₁-C₃-alkyl, hydroxy-C₁-C₃-alkyl, C₁-C₃-alkoxy-C₁-C₃-alkyl, C₃-C₈-cycloalkyl, phenyl, monocyclic heterocyclyl having 4 to 8 ring atoms or monocyclic heteroaryl having 5 or 6 ring atoms, where phenyl, heteroaryl and heterocyclyl are optionally mono- or disubstituted by halogen, C₁-C₃-alkoxy or C₁-C₃-alkyl,
R⁹ is C₁-C₆-alkyl,
R¹⁰ and R¹¹ are each independently hydrogen, C₁-C₆-alkyl or a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by oxo or —C(═O)—R⁸,
   or
R¹⁰ and R¹¹ together with the adjacent nitrogen atom are a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned may optionally include 1, 2 or 3 heteroatoms from the group of nitrogen, oxygen and sulphur, and are optionally mono- or polysubstituted identically or differently by halogen, cyano, amino, oxo, C₁-C₆-alkyl, C₁-C₆-alkoxy, C₁-C₆-alkylamino, C₁-C₆-alkylcarbonylamino, halo-C₁-C₆-alkyl, halo-C₁-C₆-alkoxy, C₃-C₁₀-cycloalkyl, phenyl, halophenyl, phenoxy, —C(═O)—NR⁶R⁷, C(═O)—R⁸, —S(═O)₂—NR⁶R⁷, —S(═O)₂—R⁹ or NH—S(═O)₂—R⁹,
R¹² is C₁-C₆-alkyl, C₃-C₈-cycloalkyl or a monocyclic or bicyclic heterocyclyl radical having 4 to 12 ring atoms, where the radicals mentioned are optionally mono- or polysubstituted identically or differently by halogen, oxo, C₁-C₆-alkyl or phenoxy, and their polymorphs, enantiomers, diastereomers, racemates, tautomers, solvates, physiologically acceptable salts and solvates of these salts.

Particular preference is given to those compounds of the general formula I in which
X is a carbon or nitrogen atom,
n and m are each independently 0 or 1,
p is 1,
R¹ is hydrogen or halogen,
R² is hydrogen or C₁-C₆-alkyl,
R³ is C₁-C₆-alkyl when X is a carbon atom,
   or
R³ is hydrogen or C₁-C₆-alkyl when X is a nitrogen atom,
R⁴ and R⁵ are hydrogen,
Y is a —C(═O)OR¹² or —C(═O)NR¹⁰R¹¹ group, or is monocyclic heteroaryl which has 5 ring atoms and is optionally mono- or polysubstituted by C₁-C₆-alkyl or C₃-C₁₀-cycloalkyl,
R⁸ is C₁-C₆-alkoxy,
R¹⁰ and R¹¹ are each independently hydrogen, C₁-C₆-alkyl or a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by oxo or —C(═O)—R⁸,
   or
R¹⁰ and R¹¹ together with the adjacent nitrogen atom are a monocyclic or bicyclic heterocyclyl radical which has 4 to 12 ring atoms and is optionally mono- or polysubstituted identically or differently by halogen, oxo, phenoxy or —C(═O)—R⁸,
R¹² is C₁-C₆-alkyl, C₃-C₅-cycloalkyl or a monocyclic or bicyclic heterocyclyl radical having 4 to 12 ring atoms, where the radicals mentioned are optionally mono- or polysubstituted identically or differently by halogen, oxo, C₁-C₆-alkyl or phenoxy,
   and their polymorphs, enantiomers, diastereomers, racemates, tautomers, solvates, physiologically acceptable salts and solvates of these salts.

Very particular preference is given to those compounds of the general formula I in which
X is a carbon or nitrogen atom,
n and m are each independently 0 or 1,
p is 1,
R¹ is hydrogen or chlorine,
R² is hydrogen or methyl,
R³ is methyl when X is a carbon atom,
   or
R³ is hydrogen or methyl when X is a nitrogen atom,
R⁴ and R⁵ are hydrogen,
Y is a —C(═O)OR¹² or —C(═O)NR¹⁰R¹¹ group or a monocyclic heteroaryl radical having 5 ring atoms of the structure:

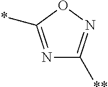

which may be substituted at “**” by methyl, ethyl, isopropyl, tert-butyl or cyclopropyl, and in which “*” denotes the attachment point to the rest of the molecule,
R¹⁰ is hydrogen or methyl,
R¹¹ is ethyl or one of the following groups:

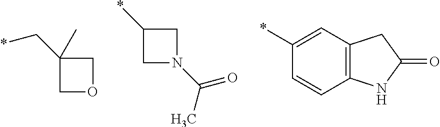

where “*” denotes the attachment point to the nitrogen atom in the —C(═O)NR¹⁰R¹¹ group defined in Y,
or
R¹⁰ and R¹¹ together with the adjacent nitrogen atom are one of the following groups:

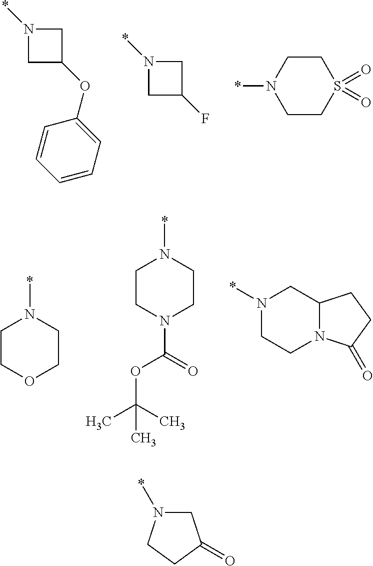

where “*” denotes the attachment point to the carbonyl group in the —C(═O)NR¹⁰R¹¹ group defined in Y,
R¹² is methyl or tert-butyl,
   and their polymorphs, enantiomers, diastereomers, racemates, tautomers, solvates, physiologically acceptable salts and solvates of these salts.

Preferably, R¹ is chlorine in the para position and R⁴ and R⁵ are hydrogen.

In the general formula (I), X is preferably a carbon or nitrogen atom.

In the general formula (I), X is preferably a carbon atom.

In the general formula (I), X is preferably a nitrogen atom.

In the general formula (I), n is preferably 0 or 1.

In the general formula (I), m is preferably 0 or 1.

In the general formula (I), p is preferably 1.

In the general formula (I), R¹, R⁴ and R⁵ may be the same or different and are preferably hydrogen or halogen.

In the general formula (I), R¹ is more preferably hydrogen or halogen.

In the general formula (I), R⁴ and R⁵ are more preferably hydrogen.

In the general formula (I), particular preference is given to those compounds in which R¹ is hydrogen or halogen and R⁴ and R⁵ are hydrogen.

In the general formula (I), R¹ is very especially preferably hydrogen or chlorine.

In the general formula (I), particular preference is given to those compounds in which R¹ is chlorine in the para position and R⁴ and R⁵ are hydrogen.

In the general formula (I), R² is preferably hydrogen or C₁-C₆-alkyl.

In the general formula (I), R² is very especially preferably hydrogen or methyl.

In the general formula (I), R² is very especially preferably hydrogen.

In the general formula (I), R² is very especially preferably methyl.

In the general formula (I), R³ is preferably C₁-C₆-alkyl when X is a carbon atom, or is hydrogen or C₁-C₆-alkyl when X is a nitrogen atom.

In the general formula (I), R³ is very especially preferably methyl when X is a carbon atom, or is hydrogen or methyl when X is a nitrogen atom.

In the general formula (I), Y is preferably a —C(═O)OR¹² or —C(═O)NR¹⁰R¹¹ group, or is monocyclic heteroaryl which has 5 or 6 ring atoms and is optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, phenoxy, halophenyl, phenyl-C₁-C₆-alkyl, phenyl-C₁-C₆-alkoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, or a monocyclic heterocyclyl radical which has 4 to 8 ring atoms and is itself optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl or halo-C₁-C₆-alkoxy.

In the general formula (I), Y is even more preferably a —C(═O)OR¹² or —C(═O)NR¹⁰R¹¹ group, or is monocyclic heteroaryl which has 5 ring atoms and is optionally mono- or polysubstituted by C₁-C₆-alkyl or C₃-C₁₀-cycloalkyl.

In the general formula (I), Y is especially preferably a —C(═O)OR¹² or —C(═O)NR¹⁰R¹¹ group, or is monocyclic heteroaryl which has 5 ring atoms and is optionally mono- or polysubstituted by C₁-C₆-alkyl or C₃-C₁₀-cycloalkyl.

In the general formula (I), Y is very especially preferably a —C(═O)OR¹² or —C(═O)NR¹⁰R¹¹ group, or a monocyclic heteroaryl radical having 5 ring atoms of the structure:

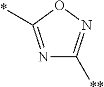

which may be substituted at “**” by methyl, ethyl, isopropyl, tert-butyl or cyclopropyl, and in which “*” denotes the attachment point to the rest of the molecule.

In the general formula (I), preference is given to those compounds in which R⁶ and R⁷ are each independently hydrogen, C₁-C₃-alkyl, cyclopropyl or di-C₁-C₃-alkylamino-C₁-C₃-alkyl.

In the general formula (I), R⁸ is preferably hydroxyl, C₁-C₆-alkyl, C₁-C₆-alkoxy, halo-C₁-C₃-alkyl, hydroxy-C₁-C₃-alkyl, C₁-C₃-alkoxy-C₁-C₃-alkyl, C₃-C₈-cycloalkyl, phenyl, monocyclic heterocyclyl having 4 to 8 ring atoms or monocyclic heteroaryl having 5 or 6 ring atoms, in which phenyl, heteroaryl and heterocyclyl are optionally mono- or disubstituted by halogen, C₁-C₃-alkoxy or C₁-C₃-alkyl.

In the general formula (I), R⁸ is preferably hydroxyl, C₁-C₆-alkyl, C₁-C₆-alkoxy, C₃-C₈-cycloalkyl, phenyl, monocyclic heterocyclyl having 4 to 8 ring atoms or monocyclic heteroaryl having 5 or 6 ring atoms.

In the general formula (I), R⁸ is especially preferably C₁-C₆-alkoxy.

In the general formula (I), R⁹ is preferably C₁-C₆-alkyl.

In the general formula (I), preference is given to those compounds in which R¹⁰ and R¹¹ are each independently hydrogen, C₁-C₆-alkyl or a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, C₁-C₆-alkylcarbonylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, phenoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ or a monocyclic heterocyclyl radical having 4 to 8 ring atoms,

or
in which R¹⁰ and R¹¹ together with the adjacent nitrogen atom are a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, hydroxyl, amino, oxo, C₁-C₆-alkyl, C₁-C₆-alkoxy, C₁-C₆-alkylamino, C₁-C₆-alkylcarbonylamino, halo-C₁-C₆-alkyl, halo-C₁-C₆-alkoxy, C₃-C₁₀-cycloalkyl, phenyl, halophenyl, phenyl-C₁-C₆-alkyl, phenoxy, —C(═O)—NR⁶R⁷, —C(═O)—R⁸, —S(═O)₂—NR⁶R⁷, —S(═O)₂—R⁹, —NH—S(═O)₂—R⁹ or a monocyclic heterocyclyl radical having 4 to 8 ring atoms.

In the general formula (I), preference is given to those compounds in which R¹⁰ and R¹¹ are each independently hydrogen, C₁-C₆-alkyl or a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, C₁-C₆-alkylcarbonylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, phenoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ or a monocyclic heterocyclyl radical having 4 to 8 ring atoms.

In the general formula (I), preference is given to those compounds in which R¹⁰ and R¹¹ together with the adjacent nitrogen atom are a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, hydroxyl, amino, oxo, C₁-C₆-alkyl, C₁-C₆-alkoxy, C₁-C₆-alkylamino, C₁-C₆-alkylcarbonylamino, halo-C₁-C₆-alkyl, halo-C₁-C₆-alkoxy, C₃-C₁₀-cycloalkyl, phenyl, halophenyl, phenyl-C₁-C₆-alkyl, phenoxy, —C(═O)—NR⁶R⁷, —C(═O)—R⁸, —S(═O)₂—NR⁶R⁷, —S(═O)₂—R⁹, —NH—S(═O)₂—R⁹ or a monocyclic heterocyclyl radical having 4 to 8 ring atoms.

In the general formula (I), even more preference is given to those compounds in which R¹⁰ and R¹¹ are each independently hydrogen, C₁-C₆-alkyl or a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by oxo or —C(═O)—R⁸,

or
in which R¹⁰ and R¹¹ together with the adjacent nitrogen atom are a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned may optionally include 1, 2 or 3 heteroatoms from the group of nitrogen, oxygen and sulphur, and are optionally mono- or polysubstituted identically or differently by halogen, cyano, amino, oxo, C₁-C₆-alkyl, C₁-C₆-alkoxy, C₁-C₆-alkylamino, C₁-C₆-alkylcarbonylamino, halo-C₁-C₆-alkyl, halo-C₁-C₆-alkoxy, C₃-C₁₀-cycloalkyl, phenyl, halophenyl, phenoxy, —C(═O)—NR⁶R⁷, —C(═O)—R⁸, —S(═O)₂—NR⁶R⁷, —S(═O)₂—R⁹ or NH—S(═O)₂—R⁹.

In the general formula (I), even more preference is given to those compounds in which R¹⁰ and R¹¹ are each independently hydrogen, C₁-C₆-alkyl or a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by oxo or —C(═O)—R⁸.

In the general formula (I), even more preference is given to those compounds in which R¹⁰ and R¹¹ together with the adjacent nitrogen atom are a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned may optionally include 1, 2 or 3 heteroatoms from the group of nitrogen, oxygen and sulphur, and are optionally mono- or polysubstituted identically or differently by halogen, cyano, amino, oxo, C₁-C₆-alkyl, C₁-C₆-alkoxy, C₁-C₆-alkylamino, C₁-C₆-alkylcarbonylamino, halo-C₁-C₆-alkyl, halo-C₁-C₆-alkoxy, C₃-C₁₀-cycloalkyl, phenyl, halophenyl, phenoxy, —C(═O)—NR⁶R⁷, —C(═O)—R⁸, —S(═O)₂—NR⁶R⁷, —S(═O)₂—R⁹ or NH—S(═O)₂—R⁹.

In the general formula (I), particular preference is given to those compounds in which R¹⁰ and R¹¹ are each independently hydrogen, C₁-C₆-alkyl or a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by oxo or —C(═O)—R⁸,

or
in which R¹⁰ and R¹¹ together with the adjacent nitrogen atom are a monocyclic or bicyclic heterocyclyl radical which has 4 to 12 ring atoms and is optionally mono- or polysubstituted identically or differently by halogen, oxo, phenoxy or —C(═O)—R⁸.

In the general formula (I), particular preference is given to those compounds in which R¹⁰ and R¹¹ are each independently hydrogen, C₁-C₆-alkyl or a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by oxo or —C(═O)—R⁸.

In the general formula (I), particular preference is given to those compounds in which R¹⁰ and R¹¹ together with the adjacent nitrogen atom are a monocyclic or bicyclic heterocyclyl radical which has 4 to 12 ring atoms and is optionally mono- or polysubstituted identically or differently by halogen, oxo, phenoxy or —C(═O)—R⁸.

In the general formula (I), particular preference is further given to those compounds in which R¹⁰ is hydrogen or C₁-C₃-alkyl and R¹¹ is independently hydrogen, C₁-C₆-alkyl or a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by oxo or —C(═O)—R⁸.

In the general formula (I), particular preference is given to those compounds in which R¹⁰ is hydrogen or methyl and R¹¹ is ethyl or one of the following groups:

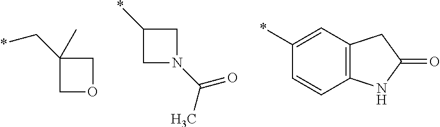

where “*” denotes the attachment point to the nitrogen atom in the —C(═O)NR¹⁰R¹¹ group defined in Y,
or R¹⁰ and R¹¹ together with the adjacent nitrogen atom are one of the following groups:

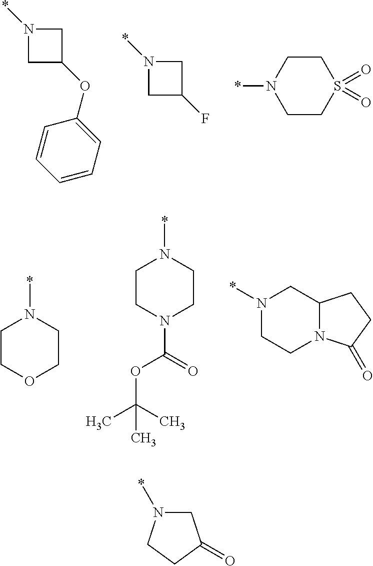

where “*” denotes the attachment point to the carbonyl group in the —C(═O)NR¹⁰R¹¹ group defined in

Y.

In the general formula (I), particular preference is given to those compounds in which R¹⁰ is hydrogen or methyl and R¹¹ is ethyl or one of the following groups:

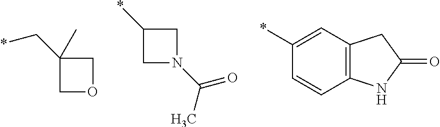

where “*” denotes the attachment point to the nitrogen atom in the —C(═O)NR¹⁰R¹¹ group defined in Y.

In the general formula (I), particular preference is given to those compounds in which R¹⁰ and R¹¹ together with the adjacent nitrogen atom are one of the following groups:

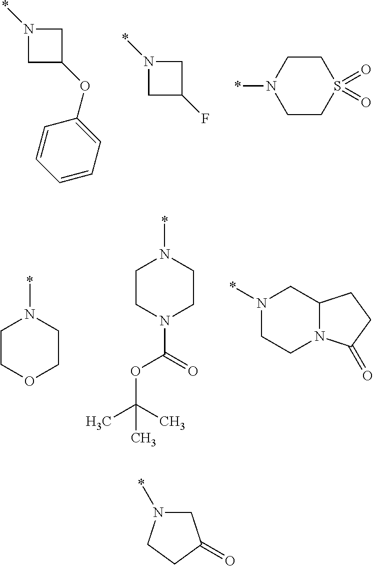

where “*” denotes the attachment point to the carbonyl group in the —C(═O)NR¹⁰R¹¹ group defined in Y.

In the general formula (I), R¹² is preferably C₁-C₆-alkyl, C₃-C₈-cycloalkyl or a monocyclic or bicyclic heterocyclyl radical having 4 to 12 ring atoms, where the radicals mentioned are optionally mono- or polysubstituted identically or differently by halogen, oxo, C₁-C₆-alkyl or phenoxy.

In the general formula (I), R¹² is especially preferably C₁-C₆-alkyl or C₃-C₈-cycloalkyl.

In the general formula (I), R¹² is very especially preferably methyl or tert-butyl.

When X in the general formula I is nitrogen, tautomeric forms of the inventive compounds may be possible. In this case, the circle is supposed to represent both possible positions of the double bonds, as shown here:

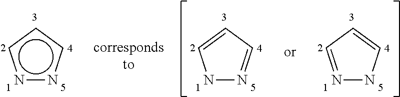

The invention is based on the following definitions:

### Alkyl

Alkyl is a linear or branched saturated monovalent hydrocarbon radical having generally 1 to 6 (C₁-C₆-alkyl), preferably 1 to 4 (C₁-C₄-alkyl) and especially preferably 1 to 3 (C₁-C₃-alkyl) carbon atoms.

Preferred examples include:

methyl, ethyl, propyl, butyl, pentyl, hexyl, isopropyl, isobutyl, sec-butyl, tert-butyl, isopentyl, 2-methylbutyl, 1-methylbutyl, 1-ethylpropyl, 1,2-dimethylpropyl, neopentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl, 1,2-dimethylbutyl.

Particular preference is given to a methyl, ethyl, propyl, isopropyl or tert-butyl radical.

### Cycloalkyl

Cycloalkyl is a monocyclic saturated monovalent hydrocarbon radical having generally 3 to 10 (C₃-C₁₀-cycloalkyl), preferably 3 to 8 (C₃-C₈-cycloalkyl) and especially preferably 3 to 7 (C₃-C₇-cycloalkyl) carbon atoms.

Preferred examples of monocyclic cycloalkyl radicals include:

cyclopropyl, cyclobutyl, cyclopentyl, cyclohexyl and cycloheptyl.

Particular preference is given to a cyclopropyl, cyclopentyl or cyclohexyl radical.

### Alkoxy

Alkoxy is a linear or branched saturated alkyl ether radical of the formula —O-alkyl having generally 1 to 6 (C₁-C₆-alkoxy), preferably 1 to 4 (C₁-C₄-alkoxy) and especially preferably 1 to 3 (C₁-C₃-alkoxy) carbon atoms.

Preferred examples include:

methoxy, ethoxy, n-propoxy, isopropoxy, tert-butoxy, n-pentyloxy and n-hexyloxy.

### Alkoxyalkyl

Alkoxyalkyl is an alkoxy-substituted alkyl radical, for example C₁-C₆-alkoxy-C₁-C₆-alkyl or C₁-C₃-alkoxy-C₁-C₃-alkyl.

C₁-C₆-Alkoxy-C₁-C₆-alkyl means that the alkoxyalkyl group is bonded to the rest of the molecule via the alkyl moiety.

### Oxo

Oxo, an oxo group or an oxo substituent, is understood to mean a double-bonded oxygen atom ═O. Oxo may be bonded to atoms of suitable valency, for example to a saturated carbon atom or to sulphur.

Preference is given to bonding to carbon to form a carbonyl group or bonding to sulphur to form a sulphinyl or sulphonyl group.

### Alkylamino

Alkylamino is an amino radical having one or two (independently selected) alkyl substituents having generally 1 to 6 (C₁-C₆-alkylamino) and preferably 1 to 3 (C₁-C₃-alkylamino) carbon atoms. (C₁-C₃)-Alkylamino is, for example, a monoalkylamino radical having 1 to 3 carbon atoms or a dialkylamino radical having 1 to 3 carbon atoms each per alkyl substituent.

Preferred examples include:

methylamino, ethylamino, n-propylamino, isopropylamino, tert-butylamino, n-pentylamino, n-hexylamino, N,N-dimethylamino, N,N-diethylamino, N-ethyl-N-methylamino, N-methyl-N-n-propylamino, N-isopropyl-N-n-propylamino, N-tert-butyl-N-methylamino, N-ethyl-N-n-pentylamino and N-n-hexyl-N-methylamino.

### Alkylaminocarbonyl

Alkylaminocarbonyl is the alkylamino-C(═O)— group having one or two (independently selected) alkyl substituents having generally 1 to 6 (C₁-C₆-alkylaminocarbonyl) and preferably 1 to 3 (C₁-C₃-alkylaminocarbonyl) carbon atoms.

### Alkylcarbonyl

Alkylcarbonyl is the —C(═O)-alkyl group having generally 1 to 6 (C₁-C₆-alkylcarbonyl), preferably 1 to 4 and especially preferably 1 to 3 carbon atoms in the alkyl moiety.

Examples include acetyl and propanoyl.

### Alkylcarbonylamino

Alkylcarbonylamino is the alkyl-C(═O)—NH— group having generally 1 to 6 (C₁-C₆-alkylcarbonylamino), preferably 1 to 4 and especially preferably 1 to 3 carbon atoms in the alkyl moiety.

### Alkylsulphonyl

Alkylsulphonyl is a linear or branched saturated radical of the formula —S(═O)₂-alkyl having generally 1 to 6 (C₁-C₆-alkylsulphonyl), preferably 1 to 4 (C₁-C₄-alkylsulphonyl) and especially preferably 1 to 3 (C₁-C₃-alkylsulphonyl) carbon atoms.

Preferred examples include:

methylsulphonyl, ethylsulphonyl, propylsulphonyl.

### Alkylaminosulphonyl

Alkylaminosulphonyl is the alkylamino-S(═O)₂— group having one or two (independently selected) alkyl substituents having generally 1 to 6 (C₁-C₆-alkylaminosulphonyl) and preferably 1 to 3 carbon atoms.

Preferred examples include:

methylaminosulphonyl, ethylaminosulphonyl, dimethylaminosulphonyl.

### Phenylalkyl

Phenyl-C₁-C₆-alkyl is understood to mean a group composed of an optionally substituted phenyl radical and a C₁-C₆-alkyl group, and bonded to the rest of the molecule via the C₁-C₆-alkyl group. The alkyl radical here is as defined above under alkyl.

Examples include benzyl, phenethyl, phenylpropyl, phenylpentyl, preference being given to benzyl.

### Phenylalkoxy

Phenyl-C₁-C₆-alkoxy is understood to mean a group composed of an optionally substituted phenyl radical and a C₁-C₆-alkoxy group, and bonded to the rest of the molecule via the C₁-C₆-alkoxy group. The alkoxy radical here is as defined above under alkoxy.

Examples include benzoxy, phenethoxy, phenylpropyloxy, phenylpentyloxy, preference being given to benzoxy.

### Phenylsulphonyl

Phenylsulphonyl is understood to mean a group composed of an optionally substituted phenyl radical and an —S(═O)₂ group.

Examples include phenylsulphonyl, o- or p-toluylsulphonyl, m-chlorophenylsulphonyl.

### Heteroatoms

Heteroatoms are understood to mean oxygen, nitrogen or sulphur atoms.

### Heteroaryl

Heteroaryl means a monovalent aromatic ring system having 1, 2, 3, 4, 5 or 6 heteroatoms. The heteroatoms may be nitrogen atoms, oxygen atoms and/or sulphur atoms. The bonding valency may be at any aromatic carbon atom or at a nitrogen atom.

A monocyclic heteroaryl radical according to the present invention has 5 or 6 ring atoms.

Heteroaryl radicals having 5 ring atoms include, for example, the following rings: thienyl, thiazolyl, furyl, pyrrolyl, oxazolyl, imidazolyl, pyrazolyl, isoxazolyl, isothiazolyl, oxadiazolyl, triazolyl, tetrazolyl and thiadiazolyl.

Heteroaryl radicals having 6 ring atoms include, for example, the following rings: pyridyl, pyridazinyl, pyrimidinyl, pyrazinyl and triazinyl.

A bicyclic heteroaryl radical in accordance with the present invention has 9 or 10 ring atoms.

Heteroaryl radicals having 9 ring atoms include, for example, the following rings: phthalidyl, thiophthalidyl, indolyl, isoindolyl, indazolyl, benzothiazolyl, benzofuryl, benzothienyl, benzimidazolyl, benzoxazolyl, azocinyl, indolizinyl, purinyl, indolinyl.

Heteroaryl radicals having 10 ring atoms include, for example, the rings: isoquinolinyl, quinolinyl, quinolizinyl, quinazolinyl, quinoxalinyl, cinnolinyl, phthalazinyl, 1,7- and 1,8-naphthyridinyl, pteridinyl, chromanyl.

### Partly Saturated Bicyclic Aryl and Partly Saturated Bicyclic Heteroaryl

A partly saturated bicyclic aryl radical or heteroaryl radical is a bicyclic group consisting of a phenyl radical or a monocyclic 5- or 6-membered heteroaryl radical fused via each of two directly adjacent ring atoms to an aliphatic cyclic radical which has 4 to 7 ring atoms and may optionally contain one or two heteroatoms which may be the same or different. The heteroatoms may be nitrogen atoms, oxygen atoms and/or sulphur atoms.

Partly saturated bicyclic aryl radicals include, for example, the following groups: tetrahydronaphthyl, 2,3-dihydro-1,4-benzodioxinyl, 2,3-dihydro-1-benzofuranyl and 1,3-benzodioxolyl.

Partly saturated bicyclic heteroaryl radicals include, for example, the following groups: 5,6,7,8-tetrahydroquinoline and 5,6,7,8-tetrahydroisoquinoline.

### Monocyclic Heterocyclyl

Monocyclic heterocyclyl means a nonaromatic monocyclic ring system having 1, 2, 3, 4, 5 or 6 heteroatoms. The heteroatoms may be nitrogen atoms, oxygen atoms and/or sulphur atoms. A monocyclic heterocyclyl ring according to the present invention may have 4 to 8, preferably 4 to 7, especially preferably 5 or 6 ring atoms.

Preferred examples of monocyclic heterocyclyl radicals having 4 ring atoms are as follows: azetidinyl, oxetanyl.

Preferred examples of monocyclic heterocyclyl radicals having 5 ring atoms are as follows: pyrrolidinyl, imidazolidinyl, pyrazolidinyl, pyrrolinyl, dioxolanyl and tetrahydrofuranyl.

Preferred examples of monocyclic heterocyclyl radicals having 6 ring atoms are as follows: piperidinyl, piperazinyl, morpholinyl, dioxanyl, tetrahydropyranyl and thiomorpholinyl.

Preferred examples of monocyclic heterocyclyl radicals having 7 ring atoms are as follows: azepanyl, oxepanyl, 1,3-diazepanyl, 1,4-diazepanyl.

Preferred examples of monocyclic heterocyclyl radicals having 8 ring atoms are as follows: oxocanyl, azocanyl.

Among the monocyclic heterocyclyl radicals are 3- to 8-membered, preferably 4- to 7-membered and especially preferably 5- to 6-membered saturated heterocyclyl radicals having up to two heteroatoms from the group of O, N and S.

Very particular preference is given to morpholinyl, piperidinyl and pyrrolidinyl.

### Bicycloalkyl and Heterobicycloalkyl

C₆-C₁₂-Bicycloalkyl or C₆-C₁₂-heterobicycloalkyl with one, two, three or four carbon atoms replaced by heteroatoms as defined above in any combination is understood to mean a fusion of two saturated ring systems which share two directly adjacent atoms. Examples are bicyclo[2.2.0]hexyl, bicyclo[3.3.0]octyl, bicyclo[4.4.0]decyl, bicyclo[5.4.0]undecyl, bicyclo[3.2.0]heptyl, bicyclo[4.2.0]octyl, bicyclo[5.2.0]nonyl, bicyclo[6.2.0]decyl, bicyclo[4.3.0]nonyl, bicyclo[5.3.0]decyl, bicyclo[6.3.0]undecyl and bicyclo[5.4.0]undecyl, including the variants modified by heteroatoms, for example azabicyclo[3.3.0]octyl, azabicyclo[4.3.0]nonyl, diazabicyclo[4.3.0]nonyl, oxazabicyclo[4.3.0]nonyl, thiazabicyclo[4.3.0]nonyl or azabicyclo[4.4.0]decyl, and the further possible combinations as per the definition. Preference is given to C₆-C₁₀-heterobicycloalkyl. The term “monocyclic or bicyclic heterocyclyl radical having 4 to 12 ring atoms” encompasses the radicals defined above as “monocyclic heterocyclyl” and “C₆-C₁₂-heterobicycloalkyl”.

### Halogen

The term “halogen” includes fluorine, chlorine, bromine and iodine.

Preference is given to fluorine, chlorine and bromine, in particular fluorine or chlorine.

### Haloalkyl:

Haloalkyl is an alkyl radical having at least one halogen substituent.

A halo-C₁-C₆-alkyl radical is an alkyl radical having 1-6 carbon atoms and at least one halogen substituent. If two or more halogen substituents are present, these may also be different from one another.

Preferred examples include:

the trifluoromethyl, 2,2,2-trifluoroethyl, pentafluoroethyl, 4,4,5,5,5-pentafluoropentyl or 3,3,4,4,5,5,5-heptafluoropentyl group.

Preference is given to perfluorinated alkyl radicals such as trifluoromethyl or pentafluoroethyl.

### Haloalkoxy

Haloalkoxy is an alkoxy radical having at least one halogen substituent.

A halo-C₁-C₆-alkoxy radical is an alkoxy radical having 1-6 carbon atoms and at least one halogen substituent. If two or more halogen substituents are present, these may also be different from one another. Preference is given to fluoroalkoxy radicals.

Preferred examples include:

the trifluoromethoxy or 2,2,2-trifluoroethoxy radical.

### Hydroxyalkyl

Hydroxyalkyl is an alkyl radical having at least one hydroxyl substituent.

A hydroxy-C₁-C₆-alkyl radical is an alkyl radical having 1-6 carbon atoms and at least one hydroxyl substituent.

### Aminoalkyl

Aminoalkyl is an alkyl radical having at least one amino substituent.

An amino-C₁-C₆-alkyl radical is an alkyl radical consisting of 1-6 carbon atoms and at least one amino substituent.

### Alkylaminoalkyl

Alkylaminoalkyl is an alkyl radical substituted by alkylamino as defined above, for example C₁-C₆-alkylamino-C₁-C₆-alkyl or C₁-C₃-alkylamino-C₁-C₃-alkyl.

C₁-C₆-Alkylamino-C₁-C₆-alkyl means here that the alkylaminoalkyl group is bonded to the rest of the molecule via the alkyl moiety.

Dialkylaminoalkyl, for example di-C₁-C₃-alkylamino-C₁-C₃-alkyl, means that the aforementioned alkylamino moiety necessarily contains two alkyl groups which may be the same or different. Examples of alkylaminoalkyl are N,N-dimethylaminoethyl, N,N-dimethylaminomethyl, N,N-diethylaminoethyl, N,N-dimethylaminopropyl, N-methylaminoethyl, N-methylaminomethyl.

Especial preference is given to the following compounds of the general formula I:
2-(4S)-[6-(4-chlorophenyl)-1,7,8-trimethyl-4,8-dihydropyrrolo[3,4-f][1,2,4]triazolo[4,3-a][1,4]diazepin-4-yl]acetic acid methyl ester;
2-(4S)-(1,7,8-trimethyl-6-phenyl-4,8-dihydropyrrolo[3,4-f][1,2,4]triazolo[4,3-a][1,4]diazepin-4-yl)acetic acid methyl ester;
(−)-2-(4S)-[6-(4-chlorophenyl)-1,7,8-trimethyl-4,8-dihydropyrrolo[3,4-f][1,2,4]triazolo[4,3-a][1,4]diazepin-4-yl]acetic acid tert-butyl ester;
(−)-2-(4S)-(1,7,8-trimethyl-6-phenyl-4,8-dihydropyrrolo[3,4-f][1,2,4]triazolo[4,3-a][1,4]diazepin-4-yl)acetic acid tert-butyl ester;
(−)-2-[(4S)-6-(4-chlorophenyl)-1,7,8-trimethyl-4,8-dihydropyrrolo[3,4-f][1,2,4]triazolo[4,3-a][1,4]diazepin-4-yl]-1-(3-fluoroazetidin-1-yl)ethan-1-one;
2-[(4S)-6-(4-chlorophenyl)-1,8-dimethyl-4,8-dihydropyrazolo[3,4-f][1,2,4]triazolo[4,3-a][1,4]diazepin-4-yl]acetic acid methyl ester;
2-[(4S)-6-(4-chlorophenyl)-1,7-dimethyl-4,7-dihydropyrazolo[3,4-f][1,2,4]triazolo[4,3-a][1,4]diazepin-4-yl]acetic acid methyl ester;
(−)-2-[(4S)-6-(4-chlorophenyl)-1,7,8-trimethyl-4,8-dihydropyrrolo[3,4-f][1,2,4]triazolo[4,3-a][1,4]diazepin-4-yl]-1-(1,1-dioxidothiomorpholino)ethan-1-one;
N-(1-acetylazetidin-3-yl)-2-(4S)-[6-(4-chlorophenyl)-1,7,8-trimethyl-4,8-dihydropyrrolo[3,4-f][1,2,4]triazolo[4,3-a][1,4]diazepin-4-yl]acetamide;
(−)-2-[(4S)-6-(4-chlorophenyl)-1,7,8-trimethyl-4,8-dihydropyrrolo[3,4-f][1,2,4]triazolo[4,3-a][1,4]diazepin-4-yl]-N-ethylacetamide;
(−)-2-[(4S)-6-(4-chlorophenyl)-1,7,8-trimethyl-4,8-dihydropyrrolo[3,4-f][1,2,4]triazolo[4,3-a][1,4]diazepin-4-yl]-N-methyl-N-[(3-methyloxetan-3-yl)methyl]acetamide;
(−)-1-{[(4S)-6-(4-chlorophenyl)-1,7,8-trimethyl-4,8-dihydropyrrolo[3,4-f][1,2,4]triazolo[4,3-a][1,4]diazepin-4-yl]acetyl}pyrrolidin-3-one;
2-[(4S)-6-(4-chlorophenyl)-1,7,8-trimethyl-4,8-dihydropyrrolo[3,4-f][1,2,4]triazolo[4,3-a][1,4]diazepin-4-yl]-N-(2-oxo-2,3-dihydro-1H-indol-5-yl)acetamide;
2-[6-(4-chlorophenyl)-1,8-dimethyl-4,8-dihydropyrazolo[3,4-f][1,2,4]triazolo[4,3-a][1,4]diazepin-4-yl]-1-(1,1-dioxidothiomorpholin-4-yl)ethanone;
2-[(4S)-6-(4-chlorophenyl)-1,8-dimethyl-4,8-dihydropyrazolo[3,4-f][1,2,4]triazolo[4,3-a][1,4]diazepin-4-yl]-1-(1,1-dioxidothiomorpholin-4-yl) ethanone;
2-[6-(4-chlorophenyl)-1,8-dimethyl-4,8-dihydropyrazolo[3,4-f][1,2,4]triazolo[4,3-a][1,4]diazepin-4-yl]-1-(morpholin-4-yl)ethanone;
2-[6-(4-chlorophenyl)-1,8-dimethyl-4,8-dihydropyrazolo[3,4-f][1,2,4]triazolo[4,3-a][1,4]diazepin-4-yl]-1-(3-fluoroazetidin-1-yl) ethanone;
2-[6-(4-chlorophenyl)-1,8-dimethyl-4,8-dihydropyrazolo[3,4-f][1,2,4]triazolo[4,3-a][1,4]diazepin-4-yl]-N-ethylacetamide;
1-(morpholin-4-yl)-2-[(4S)-1,7,8-trimethyl-6-phenyl-4,8-dihydropyrrolo[3,4-f][1,2,4]triazolo[4,3-a][1,4]diazepin-4-yl]ethanone;
(−)-2-[(4S)-6-(4-chlorophenyl)-1,7,8-trimethyl-4,8-dihydropyrrolo[3,4-f][1,2,4]triazolo[4,3-a][1,4]diazepin-4-yl]-1-(3-phenoxyazetidin-1-yl)ethanone;
(−)-2-[(4S)-6-(4-chlorophenyl)-1,7,8-trimethyl-4,8-dihydropyrrolo[3,4-f][1,2,4]triazolo[4,3-a][1,4]diazepin-4-yl]-1-(morpholin-4-yl)ethanone;
(−)-4-{[(4S)-6-(4-chlorophenyl)-1,7,8-trimethyl-4,8-dihydropyrrolo[3,4-f][1,2,4]triazolo[4,3-a][1,4]diazepin-4-yl]acetyl}piperazine-1-carboxylic acid tert-butyl ester;
(−)-2-{[(4S)-6-(4-chlorophenyl)-1,7,8-trimethyl-4,8-dihydropyrrolo[3,4-f][1,2,4]triazolo[4,3-a][1,4]diazepin-4-yl]acetyl}hexahydropyrrolo[1,2-a]pyrazin-6(2H)-one;
6-(4-chlorophenyl)-4-[(3-cyclopropyl-1,2,4-oxadiazol-5-yl)methyl]-1,7,8-trimethyl-4,8-dihydropyrrolo[3,4-f][1,2,4]triazolo[4,3-a][1,4]diazepine;
6-(4-chlorophenyl)-1,7,8-trimethyl-4-[(3-methyl-1,2,4-oxadiazol-5-yl)methyl]-4,8-dihydropyrrolo[3,4-f][1,2,4]triazolo[4,3-a][1,4]diazepine;
(4S)-6-(4-chlorophenyl)-1,7,8-trimethyl-4-[(3-methyl-1,2,4-oxadiazol-5-yl)methyl]-4,8-dihydropyrrolo[3,4-f][1,2,4]triazolo[4,3-a][1,4]diazepine;
6-(4-chlorophenyl)-1,7,8-trimethyl-4-[(3-ethyl-1,2,4-oxadiazol-5-yl)methyl]-4,8-dihydropyrrolo[3,4-f][1,2,4]triazolo[4,3-a][1,4]diazepine;
(4S)-6-(4-chlorophenyl)-1,7,8-trimethyl-4-[(3-ethyl-1,2,4-oxadiazol-5-yl)methyl]-4,8-dihydropyrrolo[3,4-f][1,2,4]triazolo[4,3-a][1,4]diazepine;
6-(4-chlorophenyl)-1,7,8-trimethyl-4-{[3-(propan-2-yl)-1,2,4-oxadiazol-5-yl]methyl}-4,8-dihydropyrrolo[3,4-f][1,2,4]triazolo[4,3-a][1,4]diazepine;
(4S)-6-(4-chlorophenyl)-1,7,8-trimethyl-4-{[3-(propan-2-yl)-1,2,4-oxadiazol-5-yl]methyl}-4,8-dihydropyrrolo[3,4-f][1,2,4]triazolo[4,3-a][1,4]diazepine and
4-[(3-tert-butyl-1,2,4-oxadiazol-5-yl)methyl]-6-(4-chlorophenyl)-1,7,8-trimethyl-4,8-dihydropyrrolo[3,4-f][1,2,4]triazolo[4,3-a][1,4]diazepine.

The specific radical definitions given in the particular combinations or preferred combinations of radicals are, irrespective of the particular combinations of radicals specified, also replaced as desired by radical definitions of other combination.

Very particular preference is given to combinations of two or more of the abovementioned preferred ranges.

Inventive compounds are the compounds of the formula (I) and their salts, solvates and solvates of the salts, the compounds, encompassed by formula (I), of the formulae mentioned below and their salts, solvates and solvates of the salts and the compounds encompassed by the formula (I), mentioned below as embodiments and their salts, solvates and solvates of the salts if the compounds, encompassed by the formula (I), mentioned below are not already salts, solvates and solvates of the salts.

The present invention is likewise considered to encompass the use of the salts of the inventive compounds.

Preferred salts in the context of the present invention are physiologically acceptable salts of the inventive compounds. The invention also encompasses salts which themselves are unsuitable for pharmaceutical applications but which can be used, for example, for the isolation or purification of the inventive compounds.

Physiologically acceptable salts of the inventive compounds include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

Physiologically acceptable salts of the inventive compounds furthermore include base addition salts, for example of alkali metals such as sodium or potassium, of alkaline earth metals such as calcium or magnesium, or of ammonium salts derived from ammonia or organic amines having 1 to 16 carbon atoms, for example methylamine, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine, N-methylpiperidine, N-methylglucamine, dimethylglucamine, ethylglucamine, 1,6-hexadiamine, glucosamine, sarcosine, serinol, tris(hydroxymethyl)aminomethane, aminopropanediol, Sovak base and/or 1-amino-2,3,4-butanetriol. Furthermore, the inventive compounds may form base addition salts with quaterary ammonium ions which can be obtained, for example, by quaternization of corresponding amines with agents such as lower alkyl halides, for example methyl, ethyl, propyl and butyl chlorides, methyl, ethyl, propyl and butyl bromides, and methyl, ethyl, propyl and butyl iodides, dialkyl sulphates such as dimethyl, diethyl, dibutyl and diamyl sulphate, long-chain halides such as decyl, lauryl, myristyl and stearyl chlorides, decyl, lauryl, myristyl and stearyl bromides, and decyl, lauryl, myristyl and stearyl iodides, or arylalkyl halides such as benzyl bromide or phenethyl bromide. Examples of such quaternary ammonium ions are tetramethylammonium, tetraethylammonium, tetra(n-propyl)ammonium, tetra(n-butyl)ammonium and also benzyltrimethylammonium.

The present invention further provides all the possible crystalline and polymorphous forms of the inventive compounds, where the polymorphs may be present either as single polymorphs or as a mixture of a plurality of polymorphs in all concentration ranges.

The present invention further provides medicaments comprising the inventive compounds and at least one or more further active ingredients, especially for prophylaxis and/or treatment of neoplastic disorders.

Solvates in the context of the invention are described as those forms of the inventive compounds which form a complex in the solid or liquid state by coordination with solvent molecules. Hydrates are a specific form of the solvates in which the coordination is with water. Solvates preferred in the context of the present invention are hydrates.

The inventive compounds may, depending on their structure, exist in different stereoisomeric forms, i.e. in the form of configurational isomers or else optionally as conformational isomers. The inventive compounds have a centre of asymmetry at the carbon atom of the diazepine skeleton (C-4) bonded to Y via —(CH₂)ₚ—. They may therefore take the form of pure enantiomers, racemates, or else of diastereomers or mixtures thereof when one or more of the substituents described in the formula (I) contains a further element of asymmetry, for example a chiral carbon atom. The present invention therefore also encompasses enantiomers and diastereomers, and the respective mixtures thereof. The pure enantiomers and diastereomers can be isolated from such mixtures in a known manner; for this purpose, preference is given to using chromatography processes, especially HPLC chromatography on a chiral or achiral phase.

In general, the inventive enantiomers inhibit the target to different degrees and have different activity in the cancer cell lines studied. The more active enantiomer, which frequently is the 4S enantiomer, is preferred.

Where the inventive compounds can occur in tautomeric forms, the present invention encompasses all the tautomeric forms.

The present invention also encompasses all suitable isotopic variants of the inventive compounds. An isotopic variant of an inventive compound is understood here to mean a compound in which at least one atom within the inventive compound has been exchanged for another atom of the same atomic number but with a different atomic mass from the atomic mass which usually or predominantly occurs in nature. Examples of isotopes which can be incorporated into a compound according to the invention are those of hydrogen, carbon, nitrogen, oxygen, phosphorus, sulphur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of a compound according to the invention, especially those in which one or more radioactive isotopes have been incorporated, may be beneficial, for example, for the examination of the mechanism of action or of the active ingredient distribution in the body; due to comparatively easy preparability and detectability, especially compounds labelled with ³H or ¹⁴C isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required; such modifications of the compounds according to the invention may therefore in some cases also constitute a preferred embodiment of the present invention. Isotopic variants of the inventive compounds can be prepared by the processes known to those skilled in the art, for example by the methods described below and the procedures described in the working examples, by using corresponding isotopic modifications of the respective reagents and/or starting compounds.

In addition, the present invention also encompasses prodrugs of the inventive compounds. The term “prodrugs” includes compounds which may themselves be biologically active or inactive but are converted to inventive compounds while resident in the body (for example metabolically or hydrolytically).

The inventive compounds can act systemically and/or locally. For this purpose, they can be administered in a suitable manner, for example by the oral, parenteral, pulmonary, nasal, sublingual, lingual, buccal, rectal, dermal, transdermal, conjunctival or otic route, or as implant or stent.

The inventive compounds can be administered in suitable administration forms for these administration routes.

Suitable administration forms for oral administration are those which function according to the prior art and deliver the inventive compounds rapidly and/or in modified fashion, and which contain the inventive compounds in crystalline and/or amorphized and/or dissolved form, for example tablets (uncoated or coated tablets, for example having enteric coatings or coatings which are insoluble or dissolve with a delay and control the release of the compound according to the invention), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Parenteral administration can be accomplished with avoidance of a resorption step (for example by an intravenous, intraarterial, intracardiac, intraspinal or intralumbar route) or with inclusion of a resorption (for example by an intramuscular, subcutaneous, intracutaneous, percutaneous or intraperitoneal route). Administration forms suitable for parenteral administration include preparations for injection and infusion in the form of solutions, suspensions, emulsions, lyophilizates or sterile powders.

Suitable administration forms for the other administration routes are, for example, pharmaceutical forms for inhalation (including powder inhalers, nebulizers), nasal drops, solutions or sprays; tablets for lingual, sublingual or buccal administration, films/wafers or capsules, suppositories, preparations for the ears or eyes, vaginal capsules, aqueous suspensions (lotions, shaking mixtures), lipophilic suspensions, ointments, creams, transdermal therapeutic systems (for example patches), milk, pastes, foams, dusting powders, implants or stents.

The inventive compounds can be converted to the administration forms mentioned. This can be accomplished in a manner known per se by mixing with inert, nontoxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), colourants (e.g. inorganic pigments, for example iron oxides) and flavour and/or odour correctants.

The present invention further provides medicaments comprising the inventive compounds, typically together with one or more inert, nontoxic, pharmaceutically suitable auxiliaries, and for the use thereof for the aforementioned purposes.

The inventive compounds are formulated to give pharmaceutical preparations in a manner known per se, by converting the active ingredient(s) to the desired administration form with the excipients customary in the pharmaceutical formulation.

The excipients used may, for example, be carrier substances, fillers, disintegrants, binders, humectants, glidants, absorbents and adsorbents, diluents, solvents, cosolvents, emulsifiers, solubilizers, taste correctors, colourants, preservatives, stabilizers, wetting agents, salts for modifying the osmotic pressure or buffers. Reference should be made to Remington's Pharmaceutical Science, 15th ed. Mack Publishing Company, East Pennsylvania (1980).

The pharmaceutical formulations can be present

in solid form, for example as tablets, sugar-coated tablets, pills, suppositories, capsules, transdermal systems or
in semisolid form, for example as ointments, creams, gels, suppositories, emulsions or
in liquid form, for example as solutions, tinctures, suspensions or emulsions.

Excipients in the context of the invention may, for example, be salts, saccharides (mono-, di-, tri-, oligo- and/or polysaccharides), proteins, amino acids, peptides, fats, waxes, oils, hydrocarbons and derivatives thereof, and the excipients may be of natural origin or be obtained by synthetic or partially synthetic means.

Useful forms for oral or peroral administration are especially tablets, sugar-coated tablets, capsules, pills, powders, granules, pastilles, suspensions, emulsions or solutions.

Useful forms for parenteral administration are especially suspensions, emulsions, and particularly solutions.

The present invention relates to the inventive compounds.

They can be used for the prophylaxis and therapy of human disorders, in particular neoplastic disorders.

The inventive compounds can be used in particular for inhibiting or reducing cell proliferation and/or cell division and/or to induce apoptosis.

The inventive compounds are suitable in particular for prophylaxis and/or treatment of hyperproliferative disorders, for example
psoriasis,
keloids and other skin hyperplasias,
benign prostate hyperplasias (BPH),
solid tumours and
haematological tumours.

Solid tumours that can be treated in accordance with the invention are, for example, tumours of the breast, the respiratory tract, the brain, the reproductive organs, the gastrointestinal tract, the urogenital tract, the eye, the liver, the skin, the head and the neck, the thyroid gland, the parathyroid gland, the bones, and the connective tissue and metastases of these tumours.

Haematological tumours which can be treated are, for example,
multiple myelomas
lymphomas or
leukaemias

Breast tumours which can be treated are, for example:
breast carcinomas with positive hormone receptor status
breast carcinomas with negative hormone receptor status
Her-2 positive breast carcinomas
hormone receptor and Her-2 negative breast carcinomas
BRCA—associated breast carcinomas
inflammatory breast carcinomas.

Tumours of the respiratory tract which can be treated are, for example,
non-small-cell bronchial carcinomas such as, for example, squamous cell carcinoma, adenocarcinoma, large-cell carcinoma and
small-cell bronchial carcinomas.

Tumours of the brain which can be treated are, for example,
gliomas,
glioblastomas,
astrocytomas,
meningiomas and
medulloblastomas.

Tumours of the male reproductive organs which can be treated are, for example:
prostate carcinomas,
malignant epididymal tumours
malignant testicular tumours and
penis carcinomas.

Tumours of the female reproductive organs which can be treated are, for example:
endometrial carcinomas
cervix carcinomas
ovarian carcinomas
vaginal carcinomas
vulvar carcinomas

Tumours of the gastrointestinal tract which can be treated are, for example:
colorectal carcinomas
anal carcinomas
stomach carcinomas
pancreas carcinomas
oesophagus carcinomas
gall bladder carcinomas
carcinomas of the small intestine
salivary gland carcinomas
neuroendocrine tumours
gastrointestinal stroma tumours

Tumours of the urogenital tract which can be treated are, for example:
urinary bladder carcinomas
kidney cell carcinomas
carcinomas of the renal pelvis and lower urinary tract

Tumours of the eye which can be treated are, for example:
retinoblastomas
intraocular melanomas

Tumours of the liver which can be treated are, for example:
hepatocellular carcinomas
cholangiocellular carcinomas

Tumours of the skin which can be treated are, for example:
malignant melanomas
basaliomas
spinaliomas
Kaposi sarcomas
Merkel cell carcinomas

Tumours of the head and neck which can be treated are, for example:
larynx carcinomas
carcinomas of the pharynx and the oral cavity
carcinomas of the middle line structures (e.g. NMC, C.A. French, Annu. Rev. Pathol. 2012, 7:247-265)

Sarcomas which can be treated are, for example:
soft tissue sarcomas
osteosarcomas

Lymphomas which can be treated are, for example:
non-Hodgkin lymphomas
Hodgkin lymphomas
cutaneous lymphomas
lymphomas of the central nervous system
AIDS-associated lymphomas

Leukaemias which can be treated are, for example:
acute myeloid leukaemias
chronic myeloid leukaemias
acute lymphatic leukaemias
chronic lymphatic leukaemias
hairy cell leukaemias

Advantageously, the inventive compounds can be used for prophylaxis and/or treatment of leukaemias, especially acute myeloid leukaemias, prostate carcinomas, especially androgen receptor-positive prostate carcinomas, cervical carcinomas, mammary carcinomas, especially hormone receptor-negative, hormone receptor-positive or BRCA-associated mammary carcinomas, pancreatic carcinomas, renal cell carcinomas, hepatocellular carcinomas, melanomas and other skin tumours, non-small-cell bronchial carcinomas, endometrial carcinomas and colorectal carcinomas.

Particularly advantageously, the inventive compounds can be used for prophylaxis and/or treatment of leukaemias, especially acute myeloid leukaemias, prostate carcinomas, especially androgen receptor-positive prostate carcinomas, mammary carcinomas, especially oestrogen receptor alpha-negative mammary carcinomas, melanomas or multiple myelomas.

The inventive compounds are also suitable for prophylaxis and/or treatment of benign hyperproliferative diseases, for example endometriosis, leiomyoma and benign prostate hyperplasia.

The inventive compounds are also suitable for male fertility control.

The inventive compounds are also suitable for prophylaxis and/or treatment of systemic inflammatory diseases, especially LPS-induced endotoxic shock and/or bacteria-induced sepsis.

The inventive compounds are also suitable for prophylaxis and/or treatment of inflammatory or autoimmune disorders, for example:
pulmonary disorders associated with inflammatory, allergic and/or proliferative processes: chronic obstructive pulmonary disorders of any origin, particularly bronchial asthma; bronchitis of different origin; all forms of restrictive pulmonary disorders, particularly allergic alveolitis; all forms of pulmonary oedema, particularly toxic pulmonary oedema; sarcoidoses and granulomatoses, particularly Boeck's disease,
rheumatic disorders/autoimmune disorders/joint disorders associated with inflammatory, allergic and/or proliferative processes: all forms of rheumatic disorders, especially rheumatoid arthritis, acute rheumatic fever, polymyalgia rheumatica; reactive arthritis; inflammatory soft-tissue disorders of other origin; arthritic symptoms in the case of degenerative joint disorders (arthroses); traumatic arthritides; collagenoses of any origin, e.g. systemic lupus erythematosus, scleroderma, polymyositis, dermatomyositis, Sjögren's syndrome, Still's syndrome, Felty's syndrome
allergies associated with inflammatory and/or proliferative processes: all forms of allergic reactions, e.g. angiooedema, hay fever, insect bites, allergic reactions to medicaments, blood derivatives, contrast agents, etc., anaphylactic shock, urticaria, contact dermatitis
vascular inflammation (vasculitis): panarteritis nodosa, temporal arteritis, erythema nodosum
dermatological disorders associated with inflammatory, allergic and/or proliferative processes: atopic dermatitis; psoriasis; pityriasis rubra pilaris; erythematous disorders triggered by different noxae, for example radiation, chemicals, burns, etc.; bullous dermatoses; lichenoid disorders; pruritus; seborrhoeic eczema; rosacea; pemphigus vulgaris; erythema exsudativum multiforme; balanitis; vulvitis; hair loss, such as alopecia areata; cutaneous T-cell lymphoma
renal disorders associated with inflammatory, allergic and/or proliferative processes: nephrotic syndrome; all nephritides
hepatic disorders associated with inflammatory, allergic and/or proliferative processes: acute hepatic disintegration; acute hepatitis of different origin, for example viral, toxic, medicament-induced; chronic aggressive and/or chronic intermittent hepatitis
gastrointestinal disorders associated with inflammatory, allergic and/or proliferative processes: regional enteritis (Crohn's disease); ulcerative colitis; gastritis; reflux oesophagitis; gastroenteritides of other origin, e.g. indigenous sprue
proctological disorders associated with inflammatory, allergic and/or proliferative processes: anal eczema; fissures; haemorrhoids; idiopathic proctitis
ocular disorders associated with inflammatory, allergic and/or proliferative processes: allergic keratitis, uveitis, iritis; conjunctivitis; blepharitis; optic neuritis; chlorioditis; sympathetic ophthalmia
disorders of the ear-nose-throat region associated with inflammatory, allergic and/or proliferative processes: allergic rhinitis, hay fever; otitis externa, for example caused by contact eczema, infection, etc.; otitis media
neurological disorders associated with inflammatory, allergic and/or proliferative processes:

cerebral oedema, particularly tumour-related cerebral oedema; multiple sclerosis; acute encephalomyelitis; meningitis; various forms of seizure, for example West's syndrome
haematological disorders associated with inflammatory, allergic and/or proliferative processes: acquired haemolytic anaemia; idiopathic thrombocytopenia
neoplastic disorders associated with inflammatory, allergic and/or proliferative processes: acute lymphatic leukaemia; malignant lymphomas; lymphogranulomatoses; lymphosarcoma; extensive metastases, particularly in the case of mammary, bronchial and prostate carcinoma
endocrine disorders associated with inflammatory, allergic and/or proliferative processes: endocrine orbitopathy; thyrotoxic crisis; de Quervain's thyroiditis; Hashimoto's thyroiditis; Basedow's disease
organ and tissue transplants, graft-versus-host disease
severe states of shock, for example anaphylactic shock, systemic inflammatory response syndrome (SIRS)
substitution therapy in the case of: congenital primary adrenal insufficiency, for example congenital adrenogenital syndrome; acquired primary adrenal insufficiency, for example Addison's disease, autoimmune adrenalitis, postinfectious, tumours, metastases, etc; congenital secondary adrenal insufficiency, for example congenital hypopituitarism; acquired secondary adrenal insufficiency, for example postinfectious, tumours, etc.
emesis associated with inflammatory, allergic and/or proliferative processes, for example in combination with a 5-HT3 antagonist in the case of cytostatic-induced vomiting
pain of inflammatory origin, for example lumbago.

The inventive compounds are also suitable for the treatment of viral disorders, for example infections caused by papilloma viruses, herpes viruses, Epstein-Barr viruses, hepatitis B or C viruses, and human immunodeficiency viruses.

The inventive compounds are also suitable for the treatment of atherosclerosis, dyslipidaemia, hypercholesterolaemia, hypertriglyceridaemia, peripheral vascular disorders, cardiovascular disorders, angina pectoris, ischaemia, stroke, myocardial infarction, angioplastic restenosis, hypertension, thrombosis, obesity, endotoxaemia.

The inventive compounds are also suitable for the treatment of neurodegenerative diseases, for example multiple sclerosis, Alzheimer's disease and Parkinson's disease.

These disorders are well characterized in man, but also exist in other mammals.

The present application further provides the inventive compounds for use as medicaments, especially for prophylaxis and/or treatment of neoplastic disorders.

The present application further provides the inventive compounds for prophylaxis and/or treatment of leukaemias, especially acute myeloid leukaemias, prostate carcinomas, especially androgen receptor-positive prostate carcinomas, cervical carcinomas, mammary carcinomas, especially hormone receptor-negative, hormone receptor-positive or BRCA-associated mammary carcinomas, pancreatic carcinomas, renal cell carcinomas, hepatocellular carcinomas, melanomas and other skin tumours, non-small-cell bronchial carcinomas, endometrial carcinomas and colorectal carcinomas.

The present application further provides the inventive compounds for prophylaxis and/or treatment of leukaemias, especially acute myeloid leukaemias, prostate carcinomas, especially androgen receptor-positive prostate carcinomas, mammary carcinomas, especially oestrogen receptor alpha-negative mammary carcinomas, melanomas or multiple myelomas.

The invention further provides for the use of the inventive compounds for production of a medicament.

The present application further provides for the use of the inventive compounds for production of a medicament for prophylaxis and/or treatment of neoplastic disorders.

The present application further provides for the use of the inventive compounds for production of a medicament for prophylaxis and/or treatment of leukaemias, especially acute myeloid leukaemias, prostate carcinomas, especially androgen receptor-positive prostate carcinomas, cervical carcinomas, mammary carcinomas, especially hormone receptor-negative, hormone receptor-positive or BRCA-associated mammary carcinomas, pancreatic carcinomas, renal cell carcinomas, hepatocellular carcinomas, melanomas and other skin tumours, non-small-cell bronchial carcinomas, endometrial carcinomas and colorectal carcinomas.

The present application further provides for the use of the inventive compounds for production of a medicament for prophylaxis and/or treatment of leukaemias, especially acute myeloid leukaemias, prostate carcinomas, especially androgen receptor-positive prostate carcinomas, mammary carcinomas, especially oestrogen receptor alpha-negative mammary carcinomas, melanomas or multiple myelomas.

The present application further provides for the use of the inventive compounds for prophylaxis and/or treatment of neoplastic disorders.

The present application further provides for the use of the inventive compounds for prophylaxis and/or treatment of leukaemias, especially acute myeloid leukaemias, prostate carcinomas, especially androgen receptor-positive prostate carcinomas, cervical carcinomas, mammary carcinomas, especially hormone receptor-negative, hormone receptor-positive or BRCA-associated mammary carcinomas, pancreatic carcinomas, renal cell carcinomas, hepatocellular carcinomas, melanomas and other skin tumours, non-small-cell bronchial carcinomas, endometrial carcinomas and colorectal carcinomas.

The present application further provides for the use of the inventive compounds for prophylaxis and/or treatment of leukaemias, especially acute myeloid leukaemias, prostate carcinomas, especially androgen receptor-positive prostate carcinomas, mammary carcinomas, especially oestrogen receptor alpha-negative mammary carcinomas, melanomas or multiple myelomas.

The present application further provides pharmaceutical formulations in the form of tablets comprising one of the inventive compounds for prophylaxis and/or treatment of leukaemias, especially acute myeloid leukaemias, prostate carcinomas, especially androgen receptor-positive prostate carcinomas, cervical carcinomas, mammary carcinomas, especially hormone receptor-negative, hormone receptor-positive or BRCA-associated mammary carcinomas, pancreatic carcinomas, renal cell carcinomas, hepatocellular carcinomas, melanomas and other skin tumours, non-small-cell bronchial carcinomas, endometrial carcinomas and colorectal carcinomas.

The present application further provides pharmaceutical formulations in the form of tablets comprising one of the inventive compounds for prophylaxis and/or treatment of leukaemias, especially acute myeloid leukaemias, prostate carcinomas, especially androgen receptor-positive prostate carcinomas, mammary carcinomas, especially oestrogen receptor alpha-negative mammary carcinomas, melanomas or multiple myelomas.

The invention further provides for the use of the inventive compounds for treatment of disorders associated with proliferative processes.

The invention further provides for the use of the inventive compounds for treatment of benign hyperplasias, inflammation disorders, autoimmune disorders, sepsis, viral infections, vascular disorders and neurodegenerative disorders.

The inventive compounds can be used alone or, if required, in combination with one or more other pharmacologically active substances, provided that this combination does not lead to undesirable and unacceptable side effects. The present invention therefore further provides medicaments comprising an inventive compound and one or more further active ingredients, especially for prophylaxis and/or treatment of the disorders mentioned above.

For example, the inventive compounds can be combined with known antihyperproliferative, cytostatic or cytotoxic substances for treatment of cancer. The combination of the inventive compounds with other substances commonly used for cancer treatment, or else with radiotherapy, is particularly appropriate.

An illustrative but nonexhaustive list of suitable combination active ingredients is as follows: abiraterone acetate, abraxane, acolbifene, Actimmune, actinomycin D (dactinomycin), afatinib, affinitak, Afinitor, aldesleukin, alendronic acid, alfaferone, alitretinoin, allopurinol, Aloprim, Aloxi, alpharadin, altretamine, aminoglutethimide, aminopterin, amifostine, amrubicin, amsacrine, anastrozole, anzmet, apatinib, Aranesp, arglabin, arsenic trioxide, Aromasin, arzoxifen, asoprisnil, L-asparaginase, atamestane, atrasentane, avastin, axitinib, 5-azacytidine, azathioprine, BCG or Tice BCG, bendamustine, bestatin, beta-methasone acetate, betamethasone sodium phosphate, bexarotene, bicalutamide, bleomycin sulphate, broxuridine, bortezomib, bosutinib, busulfan, cabazitaxel, calcitonin, campath, camptothecin, capecitabine, carboplatin, carfilzomib, carmustine, casodex, CCI-779, CDC-501, cediranib, cefesone, celebrex, celmoleukin, cerubidine, cediranib, chlorambucil, cisplatin, cladribine, clodronic acid, clofarabine, colaspase, corixa, crisnatol, crizotinib, cyclophosphamide, cyproterone acetate, cytarabine, dacarbazine, dactinomycin, dasatinib, daunorubicin, DaunoXome, Decadron, Decadron Phosphate, decitabine, degarelix, delestrogen, denileukin diftitox, depomedrol, deslorelin, dexrazoxane, diethylstilbestrol, diflucan, 2′,2′-difluorodeoxycytidine, DN-101, docetaxel, doxifluridine, doxorubicin (Adriamycin), dronabinol, dSLIM, dutasteride, DW-166HC, edotecarin, eflornithine, Eligard, Elitek, Ellence, Emend, enzalutamide, epirubicin, epoetin-alfa, Epogen, epothilone and derivatives thereof, eptaplatin, ergamisol, erlotinib, erythro-hydroxynonyladenine, estrace, oestradiol, oestramustine sodium phosphate, ethinyloestradiol, Ethyol, etidronic acid, etopophos, etoposide, everolimus, exatecan, exemestane, fadrozole, farston, fenretinide, filgrastim, finasteride, fligrastim, floxuridine, fluconazole, fludarabine, 5-fluorodeoxyuridine monophosphate, 5-fluorouracil (5-FU), fluoxymesterone, flutamide, folotin, formestane, fosteabine, fotemustine, fulvestrant, Gammagard, gefitinib, gemcitabine, gemtuzumab, Gleevec, Gliadel, goserelin, gossypol, granisetrone hydrochloride, hexamethylmelamine, histamine dihydrochloride, histrelin, holmium-166-DOTPM, hycamtin, hydrocortone, erythro-hydroxynonyladenine, hydroxyurea, hydroxyprogesterone caproate, ibandronic acid, ibritumomab tiuxetan, idarubicin, ifosfamide, imatinib, iniparib, interferon-alpha, interferon-alpha-2, interferon-alpha-2α, interferon-alpha-2β, interferon-alpha-n1, interferon-alpha-n3, interferon-beta, interferon-gamma-1α, interleukin-2, intron A, iressa, irinotecan, ixabepilone, keyhole limpet haemocyanin, kytril, lanreotide, lapatinib, lasofoxifene, lentinan sulphate, lestaurtinib, letrozole, leucovorin, leuprolide, leuprolide acetate, levamisole, levofolic acid calcium salt, levothroid, levoxyl, Libra, liposomal MTP-PE, lomustine, lonafarnib, lonidamine, marinol, mechlorethamine, mecobalamine, medroxyprogesterone acetate, megestrol acetate, melphalan, Menest, 6-mercaptopurine, mesna, methotrexate, metvix, miltefosine, minocycline, minodronate, miproxifen, mitomycin C, mitotan, mitoxantrone, modrenal, MS-209, MX-6, myocet, nafarelin, nedaplatin, nelarabine, nemorubicin, neovastat, neratinib, neulasta, neumega, neupogen, nilotimib, nilutamide, nimustine, nolatrexed, nolvadex, NSC-631570, obatoclax, oblimersen, OCT-43, octreotide, olaparib, ondansetron hydrochloride, Onco-TCS, Orapred, Osidem, oxaliplatin, paclitaxel, pamidronate disodium, pazopanib, pediapred, pegaspargase, pegasys, pemetrexed, pentostatin, N-phosphonoacetyl-L-aspartate, picibanil, pilocarpine hydrochloride, pirarubicin, plerixafor, plicamycin, PN-401, porfimer sodium, prednimustine, prednisolone, prednisone, Premarin, procarbazine, Procrit, QS-21, quazepam, R-1589, raloxifene, raltitrexed, ranpirnas, RDEA119, Rebif, regorafenib, 13-cis-retinoic acid, rhenium-186 etidronate, rituximab, roferon-A, romidepsin, romurtide, ruxolitinib, salagen, salinomycin, sandostatin, sargramostim, satraplatin, semaxatinib, semustine, seocalcitol, sipuleucel-T, sizofiran, sobuzoxan, Solu-Medrol, sorafenib, streptozocin, strontium-89 chloride, sunitinib, Synthroid, T-138067, tamoxifen, tamsulosin, Tarceva, tasonermin, tastolactone, Taxoprexin, Taxoter, teceleukin, temozolomide, temsirolimus, teniposide, testosterone propionate, Testred, thalidomide, thymosin alpha-1, thioguanine, thiotepa, thyrotropin, tiazorufin, tiludronic acid, tipifarnib, tirapazamine, TLK-286, toceranib, topotecan, toremifen, tositumomab, tastuzumab, teosulfan, transMID-107R, tretinoin, Trexall, trimethylmelamine, trimetrexate, triptorelin acetate, triptorelin pamoate, trofosfamide, UFT, uridine, valrubicin, valspodar, vandetanib, vapreotide, vatalanib, vemurafinib, verte-porfin, vesnarinone, vinblastine, vincristine, vindesine, vinflumine, vinorelbine, virulizin, vismodegib, Xeloda, Z-100, Zinecard, zinostatin stimalamer, zofran, zoledronic acid.

The combination of the inventive compound with a P-TEFb or CDK9 inhibitor is likewise particularly appropriate.

In a very promising manner, the inventive compounds can also be combined with biologics such as antibodies (for example aflibercept, alemtuzumab, bevacizumab, brentuximumab, catumaxomab, cetuximab, denosumab, edrecolomab, gemtuzumab, ibritumomab, ipilimumab, ofatumumab, panitumumab, pertuzumab, rituximab, tositumumab, trastuzumab) and recombinant proteins.

The inventive compounds can also achieve positive effects in combination with other therapies directed against angiogenesis, for example with bevacizumab, axitinib, regorafenib, cediranib, sorafenib, sunitinib or thalidomide. Combinations with antihormones and steroidal metabolic enzyme inhibitors are particularly suitable because of their favourable profile of side effects.

Generally, the following aims can be pursued with the combination of the inventive compounds with other cytostatically or cytotoxically active agents:
improved efficacy in slowing the growth of a tumour, in reducing its size or even in the complete elimination thereof, compared with treatment with an individual active ingredient;
the possibility of using the chemotherapeutics used in a lower dosage than in the case of monotherapy;
the possibility of a more tolerable therapy with fewer side effects compared with individual administration;
the possibility of treatment of a broader spectrum of tumour diseases;
the achievement of a higher rate of response to the therapy;
a longer survival time of the patient compared with present-day standard therapy.

In addition, the inventive compounds can also be used in conjunction with radiotherapy and/or surgical intervention.

### Preparation of the Inventive Compounds

The preparation of the compounds of the general formula (I) is described in an exemplary manner by the schemes below:

4-Aminopyrroloacetophenones can be prepared by a reaction sequence shown in Scheme 1.

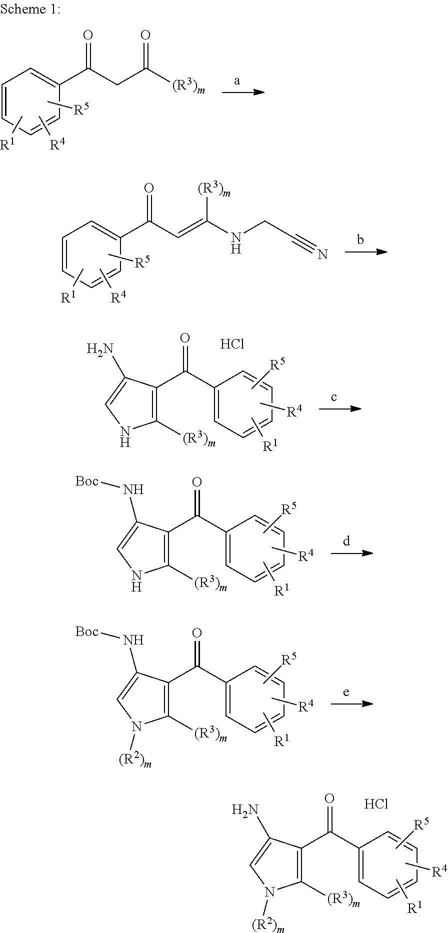

Here, R¹, R², R³, R⁴ and R⁵ as well as n and m have the meanings given under the General Formula I.

a) 2-aminoacetonitrile, base, solvent, reflux, removal of water; b) EtOH, base, then HCl*dioxane; c) Boc₂O, base; d) R² LG, base, opt. catalyst; e) e.g. HCl*dioxane

The reaction sequence a) and b) for cyclizing the pyrrole is a sequence known to the person skilled in the art (Il Farmaco, Edizione Scientifica (1984), 39, p. 538ff, Tarzia et al.). By reaction with corresponding alkyl halides or alkyl sulphates in Step d), it is possible to introduce alkyl substituents R² in accordance with the general formula (I) using methods known to the person skilled in the art. By reaction with acyl halides or acyl anhydrides or aryl- and alkylsulphonyl chlorides, it is possible to introduce acyl or aryl- or alkylsulphonyl substituents as R² as per the general formula (I) by methods known to the person skilled in the art. Aryl and heteroaryl radicals as R² can be introduced by reaction with the corresponding aryl or heteroaryl halides and a palladium or copper transition metal catalyst (J. Am. Chem. Soc. (1998), 120, pp. 827-8, Hartwig et al.; Bioorg. Med Chem. Lett. (2011), 21, p. 4306ff, Xie et al.). LG should be understood here to mean a leaving group which, as described herein, may, for example, be a halogen or a boronic acid.

4-Aminopyrazoloacetophenones can be prepared by a reaction sequence shown in Scheme 2.

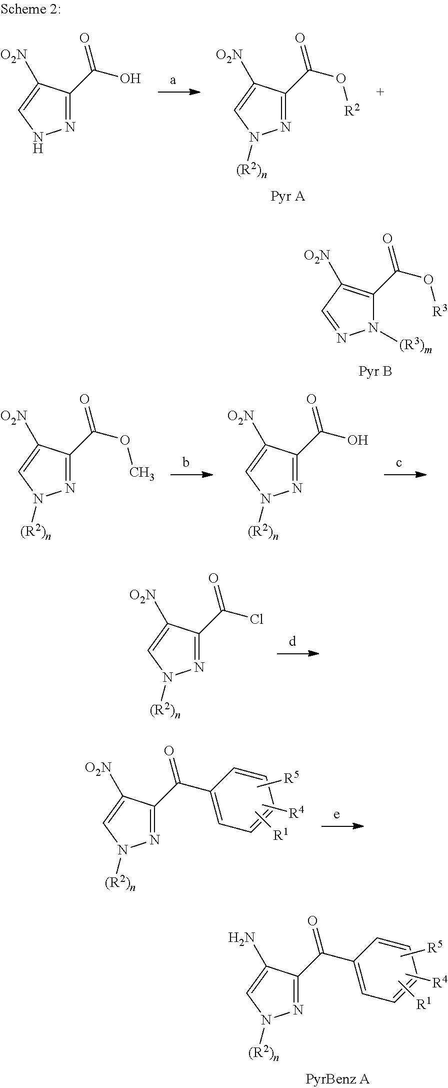

R¹, R², R³, R⁴ and R⁵, and also n and m, here are each as defined for the general formula I.

a) R² halogen, K₂CO₃, DMF; b) NaOH, MeOH, water; c) oxalyl chloride, POCl₃ or PCl₃; d) ArR¹R⁴R⁵, AlCl₃; e) Fe, NH₄Cl, water, EtOH

The reaction sequence a) to e) in Scheme 2 has been described (J Med. Chem. (1973), 16, p. 1346ff, DeWald et al.) and can be conducted analogously. By reaction with corresponding alkyl halides or alkyl sulphates in step a), it is possible to introduce alkyl substituents R² as per general formula (I) by methods known to the person skilled in the art. By reaction with acyl halides or acyl anhydrides or aryl- and alkylsulphonyl chlorides, it is possible to introduce acyl or aryl- or alkylsulphonyl substituents as R² as per general formula (I) by methods known to the person skilled in the art. Aryl and heteroaryl radicals as R² can be introduced by reaction with the corresponding aryl or heteroaryl halides and a palladium or copper transition metal catalyst (J. Am. Chem. Soc. (1998), 120, pp. 827-8, Hartwig et al.; Bioorg. Med Chem. Lett. (2011), 21, p. 4306ff, Xie et al.). LG should be understood here to mean a leaving group which, as described herein, may, for example, be a halogen or a boronic acid.

The pyrazoles Pyr A and Pyr B generated in reaction a) are converted separately to PyrBenz A and PyrBenz B by the reaction sequence according to Scheme 2.

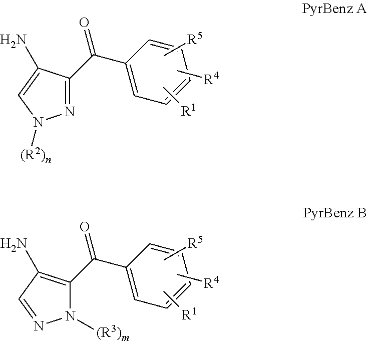

R¹, R², R³, R⁴ and R⁵, and also n and m, here are each as defined for the general formula I.

The diazepine ring in the pyrrolo- and pyrazolobenzophenones described is formed as described in generic terms in Scheme 3.

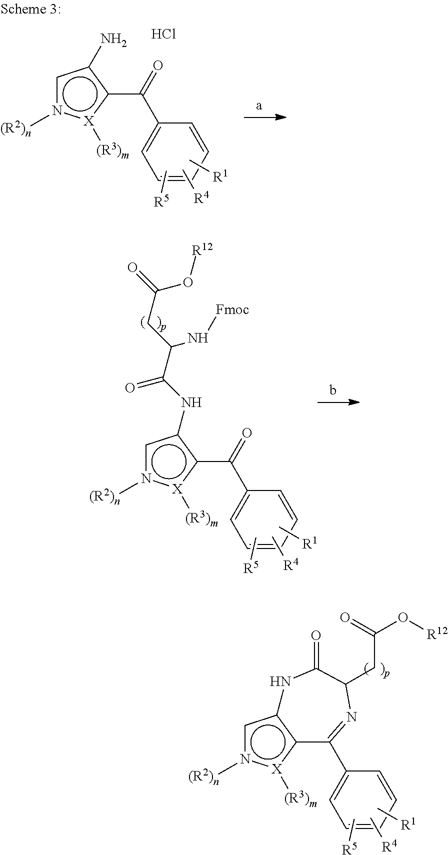

R¹, R², R³, R⁴, R⁵, R¹² and X, and also n, m and p, here are each as defined for the general formula I. In the formulae shown here for the intermediates and for the general formula I, a circle means the presence of possible double bond isomers in the case that X is nitrogen, as shown below:

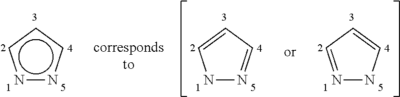

a) e.g. HATU, FMOC-ASP(OR¹²)—OH; b) e.g. piperidine, RT, then HOAc excess

Coupling a) is shown here with HATU, but can also be effected under other conditions. For this purpose, a multitude of methods compiled in appropriate reference books such as “Compendium of Organic Synthetic Methods”, volume I-VI (Wiley Interscience) or “The Practice of Peptide Synthesis”, Bodansky (Springer Verlag) are available to the person skilled in the art.

What is shown here is the use of the Fmoc protecting group on the amine of the amino acid used. It is also possible to work with other protecting groups, for example Boc. In that case, a strong acid such as trifluoroacetic acid or hydrochloric acid is used in step b).

Subsequently, the triazole ring is formed as described in Scheme 4. This affords inventive structures of the general formula I with Y═C(O)OR¹².

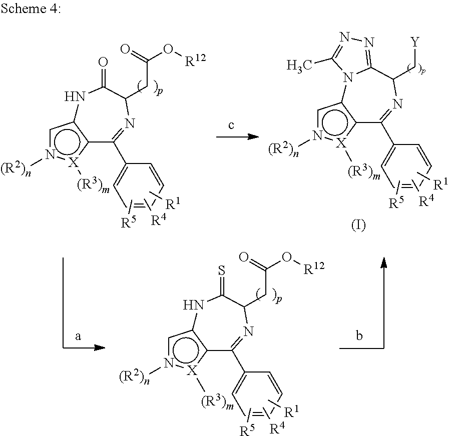

R¹, R², R³, R⁴, R⁵, R¹² and X, and also n, m and p, here are each as defined for the general formula I. Y is —C(O)OR¹² as per the definition of the general formula I. In the formulae shown here for the intermediates and for the general formula I, a circle means the presence of possible double bond isomers in the case that X is nitrogen, as shown below:

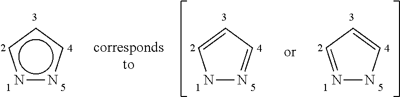

a) Lawesson's reagent, THF, reflux; b) AcNHNH₂, 1-BuOH, reflux; c) NaH, (EtO)₂P(O)Cl or (morpholino)₂P(O)Cl, THF, then AcNHNH₂, 1-BuOH, reflux;

There are further methods for forming the triazole ring (J. Heterocyclic Chem. (1979), 16, p. 793 ff, Moffett et al.; J. Med. Chem. (1980), 23, p. 392 ff Hester et al.). The reagents and solvents described in Scheme 4 are likewise mentioned merely as examples and can be replaced by similar reagents.

The inventive compounds of the general formula I with Y═C(O)NR¹⁰R¹¹ are prepared as described in Scheme 5.

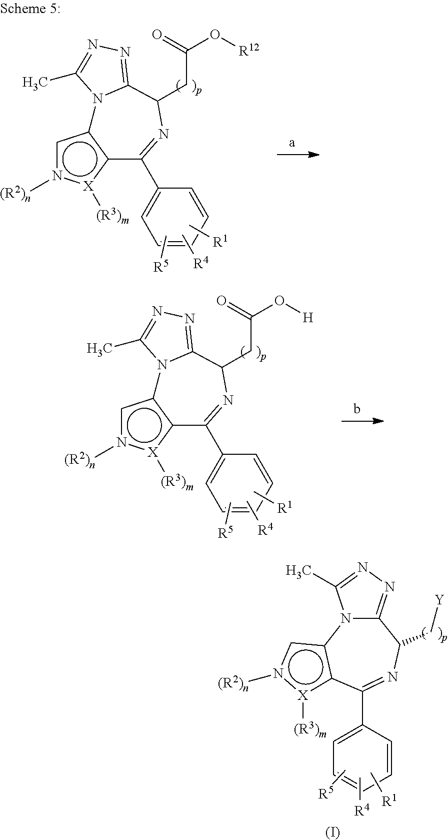

R¹, R², R³, R⁴, R⁵, R¹², X, n, m and p here are each as defined for the general formula I. Y is —C(O)NR¹⁰R¹¹ as per the definition of the general formula I. In the formulae shown here for the intermediates and for the general formula I, a circle means the presence of possible double bond isomers in the case that X is nitrogen, as shown below:

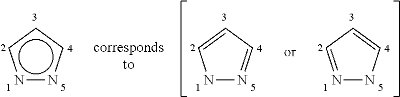

a) NaOH, MeOH, water; b) amine, HATU, base;

According to the nature of the ester, the reaction is effected under basic conditions, or else under acidic conditions.

Alkyl groups preferred in this context are methyl, ethyl or longer homologous esters. The reactions can preferably be performed using alkali metal hydroxides such as lithium hydroxide, sodium hydroxide or potassium hydroxide in aqueous alcoholic solutions. Branched alkyl groups such as tert-butyl esters can preferably be hydrolysed under acidic conditions. The person skilled in the art is aware of a multitude of methods. For illustrative purposes, mention is made here merely of the use, for example, of HCl in organic solvents or pure or dilute trifluoroacetic acid.

The inventive amides of the general formula I are thus prepared by reacting the carboxylic acids for example with the generally commercially available amines specified in the working examples, with additional activation by a method commonly known to those skilled in the art. Possible methods mentioned here are the use of HATU, HBTU, PyBOB or T3P with addition of a suitable base. The conversion of the carboxylic acids to their amides is described in general terms in reference books such as “Compendium of Organic Synthetic Methods”, volume I-VI (Wiley Interscience) or “The Practice of Peptide Synthesis”, Bodansky (Springer Verlag).

The inventive compounds of the general formula I with Y═C(O)R¹³ are prepared as described in Scheme 6.

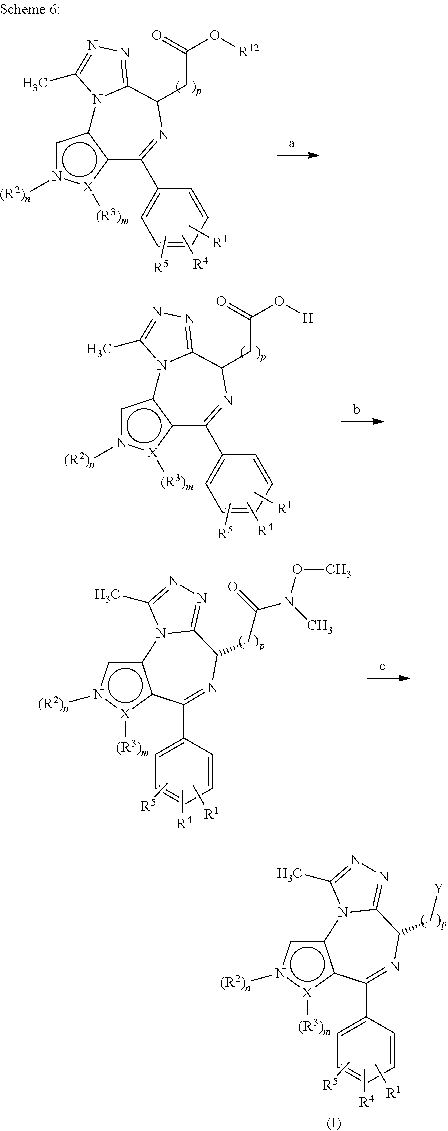

R¹, R², R³, R⁴, R⁵, R¹², X and Y, and also n, m and p, here are each as defined for the general formula I. Y is —C(O)R¹³ as per the definition of the general formula I. In the formulae shown here for the intermediates and for the general formula I, a circle means the presence of possible double bond isomers in the case that X is nitrogen, as shown below:

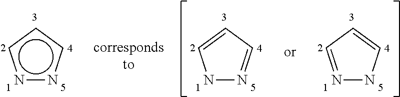

a) NaOH, MeOH, water; or TFA, CH₂Cl₂ b) N,O-dimethylhydroxylamine, HATU, base; c) Y—MgBr, THF

In the case of formation of such a carbon bond, in step b) the coupling to give a Weinreb amide known to the person skilled in the art is conducted by reaction with N,O-dimethylhydroxylamine. In step c), for example, an alkylmagnesium (Grignard) or alkyllithium reagent known to the person skilled in the art is then used for conversion to compounds of the general formula (I). The preparation of such alkylmagnesium or alkyllithium reagents is generally known to the person skilled in the art and can be conducted proceeding from corresponding alkyl halides such as iodides, bromides or chlorides with, for example, the elemental metal, for example magnesium or lithium, or else by reaction with a correspondingly reactive alkylmagnesium or alkyllithium reagent such as diisopropylmagnesium or butyllithium.

The inventive compounds of the general formula I with Y=phenyl or heteroaryl are prepared as described in Scheme 7.

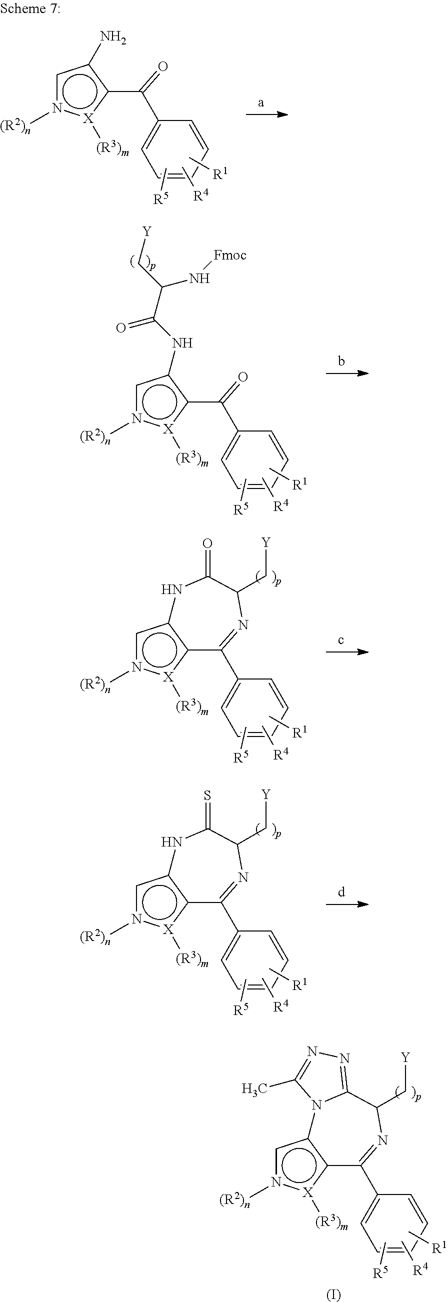

R¹, R², R³, R⁴, R⁵ and X, and also n, m and p, here are each as defined for the general formula I. Y is phenyl or heteroaryl as per the definition of the general formula I. In the formulae shown here for the intermediates and for the general formula I, a circle means the presence of possible double bond isomers in the case that X is nitrogen, as shown below:

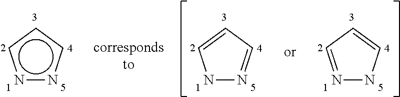

a) HATU, THF, FMOC-Ala(Y)—OH; b) piperidine, THF, RT, then HOAc excess; c) Lawesson's reagent, THF, reflux; d) AcNHNH₂, 1-BuOH, reflux;

A number of further amino acids having corresponding Y substitution as per general formula I are commercially available. These amino acids can be inserted by analogy, as known to those skilled in the art, into the structure formation to give compounds of the general formula (I).

The inventive compounds of the general formula I with Y=heteroaryl having 5 ring atoms are also prepared as described in Scheme 8, Part A and Part B.

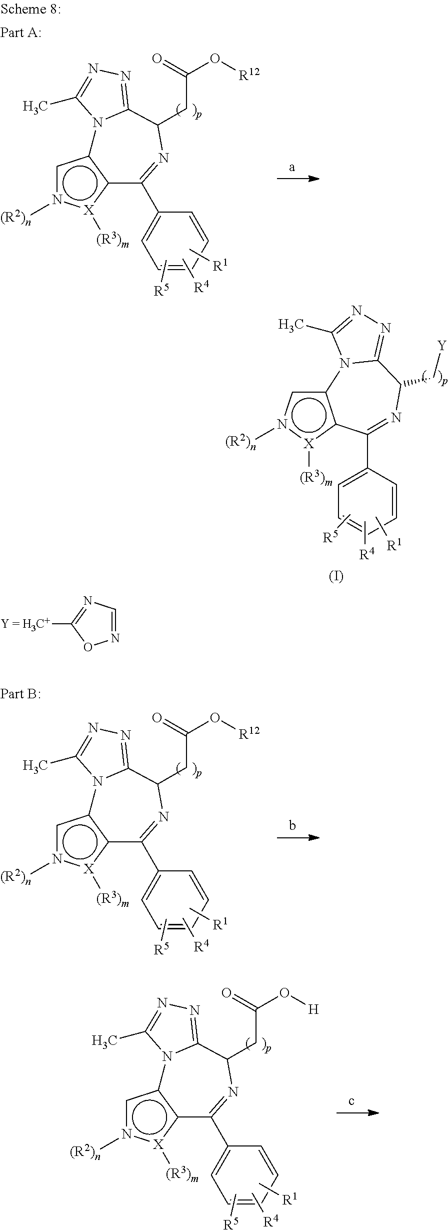

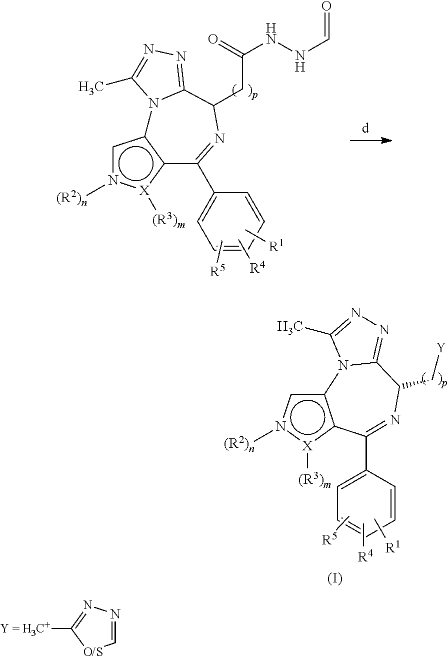

R¹, R², R³, R⁴, R⁵, R¹² and X, and also n, m and p, here are each as defined for the general formula I. Y is heteroaryl having 5 ring atoms as per the definition of the general formula I. In the formulae shown here for the intermediates and for the general formula I, a circle means the presence of possible double bond isomers in the case that X is nitrogen, as shown below:

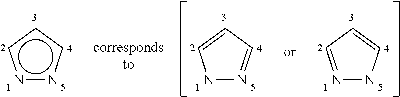

a) hydroxyamidine, NMP, 70° C., b) NaOH, MeOH, water; c) hydrazide, HATU, base; d) POCl₃ or Lawesson's reagent

The direct reaction of an ester with hydroxyamidines (Scheme 8, Part A) is described in the literature (Tetrahedron Lett. (2006), 47, p 4271-4, W. Du et al.). By this process, it is possible to convert either aliphatically substituted hydroxyamidines or aromatically substituted hydroxyamidines. Other heterocycles can be formed, by way of example, as described in steps b to d (Scheme 8, Part B). Using reactions known to those skilled in the art for elimination of water, for example phosphorus oxychloride, thionyl chloride, p-toluenesulphonyl chloride or Burgess' reagent, such a sequence leads, for example, to 1,3,4-oxadiazoles (J. Med. Chem. (2005), 48, p 4068 ff. Garcia et al.) or, using Lawesson's reagent or phosphorus pentasulphide, to 1,3,4-thiadiazoles (Eur. J. Med. Chem. (2010), 45, p 4664 ff. Kumar et al.) which may optionally bear different further substituents.

### ABBREVIATIONS

Asp aspartic acid
Boc tert-butoxycarbonyl
Boc-anhydride di-tert-butyl dicarbonate (CAS 24424-99-5)
CDCl₃ deuterochloroform
CHAPS 3-{dimethyl[3-(4-{5,9,16-trihydroxy-2,15-dimethyltetracyclo-[8.7.0.0^{2,7}.0^{11,15}]heptadecan-14-yl}pentanamido)propyl]azaniumyl}propane-1-sulphonate
CO₂ carbon dioxide
d day
DMF dimethylformamide
DMSO dimethyl sulphoxide
Fmoc fluoren-9-ylmethoxycarbonyl
h hour
HATU (7-aza-1H-benzotriazol-1-yl)-1,1,3,3-tetramethyluronium hexafluorophosphate
HBTU 2-(1H-benzotriazol-1-yl)-1,1,3,3-tetramethyluronium hexafluorophosphate
UPLC high-pressure, high-performance liquid chromatography
KOtBu potassium tert-butoxide
LC-MS liquid chromatography-coupled mass spectrometry
min minutes
NaH sodium hydride
NMR nuclear magnetic resonance spectroscopy
PyBOB benzotriazol-1-yloxytris(dimethylamino)phosphonium hexafluorophosphate
RP-HPLC reversed phase HPLC
RT room temperature
Rₜ retention time (in HPLC)
SFC supercritical fluid chromatography
T3P propylphosphonic anhydride
TFA trifluoroacetic acid
THF tetrahydrofuran

NMR signals are reported with their respective recognizable multiplicities or combinations thereof. In this context, s=singlet, d=doublet, t=triplet, q=quartet, qi=quintet, m=multiplet, b=broad signal. Signals having combined multiplicities are reported, for example, as dd=doublet of doublets.

For chromatography on silica gel, silica gel having a particle size of 40-63 μm, pre-packed in Biotage (KP-Sil) columns, was usually employed.

### Preparation of the Intermediates Employed for the Inventive Compounds

The examples which follow describe the preparation of the intermediates used with preference for preparation of the inventive compounds.

### Intermediate 1A

### 2-{[4-(4-Chlorophenyl)-4-oxobut-2-en-2-yl]amino}acetonitrile

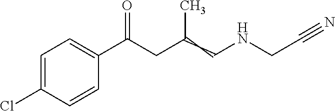 [SMILES: CC(=CNCC#N)CC(=O)c1ccc(Cl)cc1]

29.9 g of sodium bicarbonate were added to a suspension of 32.9 g of 2-aminoacetonitrile hydrochloride (CAS 6011-14-9) in 680 ml of ethanol. After 10 min of stirring at room temperature, 63.9 g of 1-(4-chlorophenyl)butane-1,3-dione (CAS 6302-55-2) and then 300 ml of toluene were added. The mixture was boiled at a Dean-Stark apparatus for 8 hours and conversion was checked by thin-layer chromatography. The mixture was cooled to room temperature, resulting in the formation of a strong precipitate. The mixture was diluted with water and ethyl acetate and extracted three times with ethyl acetate. The combined organic phases were washed with water, dried over sodium sulphate and concentrated under reduced pressure. The residue was subjected to fractional crystallization from methanol. This gave a total of 66.8 g of the desired 2-{[4-(4-chlorophenyl)-4-oxobut-2-en-2-yl]amino}acetonitrile.

¹H NMR (300 MHz, RT, CDCl₃): δ=2.21 (s, 3H); 4.22 (d, 2H); 5.84 (s, 1H); 7.38 (d, 2H); 7.79 (d, 2H); 11.32 (bs, 1H).

### Intermediate 1B

### 4-Amino-2-methyl-1H-pyrrol-3-yl 4-chlorophenyl ketone hydrochloride

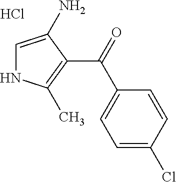 [SMILES: Cl ; Cc1[nH]cc(N)c1C(=O)c1ccc(Cl)cc1]

8.1 g of sodium ethoxide were added to a suspension of 27.3 g of Intermediate lA in 221 ml of ethanol, and the mixture was then stirred at RT for 30 min. Disappearance of the starting material was monitored by thin-layer chromatography. 63 ml of HCl in dioxane (4 M) were added and the mixture was stirred for 30 min. 500 ml of diethyl ether were then added, the mixture was stirred and the solid was filtered off with suction. This gave 37 g of the desired 4-amino-2-methyl-1H-pyrrol-3-yl4-chlorophenyl ketone hydrochloride.

¹H NMR (300 MHz, RT, DMSO-d6): δ=1.89 (s, 3H); 6.92 (d, 1H); 7.54 (d, 2H); 7.61 (d, 2H); 9.82 (bs, 2.5H); 11.82 (s, 1H).

### Intermediate 1C

### tert-Butyl N-[4-(4-chlorobenzoyl)-5-methyl-1H-pyrrol-3-yl]carbamate

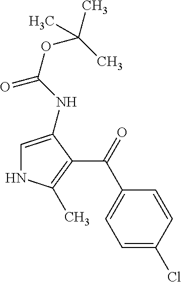 [SMILES: Cc1[nH]cc(NC(=O)OC(C)(C)C)c1C(=O)c1ccc(Cl)cc1]

At 0° C., 14.3 g of sodium carbonate were added to a solution of 36.5 g of Intermediate 1B and 29.4 g of Boc anhydride in 730 ml of dichloromethane. The cooling bath was removed and the mixture was stirred at room temperature for 6 hours. The reaction was monitored by thin-layer chromatography. A further 29.4 g of Boc anhydride and 10 ml of triethylamine were added and the mixture was stirred for one hour. The mixture was added to water and extracted three times with dichloromethane and the extracts were dried over sodium sulphate and concentrated under reduced pressure (1 mbar) on a rotary evaporator. The solution that remained was digested with pentane and the solid formed was filtered off with suction. This gave 29.1 g of tert-butyl N-[4-(4-chlorobenzoyl)-5-methyl-1H-pyrrol-3-yl]carbamate.

¹H NMR (300 MHz, RT, CDCl₃): δ=1.48 (s, 9H); 1.89 (s, 3H); 7.07 (s, 1H); 7.39 (d, 2H); 7.50 (d, 2H); 8.75 (bs, 1H); 8.9 (bs, 1H).

### Intermediate 1D

### tert-Butyl N-[4-(4-chlorobenzoyl)-1,5-dimethyl-1H-pyrrol-3-yl]carbamate

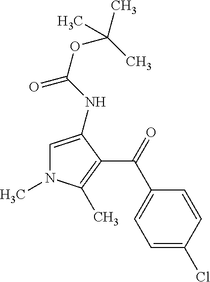 [SMILES: Cc1c(C(=O)c2ccc(Cl)cc2)c(NC(=O)OC(C)(C)C)cn1C]

At RT, 6.84 g of KOtBu were added to a solution of 20 g of Intermediate 1C in 160 ml of THF. The mixture was stirred for 10 min, 3.8 ml of iodomethane were then added dropwise and the mixture was stirred at RT for 4 h. The mixture was added to ice-water and extracted three times with ethyl acetate. The combined organic phases were washed with saturated sodium chloride solution and dried over sodium sulphate, and the solvent was removed under reduced pressure. The residue was taken up in dichloromethane and hexane was added, resulting in the precipitation of the desired product, which was filtered off with suction: 13.5 g. The mother liquor, which contained more product, was purified by chromatography on silica gel, giving a further 3.2 g of the desired tert-butyl N-[4-(4-chlorobenzoyl)-1,5-dimethyl-1H-pyrrol-3-yl]carbamate.

¹H NMR (300 MHz, RT, CDCl₃): δ=1.49 (s, 9H); 1.85 (s, 3H); 3.47 (s, 3H); 7.07 (s, 1H); 7.41 (d, 2H); 7.52 (d, 2H); 8.73 (bs, 1H).

### Intermediate 1E

### 4-Amino-1,2-dimethyl-1H-pyrrol-3-yl 4-chlorophenyl ketone hydrochloride

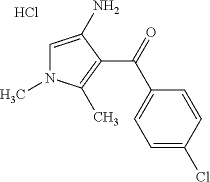 [SMILES: Cl ; Cc1c(C(=O)c2ccc(Cl)cc2)c(N)cn1C]

A solution of 14.6 g of Intermediate 1D in 157 ml of HCl in dioxane solution (4 M) was stirred at room temperature for 4 hours. The solution was stirred into 21 of methyl tert-butyl ether, resulting in the crystallization of the product. Filtration gave 10.1 g of 4-amino-1,2-dimethyl-1H-pyrrol-3-yl 4-chlorophenyl ketone hydrochloride.

¹H NMR (300 MHz, RT, CDCl₃): δ=1.88 (s, 3H); 3.53 (s, 3H); 7.05 (2, 1H); 7.56 (d, 2H); 7.62 (d, 2H); 9.88 (bs, 2H).

### Intermediate 1F

### 2-(3S)-[5-(4-Chlorophenyl)-6,7-dimethyl-2-oxo-1,2,3,7-tetrahydropyrrolo[3,4-e][1,4]diazepin-3-yl]acetic acid methyl ester

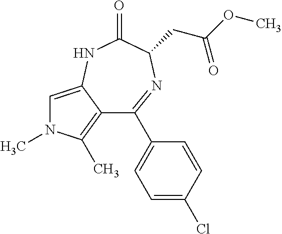

Under argon and at room temperature, a solution of 10.1 g of Intermediate 1E, 13.1 g of Fmoc-L-Asp(OMe)-OH (CAS 145038-53-5), 6.2 ml of diisopropylethylamine and 13.5 g of HATU in 144 ml of THF was stirred for 14 h. The mixture was partitioned between water and dichloromethane, the organic phase was removed and the aqueous phase was once more extracted with dichloromethane. The combined organic phases were washed with water and saturated sodium chloride solution, dried over sodium sulphate and concentrated. This gave methyl N-[4-(4-chlorobenzoyl)-1,5-dimethyl-1H-pyrrol-3-yl]-N²-[(9H-fluoren-9-ylmethoxy)carbonyl]-L-alpha-aspartate which was dissolved in 220 ml of THF. 19 g of piperidine were then added and the mixture was stirred at RT for 4.5 h. Subsequently, 76 ml of glacial acetic acid were added and the mixture was stirred for a further 14 h. The mixture was added to water and extracted three times with dichloromethane and the extracts were washed with saturated sodium chloride solution, dried over sodium sulphate and concentrated under reduced pressure. The crude product obtained was purified by chromatography on silica gel (hexane/ethyl acetate gradient). This gave 7.1 g of methyl 2-(3S)-[5-(4-chlorophenyl)-6,7-dimethyl-2-oxo-1,2,3,7-tetrahydropyrrolo[3,4-e][1,4]diazepin-3-yl]acetate.

¹H NMR (300 MHz, RT, CDCl₃): δ=1.79 (s, 3H); 3.12 (dd, 1H); 3.40 (dd, 1H); 3.53 (s, 3H); 3.73 (s, 3H); 4.40 (t, 1H); 6.45 (s, 1H); 7.32 (d, 2H); 7.47 (d, 2H); 7.97 (s, 1H).

### Intermediate 2A

### 2-[(4-Oxo-4-phenylbut-2-en-2-yl)amino]acetonitrile

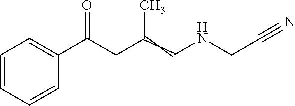 [SMILES: CC(=CNCC#N)CC(=O)c1ccccc1]

17.9 g of sodium bicarbonate were added to a suspension of 19.7 g of 2-aminoacetonitrile hydrochloride (CAS 6011-14-9) in 394 ml of ethanol. After 10 min of stirring at RT, 31.4 g of 1-phenylbutane-1,3-dione (CAS 93-91-4) and then 197 ml of toluene were added. The mixture was boiled at a Dean-Stark apparatus for 20 h and conversion was checked by thin-layer chromatography. The mixture was cooled to room temperature, resulting in the formation of a strong precipitate. The mixture was diluted with water and dichloromethane and extracted three times with dichloromethane. The combined organic phases were washed with water, dried over sodium sulphate and concentrated under reduced pressure. The residue was subjected to fractional crystallization from methanol. This gave a total of 32.6 g of the desired 2-[(4-oxo-4-phenylbut-2-en-2-yl)amino]acetonitrile.

¹H NMR (300 MHz, RT, CDCl₃): δ=2.20 (s, 3H); 4.22 (d, 2H); 5.89 (s, 1H); 7.39-7.50 (m, 3H); 7.84-7.89 (m, 2H); 11.33 (bs, 1H).

### Intermediate 2B

### 4-Amino-2-methyl-1H-pyrrol-3-yl phenyl ketone hydrochloride

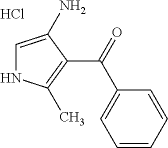

4 g of sodium methoxide were added to a suspension of 11.4 g of Intermediate 2A in 108 ml of ethanol (exothermic), and the mixture was stirred at RT for 15 min. Disappearance of the starting material was monitored by thin-layer chromatography. 28.5 ml of HCl in dioxane (4 M) were added and the mixture was stirred for 30 min. 110 ml of diethyl ether were then added and the resulting solid was filtered off with suction. This gave 12.8 g of the desired 4-amino-2-methyl-1H-pyrrol-3-yl phenyl ketone hydrochloride.

¹H NMR (300 MHz, RT, DMSO-d6): δ=1.89 (s, 3H); 6.94 (d, 1H); 7.50-7.56 (m, 2H); 7.58-7.64 (m, 3H); 9.81 (bs, 2.5H); 11.74 (s, 1H).

### Intermediate 2C

### tert-Butyl N-(4-benzoyl-5-methyl-1H-pyrrol-3-yl)carbamate

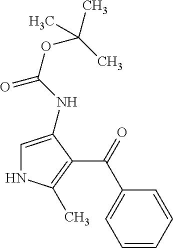 [SMILES: Cc1[nH]cc(NC(=O)OC(C)(C)C)c1C(=O)c1ccccc1]

At 0° C., 3.6 g of sodium carbonate were added to 7.5 g of Intermediate 2B and 6.9 g of Boc anhydride in 171 ml of dichloromethane. The mixture was gradually warmed to RT and stirred for 5 h. The mixture was added to water and extracted with dichloromethane and the extracts were washed with water and saturated sodium chloride solution, dried over sodium sulphate and concentrated under reduced pressure. This gave 4.4 g of tert-butyl N-(4-benzoyl-5-methyl-1H-pyrrol-3-yl)carbamate.

¹H NMR (300 MHz, RT, CDCl₃): δ=1.50 (s, 9H); 1.87 (s, 3H); 7.08 (s, 1H); 7.39-7.51 (m, 3H); 7.53-7.59 (m, 2H); 8.16 (bs, 1H); 8.82 (bs, 1H).

### Intermediate 2D

### tert-Butyl N-(4-benzoyl-1,5-dimethyl-1H-pyrrol-3-yl)carbamate

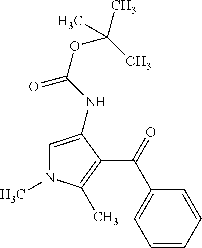 [SMILES: Cc1c(C(=O)c2ccccc2)c(NC(=O)OC(C)(C)C)cn1C]

A solution of 4.4 g of Intermediate 2C, 2.9 ml of dimethyl sulphate and 4.05 g of potassium carbonate in 46 ml of butan-2-one was stirred at 90° C. for 8 h. The mixture was then added to water and extracted three times with dichloromethane and the extracts were washed with water, dried over sodium sulphate and concentrated under reduced pressure. This gave 4.7 g of tert-butyl N-(4-benzoyl-1,5-dimethyl-1H-pyrrol-3-yl)carbamate.

¹H NMR (300 MHz, RT, CDCl₃): δ=1.49 (s, 9H); 1.82 (s, 3H); 3.46 (s, 3H); 7.07 (s, 1H); 7.40-7.46 (m, 2H); 7.47-7.53 (m, 1H); 7.54-7.59 (m, 2H); 8.83 (bs, 1H).

### Intermediate 2E

### 4-Amino-1,2-dimethyl-1H-pyrrol-3-yl phenyl ketone hydrochloride

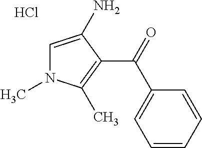 [SMILES: Cl ; Cc1c(C(=O)c2ccccc2)c(N)cn1C]

A solution of 4.2 g of Intermediate 2D in 46.6 ml of HCl in dioxane solution (4 M) was stirred at room temperature for 5 h. The solution was concentrated completely under reduced pressure. This gave 3.4 g of 4-amino-1,2-dimethyl-1H-pyrrol-3-yl phenyl ketone hydrochloride.

¹H NMR (300 MHz, RT, DMSO-d6): δ=1.84 (s, 3H); 3.52 (s, 3H); 7.03 (s, 1H); 7.45-7.54 (m, 2H); 7.55-7.65 (m, 3H); 9.83 (bs, 1H).

### Intermediate 2F

### 2-(3S)-(6,7-Dimethyl-2-oxo-5-phenyl-1,2,3,7-tetrahydropyrrolo[3,4-e][1,4]diazepin-3-yl)acetic acid methyl ester

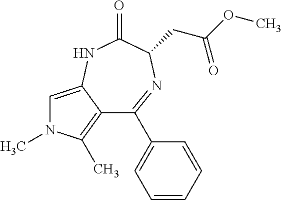

Under argon and at room temperature, a solution of 3.4 g of 2E, 5.01 g of Fmoc-L-Asp(OMe)—OH (CAS 145038-53-5), 4.7 ml triethylamine and 5.16 g of HATU in 52 ml of DMF was stirred for 44 hours. The mixture was partitioned between water and dichloromethane, the organic phase was removed and the aqueous phase was once more extracted with dichloromethane. The combined organic phases were washed with water and saturated sodium chloride solution, dried over sodium sulphate and concentrated. Chromatography on silica gel (hexane/ethyl acetate gradient, then dichloromethane) gave 3.7 g of methyl N-(4-benzoyl-1,5-dimethyl-1H-pyrrol-3-yl)-L-aspartate. These were dissolved in 36 ml of THF, and 0.6 ml of glacial acetic acid was added. The mixture was stirred at room temperature for 5 hours. The mixture was added to water and extracted three times with dichloromethane and the extracts were washed with saturated sodium chloride solution, dried over sodium sulphate and concentrated under reduced pressure. The crude product obtained was purified by chromatography on silica gel (dichloromethane/methanol gradient). This gave 2 g of methyl 2-(35)-(6,7-dimethyl-2-oxo-5-phenyl-1,2,3,7-tetrahydropyrrolo[3,4-e][1,4]diazepin-3-yl)acetate.

¹H NMR (300 MHz, RT, CDCl₃): δ=1.76 (s, 3H); 3.15 (dd, 1H); 3.42 (dd, 1H); 3.53 (s, 3H); 3.73 (s, 3H); 4.43 (t, 1H); 6.44 (s, 1H); 7.31-7.44 (m, 3H); 7.49-7.55 (m, 2H); 7.73 (s, 1H).

### Intermediate 3A

### 2-(3S)-[5-(4-Chlorophenyl)-6,7-dimethyl-2-oxo-1,2,3,7-tetrahydropyrrolo[3,4-e][1,4]diazepin-3-yl]acetic acid tert-butyl ester

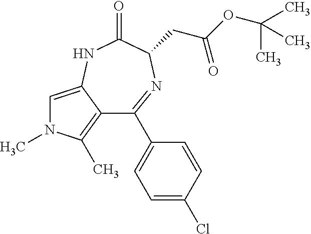 [SMILES: Cc1c2c(cn1C)NC(=O)[C@H](CC(=O)OC(C)(C)C)N=C2c1ccc(Cl)cc1]

Under argon and at room temperature, a solution of 12 g of Intermediate 1E, 17.3 g of Fmoc-L-Asp(0-tert-butyl)-OH (CAS 71989-14-5), 18.3 ml of N,N-diisopropylethylamine and 16 g of HATU in 355 ml of THF was stirred for 14 h. The reaction solution was diluted with ethyl acetate, washed with saturated sodium chloride solution and dried over sodium sulphate, and the solvent was removed under reduced pressure. The residue was taken up in 133 ml of THF, and 11.1 ml of piperidine were added. After stirring at RT for 6 h, 64 ml of glacial acetic acid were added and the mixture was stirred at RT for a further 14 hours. The mixture was added to water and extracted three times with dichloromethane. The combined organic phases were washed with saturated sodium chloride solution and dried over sodium sulphate, and the solvent was removed completely under reduced pressure. The residue was purified by chromatography on silica gel (hexane/ethyl acetate gradient) to obtain 4.55 g of 2-(3S)-[5-(4-chlorophenyl)-6,7-dimethyl-2-oxo-1,2,3,7-tetrahydropyrrolo[3,4-e][1,4]diazepin-3-yl]acetic acid tert-butyl ester.

¹H NMR (300 MHz, RT, CDCl₃): δ=1.47 (s, 9H); 1.79 (s, 3H); 3.04 (dd, 1H); 3.30 (dd, 1H); 3.53 (s, 3H); 4.36 (t, 1H); 6.43 (s, 1H); 7.34 (d, 2H); 7.49 (d, 2H); 7.63 (bs, 1H).

### Intermediate 4A

### 2-(3S)-[6,7-Dimethyl-2-oxo-5-phenyl-1,2,3,7-tetrahydropyrrolo[3,4-e][1,4]diazepin-3-yl]acetic acid tert-butyl ester

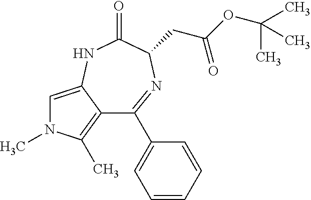 [SMILES: Cc1c2c(cn1C)NC(=O)[C@H](CC(=O)OC(C)(C)C)N=C2c1ccccc1]

Under argon and at room temperature, a solution of 8.4 g of Intermediate 2E, 13.8 g of Fmoc-L-Asp(O-tert-butyl)-OH (CAS 71989-14-5), 8.75 ml of N,N-diisopropylethylamine and 12.74 g of HATU in 250 ml of THF was stirred for 14 h. The reaction solution was diluted with ethyl acetate, washed with saturated sodium chloride solution and dried over sodium sulphate, and the solvent was removed under reduced pressure. The residue was taken up in 200 ml of THF, and 16.6 ml of piperidine were added. After stirring at RT for 2 h, 96 ml of glacial acetic acid were added and the mixture was stirred at RT for a further 14 hours. The mixture was added to water and extracted three times with dichloromethane. The combined organic phases were washed with saturated sodium chloride solution and dried over sodium sulphate, and the solvent was removed completely under reduced pressure. The residue was purified by chromatography on silica gel (hexane/ethyl acetate gradient) to obtain 7.8 g of 2-(3S)-[6,7-dimethyl-2-oxo-5-phenyl-1,2,3,7-tetrahydropyrrolo[3,4-e][1,4]diazepin-3-yl]acetic acid tert-butyl ester.

¹H NMR (400 MHz, RT, DMSO-d6): δ=1.40 (s, 9H); 1.75 (s, 3H); 2.80 (dd, 1H); 3.01 (dd, 1H); 3.55 (s, 3H); 4.09 (t, 1H); 6.61 (s, 1H); 7.38-7.50 (m, 5H); 9.93 (bs, 1H).

### Intermediate 5A

### 2-[(4S)-6-(4-Chlorophenyl)-1,7,8-trim ethyl-4,8-dihydropyrrolo[3,4-f][1,2,4]triazolo [4,3-a][1,4]diazepin-4-yl]acetic acid, sodium salt

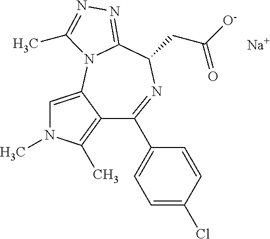 [SMILES: Cc1c2c(cn1C)-n1c(C)nnc1[C@H](CC(=O)[O-])N=C2c1ccc(Cl)cc1 ; [Na+]]

A solution of 550 mg of Example 1, 1.5 ml of aqueous sodium hydroxide solution (1N) in 2.5 ml of methanol was stirred at RT for 14 hours. The solvent was removed completely under reduced pressure, giving 587 mg of 2-[(4S)-6-(4-chlorophenyl)-1,7,8-trimethyl-4,8-dihydropyrrolo[3,4-f][1,2,4]triazolo[4,3-a][1,4]diazepin-4-yl]acetic acid, sodium salt. The substance was used in the next step without further purification.

Rₜ=0.69 min.

UPLC-MS: Instrument: Waters Acquity UPLC-MS SQD; column: Acquity UPLC BEH C18 1.7 50×2.1 mm; eluent A: water+0.1% by vol. of formic acid (99%), eluent B: acetonitrile; gradient: 0-1.6 min 1-99% B, 1.6-2.0 min 99% B; flow rate 0.8 ml/min; T: 60° C.; injection: 2 μl; DAD scan: 210-400 nm

### Intermediate 6A

### 4-Chlorophenyl 1-methyl-4-nitro-1H-pyrazol-3-yl ketone

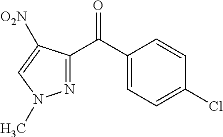 [SMILES: Cn1cc([N+](=O)[O-])c(C(=O)c2ccc(Cl)cc2)n1]

11.45 g of 1-methyl-4-nitro-1H-pyrazole-3-carboxylic acid (CAS 4598-86-1) were added carefully to 52.1 ml of thionyl chloride, and the mixture was then heated at reflux with stirring for 3.5 h. After cooling, the mixture was concentrated under reduced pressure and dried further under oil pump vacuum. This gave 12.75 g of 1-methyl-4-nitro-1H-pyrazole-3-carbonyl chloride which were used in the next step without further purification.

A solution of 12.68 g of the acid chloride prepared beforehand in 200 ml of chlorobenzene was added to a suspension of 8.92 g of aluminium trichloride in 53 ml of chlorobenzene.

Subsequently, the mixture was stirred at 120° C. for 2 h and then at 25° C. for 16 h. The reaction mixture was diluted with 250 ml of ethyl acetate and extracted with 150 ml of water. After phase separation, the aqueous phase was extracted three times with in each case 150 ml of ethyl acetate. The combined organic phases were washed with water and saturated sodium chloride solution, dried over sodium sulphate and concentrated under reduced pressure. The crude product obtained was purified by chromatography on silica gel (hexane/ethyl acetate gradient). This gave 14.7 g of 4-chlorophenyl 1-methyl-4-nitro-1H-pyrazol-3-yl ketone.

¹H NMR (400 MHz, DMSO-d6) δ=3.95 (s, 3H) 7.59-7.64 (m, 2H) 7.85-7.91 (m, 2H) 9.01 (s, 1H).

### Intermediate 6B

### 4-Amino-1-methyl-1H-pyrazol-3-yl 4-chlorophenyl ketone

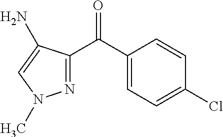 [SMILES: Cn1cc(N)c(C(=O)c2ccc(Cl)cc2)n1]

14.7 g of Intermediate 6A were dissolved in a mixture of 370 ml of ethanol and 185 ml of water, and 30.9 g of iron filings followed by 14.8 g of ammonium chloride were added. Using an air agitator, the orange-brown suspension was stirred at an oil bath temperature of 90° C. for one hour. After cooling, the reaction mixture was filtered through kieselguhr and the filtrate was concentrated under reduced pressure. The residue obtained in this manner was taken up in ethyl acetate and washed with water. After phase separation, the aqueous phase was extracted with ethyl acetate and the combined organic phases were washed once with water and once with saturated sodium chloride solution. After drying over sodium sulphate, the mixture was concentrated under reduced pressure. The crude product obtained in this manner was purified by chromatography on silica gel (hexane/ethyl acetate gradient). This gave 12.6 g of 4-amino-1-methyl-1H-pyrazol-3-yl 4-chlorophenyl ketone.

¹H NMR (400 MHz, DMSO-d6) δ=3.81 (s, 3H) 5.26 (s, 2H) 7.18 (s, 1H) 7.52-7.56 (m, 2H) 8.15-8.20 (m, 2H).

### Intermediate 6C

### N-[3-(4-chlorobenzoyl)-1-methyl-1H-pyrazol-4-yl]-N²-[(9H-fluoren-9-ylmethoxy)carbonyl]-L-aspartic acid methyl ester

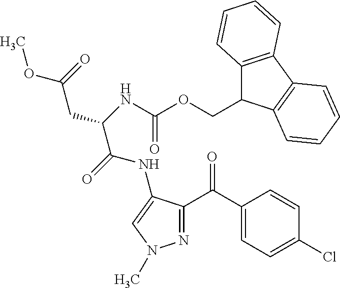 [SMILES: COC(=O)C[C@H](NC(=O)OCC1c2ccccc2-c2ccccc21)C(=O)Nc1cn(C)nc1C(=O)c1ccc(Cl)cc1]

33.4 g of PYBOP and 22.4 ml of N,N-diisopropylethylamine were added to a solution of 11.65 g of Intermediate 6B and 21.9 g of (S)-2-{[(9H-fluoren-9-ylmethoxy)carbonyl]amino}-4-methoxy-4-oxobutanoic acid [Fmoc-L-Asp(OMe)—OH, (CAS 145038-53-5)] in 321 ml of THF. This reaction mixture was stirred at 40° C. for 16 h and, after cooling, concentrated under reduced pressure. The crude product obtained in this manner was combined with an analogous reaction starting with 12.6 g of the title compound from Example 6B which had been stirred at 40° C. for 3 hours and pre-purified by chromatography on silica gel (first hexane/ethyl acetate gradient, then ethyl acetate/methanol gradient with a methanol fraction of up to 25%). The 100 g of crude product obtained in this manner were then purified by chromatography on silica gel (hexane/ethyl acetate gradient). This gave 44.4 g of N-[3-(4-chlorobenzoyl)-1-methyl-1H-pyrazol-4-yl]-N2-[(9H-fluoren-9-ylmethoxy)carbonyl]-L-aspartic acid methyl ester which was pure enough for further reactions.

¹H NMR (400 MHz, DMSO-d6) δ=2.70 (dd, 1H), 2.90 (dd, 1H), 3.58 (s, 3H), 3.95 (s, 3H), 4.22-4.45 (m, 3H), 4.56 (q, 1H), 7.25 (t, 2H), 7.36 (t, 2H), 7.55 (d, 2H), 7.71 (d, 2H), 7.85 (d, 2H), 8.11-8.22 (m, 3H), 8.37 (s, 1H), 10.29 (s, 1H).

### Intermediate 6D

### 2-[(6S)-8-(4-Chlorophenyl)-2-methyl-5-oxo-2,4,5,6-tetrahydropyrazolo[4,3-e][1,4]diazepin-6-yl]acetic acid methyl ester

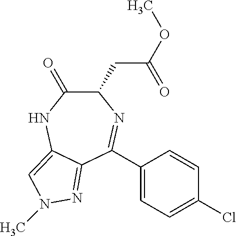 [SMILES: COC(=O)C[C@@H]1N=C(c2ccc(Cl)cc2)c2nn(C)cc2NC1=O]

At 25° C., 5.43 ml of piperidine were quickly added dropwise to a solution of 6.45 g of Intermediate 6C in 84 ml of THF, and the mixture was then stirred at this temperature for one hour. The reaction was checked by UPLC-MS, showing complete conversion to the N-[3-(4-chlorobenzoyl)-1-methyl-1H-pyrazol-4-yl]-L-aspartic acid methyl ester intermediate. 5 ml of acetic acid were then added dropwise to the reaction mixture, and the reaction mixture was stirred at 25° C. for a further three hours. A further 2 ml of acetic acid were added, followed, after a further two hours of stirring, by the addition of another 1 ml of acetic acid. After 16 h of stirring at 25° C., the reaction mixture was then diluted with ethyl acetate and the organic phase was washed with water. After phase separation, the aqueous phase was extracted once with ethyl acetate and the combined organic phases were then washed once with water and once with saturated sodium chloride solution. After drying over sodium sulphate, the mixture was concentrated under reduced pressure. The crude product obtained in this manner was purified by chromatography on silica gel (first hexane/ethyl acetate gradient, then ethyl acetate/methanol gradient with a methanol fraction of up to 50%). This gave 2.87 g of methyl 2-[(6S)-8-(4-chlorophenyl)-2-methyl-5-oxo-2,4,5,6-tetrahydropyrazolo[4,3-e][1,4]diazepin-6-yl]acetate.

¹H NMR (400 MHz, DMSO-d6) δ=2.99 (dd, 1H), 3.18 (dd, 1H), 3.57 (s, 3H), 3.93 (s, 3H), 4.09 (t, 1H), 7.42-7.51 (m, 2H), 7.75 (s, 1H), 7.78-7.85 (m, 2H), 10.43 (s, 1H).

### Intermediate 7A

### 4-Chlorophenyl 1-methyl-4-nitro-1H-pyrazol-5-yl ketone

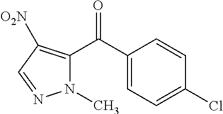 [SMILES: Cn1ncc([N+](=O)[O-])c1C(=O)c1ccc(Cl)cc1]

Analogously to the preparation of Intermediate 6A, 14.4 g of 1-methyl-4-nitro-1H-pyrazole-5-carbonyl chloride (CAS 1006962-20-4) and 320 ml of chlorobenzene gave 2.43 g of 4-chlorophenyl 1-methyl-4-nitro-1H-pyrazol-5-yl ketone as a solid in still contaminated form.

¹H NMR (400 MHz, DMSO-d6) δ=3.80 (s, 3H), 7.62-7.66 (m, 2H), 7.86-7.90 (m, 2H), 8.43 (s, 1H) (characteristic signals of the main component).

### Intermediate 7B

### 4-Amino-1-methyl-1H-pyrazol-5-yl 4-chlorophenyl ketone

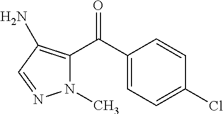 [SMILES: Cn1ncc(N)c1C(=O)c1ccc(Cl)cc1]

Analogously to the preparation of Intermediate 6B, 2.42 g of Intermediate 7A gave 1.11 g of 4-amino-1-methyl-1H-pyrazol-5-yl4-chlorophenyl ketone as a solid in still contaminated form.

¹H NMR (400 MHz, DMSO-d6) δ=3.44 (s, 2H), 4.69 (s, 3H), 7.04 (s, 2H), 7.54-7.59 (m, 2H), 7.63-7.68 (m, 2H) (characteristic signals of the main component).

### Intermediate 7C

### N-[5-(4-chlorobenzoyl)-1-methyl-1H-pyrazol-4-yl]-N²-[(9H-fluoren-9-yl-methoxy)carbonyl]-L-aspartic acid methyl ester

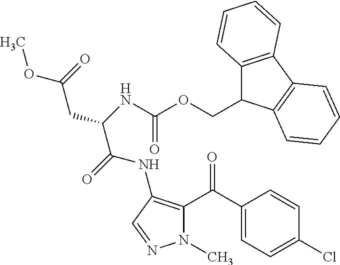 [SMILES: COC(=O)C[C@H](NC(=O)OCC1c2ccccc2-c2ccccc21)C(=O)Nc1cnn(C)c1C(=O)c1ccc(Cl)cc1]

Analogously to the preparation of Intermediate 6C, 1.10 g of Intermediate 7B and 2.08 g of (5)-2-{[(9H-fluoren-9-ylmethoxy)carbonyl]amino}-4-methoxy-4-oxobutanoic acid [Fmoc-L-Asp(OMe)—OH, (CAS 145038-53-5)] gave 3.18 g of methyl N-[5-(4-chlorobenzoyl)-1-methyl-1H-pyrazol-4-yl]-N²-[(9H-fluoren-9-yl-methoxy)carbonyl]-L-aspartate in still contaminated form.

¹H NMR (400 MHz, DMSO-d6): δ [ppm]=2.13-2.20 (m, 2H), 3.52 (s, 3H), 3.81 (s, 3H), 4.11-4.27 (m, 4H), 7.20-7.69 (m, 12H), 7.85 (d, 2H), 9.85 (s, 1H) (characteristic signals of the main component).

### Intermediate 7D

### 2-[(6S)-8-(4-Chlorophenyl)-1-methyl-5-oxo-1,4,5,6-tetrahydropyrazolo[4,3-e][1,4]diazepin-6-yl]acetic acid methyl ester

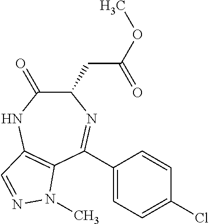 [SMILES: COC(=O)C[C@@H]1N=C(c2ccc(Cl)cc2)c2c(cnn2C)NC1=O]

Analogously to the preparation of Intermediate 6D, 3.18 g of the compound prepared in 7C gave 1.20 g of methyl 2-[(6S)-8-(4-chlorophenyl)-1-methyl-5-oxo-1,4,5,6-tetrahydropyrazolo[4,3-e][1,4]diazepin-6-yl]acetate as a solid in still contaminated form.

¹H NMR (400 MHz, DMSO-d6) δ=3.01 (d, 1H), 3.16-3.27 (m, 1H), 3.39 (s, 3H), 3.58 (s, 3H), 3.94-4.08 (m, 1H), 7.42-7.49 (m, 3H), 7.51-7.56 (m, 2H), 10.66 (s, 1H).

### Intermediate 8A

### 2-(4S)-[6-(4-Chlorophenyl)-1,8-dimethyl-4,8-dihydropyrazolo[3,4-f][1,2,4]triazolo[4,3-a][1,4]diazepin-4-yl]acetic acid, sodium salt

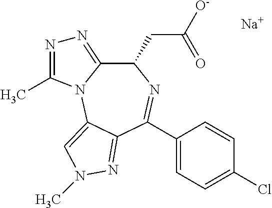 [SMILES: Cc1nnc2n1-c1cn(C)nc1C(c1ccc(Cl)cc1)=N[C@H]2CC(=O)[O-] ; [Na+]]

0.38 ml of a 1N sodium hydroxide solution was added dropwise to a solution of 132 mg of Example 6 in 2.6 ml of methanol. After 1 hour and again after 3 hours of stirring at 25° C., 0.3 ml of water was added. After 4 h of stirring at 25° C. in total, the reaction mixture was concentrated under reduced pressure. After addition of toluene, the mixture was once more concentrated under reduced pressure, and this procedure was repeated a further four times and the product was then dried under oil pump vacuum for 1 hour.

This gave 145 mg of 2-(4S)-[6-(4-chlorophenyl)-1,8-dimethyl-4,8-dihydropyrazolo[3,4-f][1,2,4]triazolo[4,3-a][1,4]diazepin-4-yl]acetic acid, sodium salt. Without further purification, this crude product was used for the amide formation.

### Intermediate 9A

### [(4S)-1,7,8-Trimethyl-6-phenyl-4,8-dihydropyrrolo[3,4-f][1,2,4]triazolo[4,3-a][1,4]diazepin-4-yl]acetic acid, sodium salt

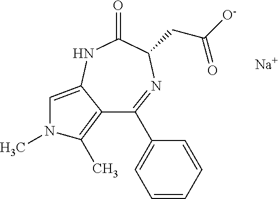 [SMILES: Cc1c2c(cn1C)NC(=O)[C@H](CC(=O)[O-])N=C2c1ccccc1 ; [Na+]]

In analogy to the preparation of Intermediate 5A, 200 mg of Example 4 were used to obtain a crude product of 250 mg of [(4S)-1,7,8-trimethyl-6-phenyl-4,8-dihydropyrrolo[3,4f][1,2,4]triazolo[4,3-a][1,4]diazepin-4-yl]acetic acid, sodium salt. The substance was used in the next step without further purification.

¹H NMR (300 MHz, DMSO-d6) δ=1.78 (s, 3H); 2.54 (s, 3H); 3.24 (dd, 1H); 3.39 (dd, 1H); 3.66 (s, 3H); 4.51 (t, 1H); 7.35-7.54 (m, 6H); 12.35 (bs, 1H).

### PREPARATION OF THE INVENTIVE COMPOUNDS

### EXAMPLES

### Example 1

### 2-(4S)-16-(4-Chlorophenyl)-1,7,8-trimethyl-4,8-dihydropyrrolo[3,4-f][1,2,4]triazolo[4,3-a][1,4]diazepin-4-yl]acetic acid methyl ester

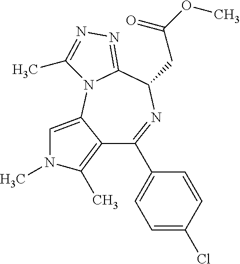

At −78° C. and under argon, 0.9 ml of KOtBu solution (1M in THF) was added to a solution of 300 mg of Intermediate 1F in 2.7 ml of THF. The temperature was increased to −10° C. and stirring was continued for another 30 min. The mixture was cooled again to −78° C. and 173 mg of diethyl chlorophosphate (CAS 814-49-3) were added. Over a period of 30 min, the temperature was increased to −10° C., and stirring was continued for another 2.5 hours. 93 mg of acetylhydrazine were added and the mixture was warmed to RT and stirred for 1 h. After addition of 2.7 ml of butan-1-ol, the mixture was stirred at 85° C. for 4 h. The mixture was concentrated under reduced pressure and purified by chromatography on silica gel (dichloromethane/methanol gradient). This gave 760 mg of a contaminated product which was purified by RP-HPLC (column: C8 Kromasil, mobile phase: methanol/water (0.1% by volume of formic acid) gradient). This gave 42 mg of methyl 2-(4S)-[6-(4-chlorophenyl)-1,7,8-trimethyl-4,8-dihydropyrrolo[3,4-f][1,2,4]triazolo[4,3-a][1,4]diazepin-4-yl]acetate.

¹H NMR (300 MHz, RT, CDCl₃): δ=1.86 (s, 3H); 2.60 (s, 3H); 3.64 (d, 2H); 3.68 (s, 3H); 3.80 (s, 3H); 4.76 (t, 1H); 6.8 (s, 1H); 7.35 (d, 2H); 7.47 (d, 2H).

### Example 2

### 2-(4S)-(1,7,8-Trimethyl-6-phenyl-4,8-dihydropyrrolo[3,4f][1,2,4]triazolo[4,3-a][1,4]diazepin-4-yl)acetic acid methyl ester

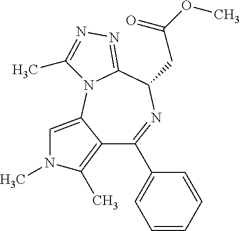

At −78° C. and under argon, 3.3 ml of KOtBu solution (1M in THF) were added to a solution of 1 g of Intermediate 2F in 10 ml of THF. The temperature was increased to −10° C. and stirring was continued for another 30 min. The mixture was cooled again to −78° C. and 637 mg of diethyl chlorophosphate (CAS 814-49-3) were added. Over a period of 30 min, the temperature was increased to −10° C., and stirring was continued for another 2.5 hours. 342 mg of acetylhydrazine were added and the mixture was warmed to RT and stirred for 1 h. After addition of 10 ml of butan-1-ol, the mixture was stirred at 85° C. for 3 h. The mixture was concentrated under reduced pressure and purified by chromatography on silica gel (dichloromethane/methanol gradient). This gave 300 mg of a contaminated product which was purified by RP-HPLC (column: X-Bridge C18 5 μm 100×30 mm, mobile phase: acetonitrile/water (0.1% by volume of formic acid) gradient). This gave 75 mg of methyl 2-(4S)-(1,7,8-trimethyl-6-phenyl-4,8-dihydropyrrolo[3,4-f][1,2,4]triazolo[4,3-a][1,4]diazepin-4-yl)acetate.

¹H NMR (300 MHz, RT, CDCl₃): δ=1.80 (s, 3H); 2.57 (s, 3H); 3.63 (d, 2H); 3.65 (s, 3H); 3.77 (s, 3H); 4.75 (t, 1H); 6.77 (s, 1H); 7.30-7.44 (m, 3H); 7.45-7.51 (m, 2H).

### Example 3

### (−)-2-(4S)-16-(4-Chlorophenyl)-1,7,8-trimethyl-4,8-dihydropyrrolo[3,4-f][1,2,4]triazolo[4,3-a][1,4]diazepin-4-yl]acetic acid tert-butyl ester

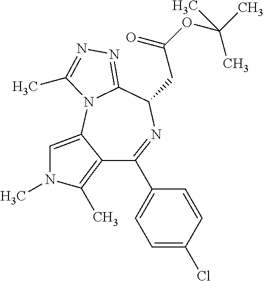 [SMILES: Cc1c2c(cn1C)-n1c(C)nnc1[C@H](CC(=O)OC(C)(C)C)N=C2c1ccc(Cl)cc1]

At −5° C. and under argon, 0.303 g of sodium hydride (60% in oil) were added to a solution of 2 g of Intermediate 3A in 14.2 ml of THF. The mixture was left to warm to RT and stirred for about another 30 min. The mixture was cooled again to −5° C. and 1.81 g of dimorpholinophosphoryl chloride (preparation described in J. Org. Chem. Vol 41, (1976), p. 2720 ff.) were added. Over a period of 30 min, the temperature was increased to 20° C., and stirring was continued for another 1.5 h. 700 mg of acetylhydrazine and 13 ml of butan-1-ol were added, the mixture was stirred for 10 min and the THF was removed completely under reduced pressure. A further 10 ml of butan-1-ol were added, and the mixture was stirred at bath temperature 120° C. for 21 h. The mixture was concentrated under reduced pressure and purified by chromatography on silica gel (dichloromethane/acetone gradient). This gave 1.32 g of 2-(4S)-[6-(4-chlorophenyl)-1,7,8-trimethyl-4,8-dihydropyrrolo[3,4-f][1,2,4]triazolo[4,3-a][1,4]diazepin-4-yl]acetic acid tert-butyl ester.

¹H NMR (300 MHz, RT, CDCl₃): δ=1.50 (s, 9H); 1.83 (s, 3H); 3.51 (d, 2H); 3.66 (s, 3H); 4.71 (t, 1H); 6.86 (s, 1H); 7.34 (d, 2H); 7.46 (d, 2H).

Optical rotation: [α_{D}]=−24.2° (chloroform, c=1 g/100 ml).

### Example 4

### (−)-2-(4S)-(1,7,8-Trimethyl-6-phenyl-4,8-dihydropyrrolo[3,4f][1,2,4]triazolo[4,3-a][1,4]diazepin-4-yl)acetic acid tert-butyl ester

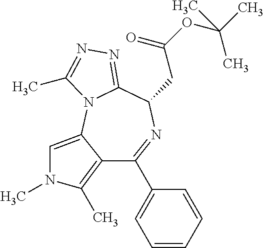 [SMILES: Cc1c2c(cn1C)-n1c(C)nnc1[C@H](CC(=O)OC(C)(C)C)N=C2c1ccccc1]

At −5° C. and under argon, 166 mg of sodium hydride (55-60% in oil) were added to a solution of 1 g of Intermediate 4A in 7.8 ml of THF. The temperature was increased to RT and stirring was continued for another 30 min. The mixture was cooled again to −5° C. and 988 mg of dimorpholinophosphoryl chloride (preparation described in J. Org. Chem. Vol 41, (1976), p. 2720 ff.) were added. The temperature was increased to RT and stirring was continued for another 1.5 hours. 383 mg of acetylhydrazine in 3 ml of butan-1-ol were added. The THF was removed under reduced pressure and another 10 ml of butan-1-ol were added. The mixture was stirred at bath temperature 120° C. for 10 hours. The mixture was concentrated under reduced pressure, taken up with dichloromethane, washed with water and saturated sodium chloride solution, dried with sodium sulphate and concentrated under reduced pressure. The residue was purified by chromatography on silica gel (dichloromethane/methanol gradient). This gave 210 mg of 2-(4S)-1,7,8-trimethyl-6-phenyl-4,8-dihydropyrrolo[3,4-f][1,2,4][triazolo[4,3-a][1,4]diazepin-4-yl]acetic acid tert-butyl ester.

¹H NMR (300 MHz, RT, CDCl₃): δ=1.50 (s, 9H); 1.83 (s, 3H); 3.51 (d, 2H); 3.66 (s, 3H); 4.71 (t, 1H); 6.86 (s, 1H); 7.34 (d, 2H); 7.46 (d, 2H).

Optical rotation: [α_{D}]=−24.2° (chloroform, c=1 g/100 ml).

### Example 5

### (−)-2-[(4S)-6-(4-Chlorophenyl)-1,7,8-trimethyl-4,8-dihydropyrrolo[3,4f][1,2,4]triazolo [4,3-a][1,4]diazepin-4-yl]-1-(3-fluoroazetidin-1-yl)ethan-1-one

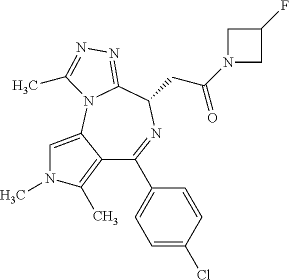 [SMILES: Cc1c2c(cn1C)-n1c(C)nnc1[C@H](CC(=O)N1CC(F)C1)N=C2c1ccc(Cl)cc1]

A solution of 75 mg of Intermediate 5A, 0.1 ml of triethylamine, 105 mg of HATU and 14 mg of 3-fluoroazetidine hydrochloride (CAS 617718-46-4) in 1 ml of DMF was stirred at room temperature overnight. The mixture was added to saturated sodium chloride solution/water and extracted three times with dichloromethane and the extracts were washed twice with saturated sodium chloride solution, dried over sodium sulphate and concentrated under reduced pressure. The crude product obtained was purified by chromatography on silica gel (dichloromethane/methanol gradient). This gave 35 mg of (−)-2-[(4S)-6-(4-chlorophenyl)-1,7,8-trimethyl-4,8-dihydropyrrolo[3,4-f][1,2,4]triazolo[4,3-a][1,4]diazepin-4-yl]-1-(3-fluoroazetidin-1-yl)ethan-1-one.

¹H NMR (300 MHz, RT, CDCl₃): δ=1.81-1.85 (m, 2H); 2.55-2.59 (m, 3H); 3.25-3.45 (m, 2H); 3.65 (s, 3H); 4.10-4.44 (m, 2H); 4.51-4.70 (m, 1H); 4.70-4.98 (m, 2H); 5.38 (bd, 1H); 6.77 (s, 1H); 7.34 (d, 2H); 7.41-7.52 (m, 2H).

Optical rotation: [α_{D}]=−42.4° (methanol, c=1 g/100 ml).

### Example 6

### 2-[(4S)-6-(4-Chlorophenyl)-1,8-dimethyl-4,8-dihydropyrazolo[3,4-f][1,2,4]triazolo[4,3-a][1,4]diazepin-4-yl]acetic acid methyl ester

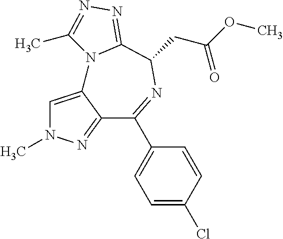

Under argon and at −70° C., 364 mg of NaH (60% suspension in mineral oil) were added carefully to a solution of 2.87 g of Intermediate 6D in 27 ml of THF. The reaction mixture was slowly warmed to 0° C. and, after 20 min, cooled back to −70° C. 1.71 g of diethyl chlorophosphate (CAS 814-49-3) were then added and the mixture was warmed back to 0° C. over a period of 30 min. After a further 30 min of stirring, 1.38 g of acetylhydrazine were added and the reaction mixture was then warmed to 25° C. and stirred for another hour. 27 ml of butanol were then added and the mixture was heated at 85° C. for 2 h. The reaction mixture was added to a little aqueous sodium bicarbonate solution and extracted three times with methylene chloride. The combined organic phases were washed once with saturated sodium chloride solution, dried over sodium sulphate and concentrated under reduced pressure.

The crude product obtained in this manner was purified by chromatography on silica gel (first hexane/ethyl acetate gradient, then ethyl acetate/methanol gradient with a methanol fraction of up to 75%). This gave 277 mg of still contaminated product which was purified in two portions by HPLC chromatography (column: Chromatorex RP C-18 10 μm; 125*30 mm, flow rate 60.00 ml/min, acetonitrile/water/formic acid 15:85:0.1, after 9 minutes acetonitrile/water/formic acid 55:45:0.1 (v/v/v)). The combined product fractions were concentrated under reduced pressure, dissolved in 25 ml of ethyl acetate and washed twice with 15 ml each time of saturated sodium bicarbonate solution. Drying over sodium sulphate and concentration under reduced pressure gave 132 mg of methyl 2-[(4S)-6-(4-chlorophenyl)-1,8-dimethyl-4,8-dihydropyrazolo[3,4-f][1,2,4]triazolo[4,3-a][1,4]diazepin-4-yl]acetate. Analytical HPLC on a chiral carrier material showed that the substance had an ee of 80% (HPLC: Chiralpak IC 3 μm 100×4.6 mm, flow rate 1.0 ml/min, ethanol/methanol/diethylamine 50:50:0.1 (v/v/v).

¹H NMR (400 MHz, DMSO-d6) δ=2.49 (s, 3H), 3.35-3.47 (m, 2H), 3.63 (s, 3H), 4.02 (s, 3H), 4.62 (t, 1H), 7.43-7.48 (m, 2H), 7.67-7.72 (m, 2H), 8.57 (s, 1H).

### Example 7

### 2-[(4S)-6-(4-Chlorophenyl)-1,7-dimethyl-4,7-dihydropyrazolo[3,4-f][1,2,4]triazolo[4,3-a][1,4]diazepin-4-yl]acetic acid methyl ester

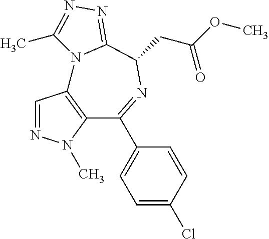

At −70° C., 425 mg of potassium tert-butoxide and, after 30 min of stirring, at −10° C., 714 mg of diethyl chlorophosphate (CAS 814-49-3) were added to a solution of 1.20 g of Intermediate 7D in 11.2 ml of THF. After one hour of stirring at −10° C., 574 mg of acetylhydrazine were added and the mixture was stirred at 25° C. for one hour. This was followed by addition of 11.2 ml of butanol and heating at 110° C. for two hours.

After cooling, the reaction mixture was diluted with dichloromethane and washed in each case once with 10 ml of saturated sodium bicarbonate solution and saturated sodium chloride solution. After drying over sodium sulphate, the mixture was concentrated under reduced pressure. The crude product obtained in this manner was purified by chromatography on silica gel (hexane/ethyl acetate gradient). This gave 150 mg of methyl 2-[(4S)-6-(4-chlorophenyl)-1,7-dimethyl-4,7-dihydropyrazolo[3,4-f][1,2,4]triazolo[4,3-a][1,4]diazepin-4-yl]acetate.

¹H NMR (400 MHz, DMSO-d6) δ=3.37 (dd, 1H), 3.46 (s, 3H), 3.52 (dd, 1H), 3.63 (s, 3H), 4.59 (t, 1H), 7.40-7.46 (m, 2H), 7.50-7.56 (m, 2H), 8.24 (s, 1H).

### Example 8

### (−)-2-[(4S)-6-(4-Chlorophenyl)-1,7,8-trimethyl-4,8-dihydropyrrolo[3,4f][1,2,4]triazolo[4,3-a][1,4]diazepin-4-yl]-1-(1,1-dioxidothiomorpholino)ethan-1-one

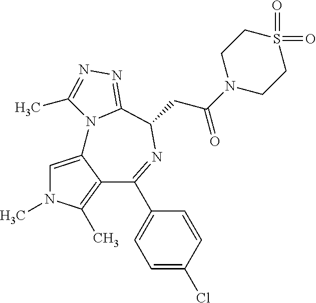 [SMILES: Cc1c2c(cn1C)-n1c(C)nnc1[C@H](CC(=O)N1CCS(=O)(=O)CC1)N=C2c1ccc(Cl)cc1]

A solution of 73 mg of Intermediate 5A, 0.1 ml of triethylamine, 102.5 mg of HATU and 26.7 mg of 1,1-dioxidothiomorpholine (CAS 39093-93-1) in 0.97 ml of DMF was stirred at RT overnight. The mixture was added to saturated sodium chloride solution/water and extracted three times with dichloromethane and the extracts were washed with saturated sodium chloride solution, dried over sodium sulphate and concentrated under reduced pressure. The crude product obtained was purified by chromatography on silica gel (dichloromethane/methanol gradient). This gave 50 mg of (+2-[(4S)-6-(4-chlorophenyl)-1,7,8-trimethyl-4,8-dihydropyrrolo[3,4-f][1,2,4]triazolo[4,3-a][1,4]diazepin-4-yl]-1-(1,1-dioxidothiomorpholin-4yl)ethan-1-one.

¹H NMR (400 MHz, DMSO-d6) δ=1.81 (s, 3H); 3.05 (bq, 2H); 3.33-3.47 (m, 3H); 3.66 (s, 3H); 3.72 (dd, 1H); 3.88 (bq, 2H); 4.10 (bq, 2H); 4.65 (dd, 1H); 7.44 (s, 1H); 7.47 (d, 4H).

Optical rotation: [α_{D}]=−43.2° (methanol, c=1 g/100 ml).

### Example 9

### N-(1-Acetylazetidin-3-yl)-2-(4S)-[6-(4-chlorophenyl)-1,7,8-trimethyl-4,8-dihydropyrrolo[3,4-f][1,2,4]triazolo[4,3-a][1,4]diazepin-4-yl]acetamide

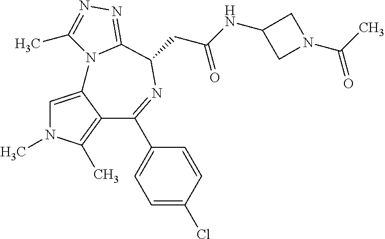 [SMILES: CC(=O)N1CC(NC(=O)C[C@@H]2N=C(c3ccc(Cl)cc3)c3c(cn(C)c3C)-n3c(C)nnc32)C1]

A solution of 73 mg of Intermediate 5A, 0.1 ml of triethylamine, 102.5 mg of HATU and 22.6 mg of 4-aminoazetidinyl methyl ketone (CAS 1137870-15-5) in 0.97 ml of DMF was stirred at RT overnight. The mixture was added to saturated sodium chloride solution/water and extracted three times with dichloromethane and the extracts were washed twice with saturated sodium chloride solution, dried over sodium sulphate and concentrated under reduced pressure. The crude product obtained was purified by RP-HPLC chromatography (XBridge C18 5 μm 100×30 mm, eluent: water/acetonitrile gradient, 0.1% formic acid added, flow rate 50 ml/min) The resulting substance was dissolved in dichloromethane and extracted with sodium bisulphate solution and saturated sodium chloride solution. The solution was dried with sodium sulphate and concentrated under reduced pressure. This gave 2 mg of N-(1-acetylazetidin-3-yl)-2-(4S)-[6-(4-chlorophenyl)-1,7,8-trimethyl-4,8-dihydropyrrolo[3,4-f][1,2,4]triazolo[4,3-a][1,4]diazepin-4-yl]acetamide.

¹H NMR (300 MHz, DMSO-d6) δ=1.77 (d, 3H); 1.81 (s, 3H); 3.20 (d, 2H); 3.66 (s, 3H); 3.67-3.76 (m, 1H); 3.88-3.98 (m, 1H); 4.01-4.14 (m, 1H); 4.36 (dd, 1H); 4.40-4.49 (m, 1H); 4.56 (t, 1H); 7.43 (s, 1H); 7.45 (d, 4H); 8.84 (bs, 1H).

### Example 10

### (−)-2-[(4S)-6-(4-Chlorophenyl)-1,7,8-trimethyl-4,8-dihydropyrrolo[3,4f][1,2,4]triazolo[4,3-a][1,4]diazepin-4-yl]-N-ethylacetamide

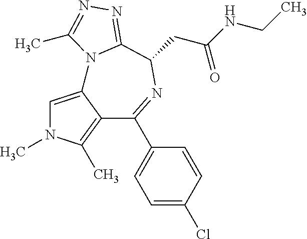 [SMILES: CCNC(=O)C[C@@H]1N=C(c2ccc(Cl)cc2)c2c(cn(C)c2C)-n2c(C)nnc21]

A solution of 250 mg of Intermediate 5A, 0.363 ml of triethylamine, 372 mg of HATU and 32.3 mg of ethylamine in 3.5 ml of DMF was stirred at RT overnight. The mixture was added to saturated sodium chloride solution/water and extracted three times with dichloromethane and the extracts were washed with saturated sodium chloride solution, dried over sodium sulphate and concentrated under reduced pressure. The crude product obtained was purified by chromatography on silica gel (dichloromethane/methanol gradient). This gave 50 mg of (−)-2-[(4S)-6-(4-chlorophenyl)-1,7,8-trimethyl-4,8-dihydropyrrolo[3,4-f][1,2,4]triazolo[4,3-a][1,4]diazepin-4-yl]-N-ethylacetamide.

¹H NMR (300 MHz, DMSO-d6) δ=1.06 (t, 3H); 1.81 (s, 3H); 2.48 (s, 3H); 3.02-3.32 (m, 4H); 3.66 (s, 3H); 4.57 (dd, 1H); 7.43 (s, 1H); 7.46 (s, 4H); 8.16 (dt, 1H).

Optical rotation: [α_{D}]=−55.4° (methanol, c=1 g/100 ml).

### Example 11

### (−)-2-[(4S)-6-(4-Chlorophenyl)-1,7,8-trimethyl-4,8-dihydropyrrolo[3,4f][1,2,4]triazolo[4,3-a][1,4]diazepin-4-yl]-N-methyl-N-[(3-methyloxetan-3-yl)methyl]acetamide

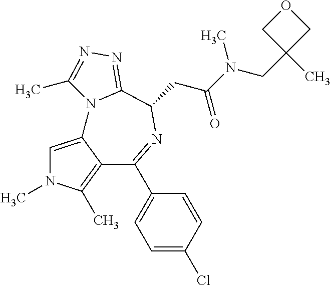

A solution of 70 mg of Intermediate 5A, 0.1 ml of triethylamine, 98.5 mg of HATU and 20 mg of (methyl)[(3-methyloxetan-3-yl)methyl]amine (CAS 915919-90-3) in 1 ml of DMF was stirred at room temperature overnight. The mixture was added to saturated sodium chloride solution/water and extracted three times with dichloromethane and the extracts were washed with saturated sodium chloride solution, dried over sodium sulphate and concentrated under reduced pressure. The crude product obtained was purified by chromatography on silica gel (dichloromethane/methanol gradient). This gave 35 mg of (+2-[(4S)-6-(4-chlorophenyl)-1,7,8-trimethyl-4,8-dihydropyrrolo[3,4-f][1,2,4]triazolo[4,3-a][1,4]diazepin-4-yl]-N-methyl-N-[(3-methyloxetan-3-yl)methyl]acetamide.

¹H NMR (400 MHz, CDCl₃) δ=1.37 (s, 3H); 1.82 (s, 3H); 2.56 (s, 3H); 3.26 (s, 3H); 3.55-3.72 (m+s, 7H); 4.32 (dd, 2H); 4.63-4.71 (m, 2H); 4.89 (t, 1H); 6.76 (s, 1H); 7.32 (d, 2H); 7.45 (d, 2H).

Optical rotation: [α_{D}]=−88.1° (methanol, c=1 g/100 ml).

### Example 12

### (−)-1-{[(4S)-6-(4-Chlorophenyl)-1,7,8-trimethyl-4,8-dihydropyrrolo[3,4-f][1,2,4]triazolo[4,3-a][[1,4]diazepin-4-yl]acetyl}pyrrolidin-3-one

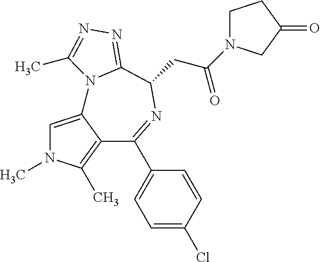 [SMILES: Cc1c2c(cn1C)-n1c(C)nnc1[C@H](CC(=O)N1CCC(=O)C1)N=C2c1ccc(Cl)cc1]

A solution of 73 mg of Intermediate 5A, 0.1 ml of triethylamine, 103 mg of HATU and 30 mg of pyrrolid-3-one hydrochloride in 1 ml of DMF was stirred at room temperature overnight. The mixture was added to saturated sodium chloride solution/water and extracted three times with dichloromethane and the extracts were washed with saturated sodium chloride solution, dried over sodium sulphate and concentrated under reduced pressure. The crude product obtained was purified by chromatography on silica gel (dichloromethane/methanol gradient). This gave 44 mg of (−)-1-{[(4S)-6-(4-chlorophenyl)-1,7,8-trimethyl-4,8-dihydropyrrolo[3,4-f][1,2,4]triazolo[4,3-a][1,4]diazepin-4-yl]acetyl}pyrrolidin-3-one.

¹H NMR (400 MHz, DMSO-d6, selected signals) δ=1.81 (s, 3H); 2.61 (t, 1H); 2.72 (t, 1H); 3.24 (dd, 1H); 3.48 (dd, 1H); 3.57 (dd, 1H); 3.66 (s, 3H); 3.69-3.77 (m, 2H); 4.07-4.15 (m, 1H); 4.21 (d, 1H); 4.65 (q, 1H); 7.44 (s, 1H); 7.45-7.52 (m, 4H).

Optical rotation: [α_{D}]=−45.3° (methanol, c=1 g/100 ml).

### Example 13

### 2-[(4S)-6-(4-Chlorophenyl)-1,7,8-trimethyl-4,8-dihydropyrrolo[3,4-f][1,2,4]triazolo[4,3-a][1,4]diazepin-4-yl]-N-(2-oxo-2,3-dihydro-1H-indol-5-yl)acetamide

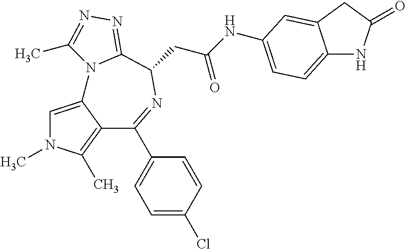 [SMILES: Cc1c2c(cn1C)-n1c(C)nnc1[C@H](CC(=O)Nc1ccc3c(c1)CC(=O)N3)N=C2c1ccc(Cl)cc1]

In analogy to the preparation of Example 12, 73 mg of Intermediate 5A and 29 mg of 5-amino-2-oxo-2,3-dihydro-1H-indole (CAS 20876-36-2) were used to obtain 92 mg of 2-[(4S)-6-(4-chlorophenyl)-1,7,8-trimethyl-4,8-dihydropyrrolo[3,4-f][1,2,4]triazolo[4,3-a][1,4]diazepin-4-yl]-N-(2-oxo-2,3-dihydro-1H-indol-5-yl)acetamide.

¹H NMR (400 MHz, DMSO-d6, selected signals) δ=1.81 (s, 3H); 3.46 (s, 2H); 3.67 (s, 3H); 3.65 (t, 1H); 6.75 (d, 1H); 7.38 (d, 1H); 7.42-7.51 (m, 6H); 7.56 (s, 1H); 10.12 (s, 1H); 10.27 (s, 1H).

Optical rotation: [α_{D}]=−18.3° (methanol, c=1 g/100 ml).

### Example 14

### 2-[6-(4-Chlorophenyl)-1,8-dimethyl-4,8-dihydropyrazolo[3,4-f][1,2,4]triazolo[4,3-a][1,4]diazepin-4-yl]-1-(1,1-dioxidothiomorpholin-4-yl)ethanone

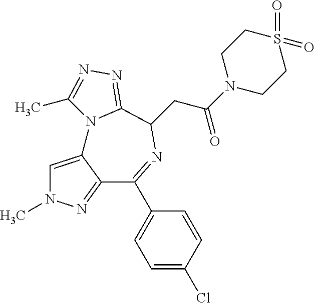 [SMILES: Cc1nnc2n1-c1cn(C)nc1C(c1ccc(Cl)cc1)=NC2CC(=O)N1CCS(=O)(=O)CC1]

In analogy to the preparation of Example 12, 323 mg of Intermediate 8A and 111 mg of thiomorpholine 1,1-dioxide (CAS 39093-93-1) were used to obtain 140 mg of 2-[6-(4-chlorophenyl)-1,8-dimethyl-4,8-dihydropyrazolo[3,4-f][1,2,4]triazolo[4,3-a][1,4]diazepin-4-yl]-1-(1,1-dioxidothiomorpholin-4-yl)ethanone.

¹H NMR (400 MHz, DMSO-d6) δ=2.52 (s, 3H); 2.98-3.13 (m, 2H); 3.38-3.46 (m, 1H); 3.50 (dd, 1H); 3.74 (dd, 1H); 3.80-3.98 (m, 2H); 4.02-4.20 (m+s, 5H); 4.73 (t, 1H); 7.49 (d, 2H); 7.75 (d, 2H); 8.60 (s, 1H).

### Example 15

### 2-[(4S)-6-(4-Chlorophenyl)-1,8-dimethyl-4,8-dihydropyrazolo[3,4f][1,2,4]triazolo[4,3-a][1,4]diazepin-4-yl]-1-(1,1-dioxidothiomorpholin-4-yl)ethanone

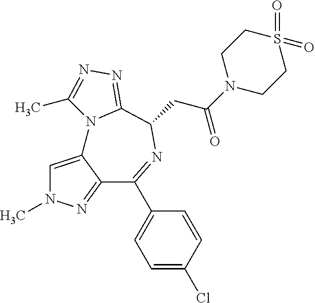 [SMILES: Cc1nnc2n1-c1cn(C)nc1C(c1ccc(Cl)cc1)=N[C@H]2CC(=O)N1CCS(=O)(=O)CC1]

124 mg of 2-[6-(4-Chlorophenyl)-1,8-dimethyl-4,8-dihydropyrazolo[3,4-f][1,2,4]triazolo[4,3-a][1,4]diazepin-4-yl]-1-(1,1-dioxidothiomorpholin-4-yl)ethanone were separated into the enantiomers by chiral HPLC (Chiralpak IA 5 μm 250×20 mm, CO₂/ethanol (0.5% diethylamine) 60:40 (v/v), flow rate: 80 ml/min, 150 bar, 40° C.).

Yield: 50 mg of 2-[(4S)-6-(4-chlorophenyl)-1,8-dimethyl-4,8-dihydropyrazolo[3,4-f][1,2,4]triazolo[4,3-a][1,4]diazepin-4-yl]-1-(1,1-dioxidothiomorpholin-4-yl)ethanone.

¹H NMR (400 MHz, DMSO-d6) δ=2.52 (s, 3H); 2.98-3.13 (m, 2H); 3.38-3.46 (m, 1H); 3.50 (dd, 1H); 3.74 (dd, 1H); 3.80-3.98 (m, 2H); 4.02-4.20 (m+s, 5H); 4.73 (t, 1H); 7.49 (d, 2H); 7.75 (d, 2H); 8.60 (s, 1H).

### Example 16

### 2-[6-(4-Chlorophenyl)-1,8-dimethyl-4,8-dihydropyrazolo[3,4-f][1,2,4]triazolo[4,3-a][1,4]diazepin-4-yl]-1-(morpholin-4-yl)ethanone

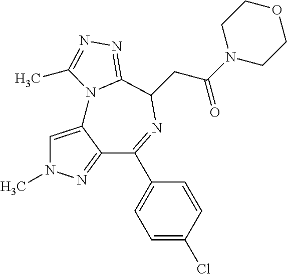 [SMILES: Cc1nnc2n1-c1cn(C)nc1C(c1ccc(Cl)cc1)=NC2CC(=O)N1CCOCC1]

In analogy to the preparation of Example 12, 290 mg of Intermediate 8A and 64 mg of morpholine were used to obtain 92 mg of 2-[6-(4-chlorophenyl)-1,8-dimethyl-4,8-dihydropyrazolo[3,4-f][1,2,4]triazolo[4,3-a][1,4]diazepin-4-yl]-1-(morpholin-4-yl)ethanone.

¹H NMR (400 MHz, DMSO-d6) δ=2.51 (s, 3H); 3.42 (dd, 1H); 3.43-3.49 (m, 2H); 3.52-3.58 (m, 2H), 3.59 (dd, 1H); 3.66 (s, 4H); 4.05 (s, 3H); 4.73 (t, 1H); 7.49 (d, 2H); 7.74 (d, 2H); 8.61 (s, 1H).

### Example 17

### 2-[6-(4-Chlorophenyl)-1,8-dimethyl-4,8-dihydropyrazolo[3,4-f][1,2,4]triazolo[4,3-a][1,4]diazepin-4-yl]-1-(3-fluoroazetidin-1-yl)ethanone

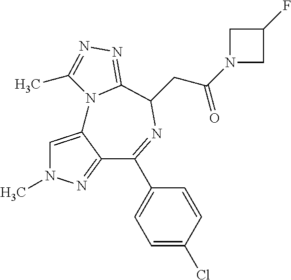 [SMILES: Cc1nnc2n1-c1cn(C)nc1C(c1ccc(Cl)cc1)=NC2CC(=O)N1CC(F)C1]

In analogy to the preparation of Example 12, 323 mg of Intermediate 8A and 92 mg of 3-fluoroazetidine hydrochloride (CAS 617718-46-4) were used to obtain 4.7 mg of 2-[6-(4-chlorophenyl)-1,8-dimethyl-4,8-dihydropyrazolo[3,4-f][1,2,4]triazolo[4,3-a][1,4]diazepin-4-yl]-1-(3-fluoroazetidin-1-yl)ethanone.

¹H NMR (400 MHz, DMSO-d6) δ=3.13-3.30 (m, 2H); 3.82-4.00 (m, 1H); 4.05 (s, 3H); 4.11-4.31 (m, 1H); 4.35-4.55 (m, 1H); 4.63 (t, 1H); 4.58-4.80 (m, 1H); 4.47 (bd, 1H); 7.49 (dd, 2H); 7.74 (dd, 2H); 8.59 (s, 1H).

### Example 18

### 2-[6-(4-Chlorophenol)-1,8-dimethyl-4,8-dihydropyrazolo[3,4-f][1,2,4]triazolo[4,3-a][1,4]diazepin-4-yl]-N-ethylacetamide

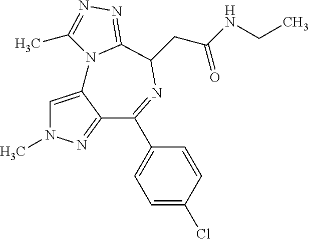 [SMILES: CCNC(=O)CC1N=C(c2ccc(Cl)cc2)c2nn(C)cc2-n2c(C)nnc21]

In analogy to the preparation of Example 12, 290 mg of Intermediate 8A and 0.37 ml of ethylamine (2 M in THF) were used to obtain 29 mg of 2-[6-(4-chlorophenyl)-1,8-dimethyl-4,8-dihydropyrazolo[3,4-f][1,2,4]triazolo[4,3-a][1,4]diazepin-4-yl]-N-ethylacetamide.

¹H NMR (400 MHz, DMSO-d6) δ=1.06 (t, 3H); 3.03-3.01 (m. 3H); 3.25 (dd, 1H); 4.05 (s, 3H); 4.65 (dd, 1H); 7.49 (d, 2H); 7.73 (d, 2H); 8.18 (t, 1H); 8.58 (s, 1H).

### Example 19

### 1-(Morpholin-4-yl)-2-[(4S)-1,7,8-trimethyl-6-phenyl-4,8-dihydropyrrolo[3,4f][1,2,4]triazolo[4,3-a][1,4]diazepin-4-yl]ethanone

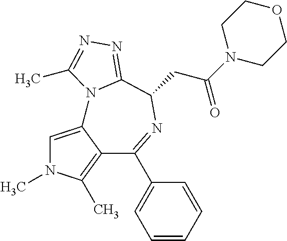 [SMILES: Cc1c2c(cn1C)-n1c(C)nnc1[C@H](CC(=O)N1CCOCC1)N=C2c1ccccc1]

In analogy to the preparation of Example 12, 125 mg of Intermediate 9A and 34 mg of morpholine were used to obtain 10 mg of 1-(morpholin-4-yl)-2-[(4S)-1,7,8-trimethyl-6-phenyl-4,8-dihydropyrrolo[3,4-f][1,2,4]triazolo[4,3-a][1,4]diazepin-4-yl]ethanone.

¹H NMR (400 MHz, DMSO-d6) δ=1.77 (s, 3H); 2.60 (s, 3H); 3.53 (dd, 1H); 3.54-3.61 (m, 4H); 3.61-3.71 (m, 5H); 3.75 (s, 3H); 5.18 (dd, 1H); 7.61-7.70 (m, 5H); 7.76-7.83 (m, 1H).

### Example 20

### (−)-2-[(4S)-6-(4-Chlorophenyl)-1,7,8-trimethyl-4,8-dihydropyrrolo[3,4f][1,2,4]triazolo[4,3-a][1,4]diazepin-4-yl]-1-(3-phenoxyazetidin-1-yl)ethanone

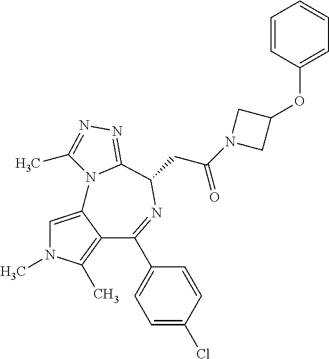 [SMILES: Cc1c2c(cn1C)-n1c(C)nnc1[C@H](CC(=O)N1CC(Oc3ccccc3)C1)N=C2c1ccc(Cl)cc1]

In analogy to the preparation of Example 12, 200 mg of Intermediate 5A and 85 mg of 3-phenoxyazetidine hydrochloride (CAS 301335-39-7) were used to obtain 46 mg of (−)-2-[(4S)-6-(4-chlorophenyl)-1,7,8-trimethyl-4,8-dihydropyrrolo[3,4-f][1,2,4]triazolo[4,3-a][1,4]diazepin-4-yl]-1-(3-phenoxyazetidin-1-yl)ethanone.

¹H NMR (400 MHz, DMSO-d6) δ=1.81 (d, 3H); 3.05-3.30 (m, 2H); 3.66 (s, 3H); 3.83 (ddd, 1H); 4.30-4.40 (m, 2H); 4.51-4.58 (m, 1H); 4.82 (ddd, 1H); 5.05-5.15 (m, 1H); 6.89 (dd, 2H); 7.00 (t, 1H); 7.33 (dt, 2H); 7.38-7.51 (m, 5H).

Optical rotation: [α_{D}]=−33.6° (methanol, c=1 g/100 ml).

### Example 21

### (−)-2-[(4S)-6-(4-Chlorophenyl)-1,7,8-trimethyl-4,8-dihydropyrrolo[3,4-f][[1,2,4]triazolo[4,3-a][1,4]diazepin-4-yl]-1-(morpholin-4-yl) ethanone

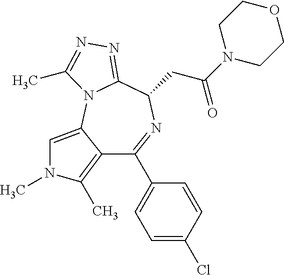 [SMILES: Cc1c2c(cn1C)-n1c(C)nnc1[C@H](CC(=O)N1CCOCC1)N=C2c1ccc(Cl)cc1]

In analogy to the preparation of Example 12, 500 mg of Intermediate 5A and 70 mg of morpholine were used to obtain 185 mg of (−)-2-[(4S)-6-(4-chlorophenyl)-1,7,8-trimethyl-4,8-dihydropyrrolo[3,4-f][1,2,4]triazolo[4,3-a][1,4]diazepin-4-yl]-1-(morpholin-4-yl)ethanone.

¹H NMR (400 MHz, DMSO-d6) δ=1.81 (s, 3H); 3.41-3.73 (m+s, 13H); 4.67 (t, 1H); 7.46 (s, 1H); 7.48 (s, 4H).

Optical rotation: [α_{D}]=−11.0° (methanol, c=1 g/100 ml).

### Example 22

### (−)-4-{[(4S)-6-(4-Chlorophenyl)-1,7,8-trimethyl-4,8-dihydropyrrolo[3,4-f][1,2,4]triazolo[4,3-a][1,4]diazepin-4-yl]acetyl}piperazine-1-carboxylic acid tert-butyl ester

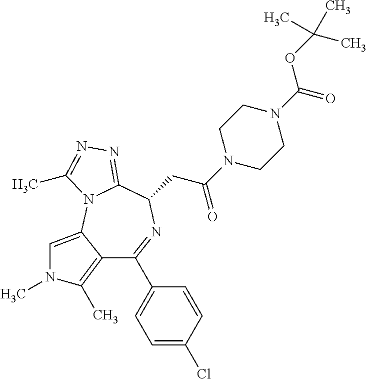 [SMILES: Cc1c2c(cn1C)-n1c(C)nnc1[C@H](CC(=O)N1CCN(C(=O)OC(C)(C)C)CC1)N=C2c1ccc(Cl)cc1]

In analogy to the preparation of Example 12, 500 mg of Intermediate 5A and 150 mg of piperazine-1-carboxylic acid tert-butyl ester (CAS 57260-71-6) were used to obtain 225 mg of (−)-4-{[(4S)-6-(4-chlorophenyl)-1,7,8-trimethyl-4,8-dihydropyrrolo[3,4-f][1,2,4]triazolo[4,3-a][1,4]diazepin-4-yl]acetyl}piperazine-1-carboxylic acid tert-butyl ester.

¹H NMR (400 MHz, DMSO-d6, selected signals) δ=1.43 (s, 9H); 1.81 (s, 3H); 3.41-3.50 (m, 4H); 3.60 (dd, 1H); 3.66 (s, 3H); 4.66 (t, 1H); 7.43 (s, 1H); 7.46 (s, 4H).

Optical rotation: [α_{D}]=−48.7° (methanol, c=1 g/100 ml).

### Example 23

### (−)-2-{[(4S)-6-(4-Chlorophenyl)-1,7,8-trim ethyl-4,8-dihydropyrrolo[3,4-f][1,2,4]triazolo[4,3-a][1,4]diazepin-4-yl]acetyl}hexahydropyrrolo[1,2-a]pyrazin-6(2H)-one

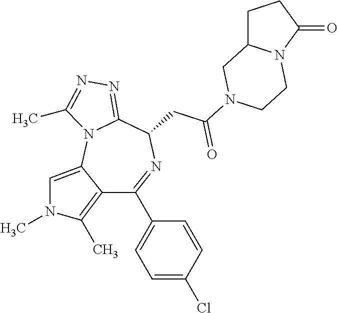 [SMILES: Cc1c2c(cn1C)-n1c(C)nnc1[C@H](CC(=O)N1CCN3C(=O)CCC3C1)N=C2c1ccc(Cl)cc1]

In analogy to the preparation of Example 12, 500 mg of Intermediate 5A and 201 mg of hexahydropyrrolo[1,2-a]pyrazin-6(2H)-one (CAS 117810-52-3) were used to obtain 70 mg of (−)-2-{[(4S)-6-(4-chlorophenyl)-1,7,8-trimethyl-4,8-dihydropyrrolo[3,4-f][1,2,4]triazolo[4,3-a][1,4]diazepin-4-yl]acetyl}hexahydropyrrolo[1,2-a]pyrazin-6(2H)-one.

¹H NMR (400 MHz, DMSO-d6, selected signals) δ=1.50-1.68 (m, 1H); 1.81 (s, 3H); 2.01-2.32 (m, 2H); 2.80-3.12 (m, 2H); 3.57-3.75 (m+s, 5H); 3.85 (dd, 1H); 4.16-4.57 (m, 2H); 4.66 (t, 1H); 7.45 (s, 1H); 7.47 (s, 4H).

Optical rotation: [α_{D}]=−64.7° (methanol, c=1 g/100 ml).

### Example 24

### 6-(4-Chlorophenyl)-4-[(3-cyclopropyl-1,2,4-oxadiazol-5-yl)methyl]-1,7,8-trimethyl-4,8-dihydropyrrolo[3,4-f][1,2,4]triazolo[4,3-a][1,4]diazepine

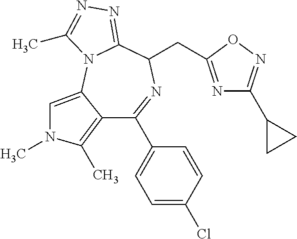 [SMILES: Cc1c2c(cn1C)-n1c(C)nnc1C(Cc1nc(C3CC3)no1)N=C2c1ccc(Cl)cc1]

1 g of N′-hydroxycyclopropanecarboximidamide (CAS 51285-13-3) in 6 ml of 1-methyl-2-pyrrolidone was stirred at RT until a homogeneous solution formed. To this were added 880 mg of (−)-2-[6-(4-chlorophenyl)-1,7,8-trimethyl-4,8-dihydropyrrolo[3,4-f][1,2,4]triazolo[4,3-a][1,4]diazepin-4-yl]acetic acid tert-butyl ester (Example 3) and, gradually, 432 mg of sodium methoxide. The mixture was stirred at 70° C. for 3 h. The mixture was added to water and extracted three times with ethyl acetate. The combined organic phases were washed with water. The solid that appeared was filtered off with suction. 300 mg of 6-(4-chlorophenyl)-4-[(3-cyclopropyl-1,2,4-oxadiazol-5-yl)methyl]-1,7,8-trimethyl-4,8-dihydropyrrolo[3,4-f][1,2,4]triazolo[4,3-a][1,4]diazepine were obtained.

¹H NMR (300 MHz, CDCl₃) δ=1.05 (d, 4H); 1.85 (s, 3H); 2.10 (p, 1H); 2.58 (s, 3H); 3.67 (s, 3H); 4.00 (dd, 1H); 4.19 (dd, 1H); 4.76 (dd, 1H); 6.80 (s, 1H); 7.31 (d, 2H); 7.38 (d, 2H).

### Example 25

### 6-(4-Chlorophenyl)-1,7,8-trimethyl-4-[(3-methyl-1,2,4-oxadiazol-5-yl)methyl]-4,8-dihydropyrrolo[3,4-f][1,2,4]triazolo[4,3-a][1,4]diazepine

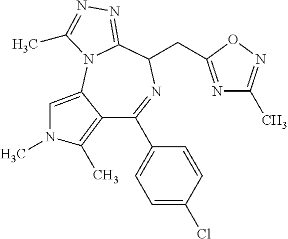

In analogy to the preparation of Example 24, 880 mg of Example 3 and 741 mg of acetimide oxime (CAS 22059-22-9) were used to obtain 280 mg of 6-(4-chlorophenyl)-1,7,8-trimethyl-4-[(3-methyl-1,2,4-oxadiazol-5-yl)methyl]-4,8-dihydropyrrolo[3,4-f][1,2,4]triazolo[4,3-a][1,4]diazepine.

¹H NMR (300 MHz, CDCl₃) δ=1.85 (s, 3H); 2.41 (s, 3H); 2.59 (s, 3H); 3.67 (s, 3H); 4.08 (dd, 1H); 4.20 (dd, 1H); 4.82 (dd, 1H); 6.80 (s, 1H); 7.32 (d, 2H); 7.39 (d, 2H).

### Example 26

### (4S)-6-(4-Chlorophenyl)-1,7,8-trimethyl-4-[(3-methyl-1,2,4-oxadiazol-5-yl)methyl]-4,8-dihydropyrrolo[3,4-f][1,2,4]triazolo[4,3-a][1,4]diazepine

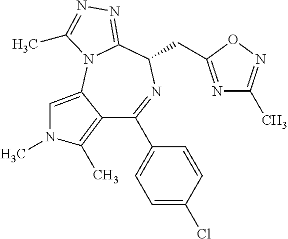

140 mg of 6-(4-chlorophenyl)-1,7,8-trimethyl-4-[(3-methyl-1,2,4-oxadiazol-5-yl)methyl]-4,8-dihydropyrrolo[3,4-f][1,2,4]triazolo[4,3-a][1,4]diazepine (Example 25) were separated into the enantiomers by HPLC on a chiral stationary phase (Chiralpak ID 5 μm 250×20 mm, CO₂/methanol 70:30 (v/v), flow rate: 80 ml/min, 150 bar, 40° C.).

Yield: 71 mg of (4S)-6-(4-chlorophenyl)-1,7,8-trimethyl-4-[(3-methyl-1,2,4-oxadiazol-5-yl)methyl]-4,8-dihydropyrrolo[3,4-f][1,2,4]triazolo[4,3-a][1,4]diazepine.

¹H NMR (300 MHz, CDCl₃) δ=1.05 (d, 4H); 1.85 (s, 3H); 2.10 (p, 1H); 2.58 (s, 3H); 3.67 (s, 3H); 4.00 (dd, 1H); 4.19 (dd, 1H); 4.76 (dd, 1H); 6.80 (s, 1H); 7.31 (d, 2H); 7.38 (d, 2H).

### Example 27

### 6-(4-Chlorophenyl)-1,7,8-trimethyl-4-[(3-ethyl-1,2,4-oxadiazol-5-yl)methyl]-4,8-dihydropyrrolo[3,4-f][1,2,4]triazolo[4,3-a][1,4]diazepine

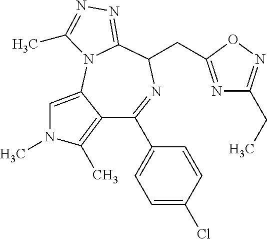 [SMILES: CCc1noc(CC2N=C(c3ccc(Cl)cc3)c3c(cn(C)c3C)-n3c(C)nnc32)n1]

In analogy to the preparation of Example 24, 880 mg of Example 3 and 881 mg of N-hydroxypropionamidine (CAS 29335-36-2) were used to obtain 450 mg of 6-(4-chlorophenyl)-1,7,8-trimethyl-4-[(3-ethyl-1,2,4-oxadiazol-5-yl)methyl]-4,8-dihydropyrrolo[3,4-f][1,2,4]triazolo[4,3-a][1,4]diazepine.

¹H NMR (300 MHz, CDCl₃) δ=1.35 (t, 3H); 1.85 (s, 3H); 2.59 (s, 3H); 2.78 (q, 2H); 3.67 (s, 3H); 4.06 (dd, 1H); 4.23 (dd, 1H); 4.82 (dd, 1H); 6.80 (s, 1H); 7.32 (d, 2H); 7.38 (d, 2H).

### Example 28

### (4S)-6-(4-Chlorophenyl)-1,7,8-trimethyl-4-[(3-ethyl-1,2,4-oxadiazol-5-yl)methyl]-4,8-dihydropyrrolo[3,4-f][1,2,4]triazolo[4,3-a][1,4]diazepin e

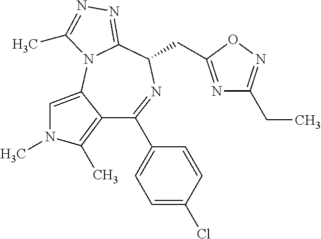 [SMILES: CCc1noc(C[C@@H]2N=C(c3ccc(Cl)cc3)c3c(cn(C)c3C)-n3c(C)nnc32)n1]

440 mg of 6-(4-chlorophenyl)-1,7,8-trimethyl-4-[(3-ethyl-1,2,4-oxadiazol-5-yl)methyl]-4,8-dihydropyrrolo[3,4-f][1,2,4]triazolo[4,3-a][1,4]diazepine (Example 27) were separated into the enantiomers by chiral HPLC (Chiralpak IA 5 μm 250×30 mm, hexane/methanol/diethylamine 70:30:0.1 (v/v/v), flow rate: 50 ml/min, RT).

Yield: 174 mg of (4S)-6-(4-chlorophenyl)-1,7,8-trimethyl-4-[(3-ethyl-1,2,4-oxadiazol-5-yl)methyl]-4,8-dihydropyrrolo[3,4-f][1,2,4]triazolo[4,3-a][1,4]diazepine.

¹H NMR (300 MHz, CDCl₃) δ=1.35 (t, 3H); 1.85 (s, 3H); 2.59 (s, 3H); 2.78 (q, 2H); 3.67 (s, 3H); 4.06 (dd, 1H); 4.23 (dd, 1H); 4.82 (dd, 1H); 6.80 (s, 1H); 7.32 (d, 2H); 7.38 (d, 2H).

### Example 29

### 6-(4-Chlorophenyl)-1,7,8-trimethyl-4-{[3-(propan-2-yl)-1,2,4-oxadiazol-5-yl]methyl}-4,8-dihydropyrrolo[3,4-f][1,2,4]triazolo[4,3-a][1,4]diazepine

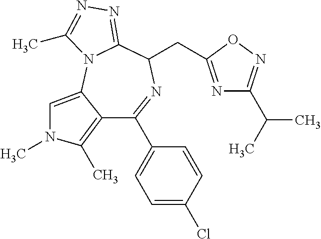 [SMILES: Cc1c2c(cn1C)-n1c(C)nnc1C(Cc1nc(C(C)C)no1)N=C2c1ccc(Cl)cc1]

In analogy to the preparation of Example 24, 880 mg of Example 3 and 1.02 g of N-hydroxyisobutyramidine (CAS 35613-84-4) were used to obtain 420 mg of 6-(4-chlorophenyl)-1,7,8-trimethyl-4-{[3-(propan-2-yl)-1,2,4-oxadiazol-5-yl]methyl}-4,8-dihydropyrrolo[3,4-f][1,2,4]triazolo[4,3-a][1,4]diazepine.

¹H NMR (300 MHz, CDCl₃) δ=1.37 (d, 6H); 1.85 (s, 3H); 2.59 (s, 3H); 3.17 (sep, 1H); 3.68 (s, 3H); 4.03 (dd, 1H); 4.25 (dd, 1H); 4.81 (dd, 1H); 6.80 (s, 1H); 7.31 (d, 2H); 7.37 (d, 2H).

### Example 30

### (4S)-6-(4-Chlorophenyl)-1,7,8-trimethyl-4-{[3-(propan-2-yl)-1,2,4-oxadiazol-5-yl]methyl}-4,8-dihydropyrrolo[3,4-f][1,2,4]triazolo[4,3-a][1,4]diazepine

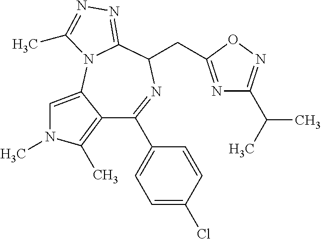 [SMILES: Cc1c2c(cn1C)-n1c(C)nnc1C(Cc1nc(C(C)C)no1)N=C2c1ccc(Cl)cc1]

410 mg of 6-(4-chlorophenyl)-1,7,8-trimethyl-4-{[3-(propan-2-yl)-1,2,4-oxadiazol-5-yl]methyl}-4,8-dihydropyrrolo[3,4-f][1,2,4]triazolo[4,3-a][1,4]diazepine (Example 29) were separated into the enantiomers by chiral HPLC (Chiralpak ID 5 μm 250×20 mm, hexane/ethanol/diethylamine 70:30:0.1 (v/v/v), flow rate: 30 ml/min, RT).

Yield: 170 mg of (4S)-6-(4-chlorophenyl)-1,7,8-trimethyl-4-{[3-(propan-2-yl)-1,2,4-oxadiazol-5-yl]methyl}-4,8-dihydropyrrolo[3,4-f][1,2,4]triazolo[4,3-a][1,4]diazepine.

1H NMR (300 MHz, CDCl₃) δ=1.37 (d, 6H); 1.85 (s, 3H); 2.59 (s, 3H); 3.17 (sep, 1H); 3.68 (s, 3H); 4.03 (dd, 1H); 4.25 (dd, 1H); 4.81 (dd, 1H); 6.80 (s, 1H); 7.31 (d, 2H); 7.37 (d, 2H).

### Example 31

### 4-[(3-tert-Butyl-1,2,4-oxadiazol-5-yl)methyl]-6-(4-chlorophenyl)-1,7,8-trimethyl-4,8-dihydropyrrolo[3,4-f][1,2,4]triazolo[4,3-a][1,4]diazepine

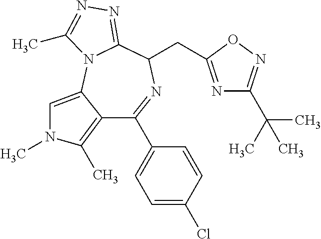 [SMILES: Cc1c2c(cn1C)-n1c(C)nnc1C(Cc1nc(C(C)(C)C)no1)N=C2c1ccc(Cl)cc1]

In analogy to the preparation of Example 24, 440 mg of Example 3 and 581 mg of N-hydroxy-2,2-dimethylpropanimidamide (CAS 42956-75-2) were used to obtain 450 mg of 4-[(3-tert-butyl-1,2,4-oxadiazol-5-yl)methyl]-6-(4-chlorophenyl)-1,7,8-trimethyl-4,8-dihydropyrrolo[3,4-f][1,2,4]triazolo[4,3-a][1,4]diazepine.

¹H NMR (300 MHz, CDCl₃) δ=1.40 (s, 9H); 1.84 (s, 3H); 2.58 (s, 3H); 3.67 (s, 3H); 4.00 (dd, 1H); 4.26 (dd, 1H); 4.79 (dd, 1H); 6.81 (s, 1H); 7.29 (d, 2H); 7.36 (d, 2H).

### Biological Efficacy of the Inventive Compounds

### 1. Assays

### 1.1 Protein-Protein Interaction Assay

### Binding Assay BRD4/Acetylated Peptide H4 (“PRO”)

To assess the BRD4 binding strength of the substances described in this application, the ability thereof to inhibit the interaction between BRD4 (BD1) and acetylated histone H4 in a dose-dependent manner was quantified.

For this purpose, a time-resolved fluorescence resonance energy transfer (TR-FRET) assay was used, which measures the binding between N-terminally His₆-tagged BRD4 (BD1) (amino acids 67-152, longer constructs also being possible, preferably amino acids 44-168) and a synthetic acetylated histone H4 (Ac-H4) peptide with sequence GRGK(Ac)GGK(Ac)GLGK(Ac)GGAK(Ac)RHGSGSK-biotin. The recombinant BRD4 protein produced in-house according to Filippakopoulos et al., Nature, 2010, 468:1119-1123 was expressed in E. coli and purified by means of (Ni-NTA) affinity and (Sephadex G-75) size exclusion chromatography. The Ac-H4 peptide can be purchased, for example, from Biosyntan (Berlin, Germany).

In the assay, typically 11 different concentrations of each substance (0.1 nM, 0.33 nM, 1.1 nM, 3.8 nM, 13 nM, 44 nM, 0.15 μM, 0.51 μM, 1.7 μM, 5.9 μM and 20 μM) were analysed as duplicates on the same microtitre plate. For this purpose, 100-fold concentrated solutions in DMSO were prepared by serial dilutions (1:3.4) of a 2 mM stock solution into a clear, 384-well microtitre plate (Greiner Bio-One, Frickenhausen, Germany). From this, 50 nl were transferred into a black test plate (Greiner Bio-One, Frickenhausen, Germany). The test was started by the addition of 2 μl of a 2.5-fold concentrated BRD4 solution (final concentration typically 10 nM in the 5 μl of reaction volume) in aqueous assay buffer [50 mM HEPES pH 7.5, 50 mM sodium chloride (NaCl), 0.25 mM CHAPS and 0.05% serum albumin (BSA)] to the substances in the test plate. This was followed by a 10-minute incubation step at 22° C. for the pre-equilibration of putative complexes between BRD4 and the substances. Subsequently, 3 μl of a 1.67-fold concentrated solution (in assay buffer) consisting of Ac-H4 peptide (83.5 nM) and TR-FRET detection reagents [16.7 nM anti-6His-XL665 and 3.34 nM streptavidin cryptate (both from Cisbio Bioassays, Codolet, France), and 668 mM potassium fluoride (KF)] were added.

The mixture was then incubated in the dark at 22° C. for one hour and then at 4° C. for at least 3 hours and for no longer than overnight. The formation of BRD4/Ac-H4 complexes was determined by the measurement of the resonance energy transfer from the streptavidin-Eu cryptate to the anti-6His-XL665 antibody present in the reaction. For this purpose, the fluorescence emission was measured at 620 nm and 665 nm after excitation at 330-350 nm in a TR-FRET measuring instrument, for example a Rubystar or Pherastar (both from BMG Lab Technologies, Offenburg, Germany) or a Viewlux (Perkin-Elmer). The ratio of the emissions at 665 nm and at 622 nm was taken as an indicator of the amount of BRD4/Ac-H4 complexes formed.

The data (ratios) obtained were normalized, with 0% inhibition corresponding to the mean from the measurements for a set of controls (typically 32 data points) in which all the reagents were present. In these, in place of test substances, 50 nl of DMSO (100%) were used Inhibition of 100% corresponded to the mean from the measurements for a set of controls (typically 32 data points) in which all the reagents except BRD4 were present. The IC₅₀ was determined by regression analysis based on a 4-parameter equation (minimum, maximum, IC₅₀, Hill; Y=max+(min−max)/(1+(X/IC₅₀)^{Hill})).

### 1.2 Cell Assays

In accordance with the invention, the ability of the substances to inhibit cell proliferation was determined Cell viability was determined by means of the alamarBlue® reagent (Invitrogen) in a Victor X3 Multilabel Reader (Perkin Elmer). The excitation wavelength was 530 nm and the emission wavelength 590 nM.

The MOLM-13 cells (DSMZ, ACC 554) were sown at a concentration of 4000 cells/well in 100 μl of growth medium (RPMI1640, 10% FCS) on 96-well microtitre plates.

The MV4-11 cells (ATCC, CRL 9591) were sown at a concentration of 5000 cells/well in 100 μl of growth medium (RPMI1640, 10% FCS) on 96-well microtitre plates.

The B16F10 cells (ATCC, CRL-6475) were sown at a concentration of 300-500 cells/well in 100 μl of growth medium (DMEM with phenol red, 10% FCS) on 96-well microtitre plates.

The LOX-IMVI cells (NCI-60) were sown at a concentration of 1000 cells/well in 100 μl of growth medium (RPMI1640, 10% FCS) on 96-well microtitre plates.

The MOLP-8 cells (DSMZ, ACC 569) were sown at a concentration of 4000 cells/well in 100 μl of growth medium (RPMI1640, 20% FCS) on 96-well microtitre plates.

The KMS-12-PE cells (DSMZ, ACC 606) were sown at a concentration of 4000 cells/well in 100 μl of growth medium (RPMI1640, 20% FCS) on 96-well microtitre plates.

The LAPC-4 cells (ATCC, PTA-1441TM) were sown at a concentration of 4000 cells/well in 100 μl of growth medium (RPMI1640, 2 mM L-glutamine, 10% cFCS) on 96-well microtitre plates. One day later, the LAPC-4 cells were treated with 1 nM methyltrienolone and various substance dilutions.

The MDA-MB-231 cells (DSMZ, ACC 732) were sown at a concentration of 4000 cells/well in 100 μl of growth medium (DMEM/Ham's F12 medium, 10% FCS) on 96-well microtitre plates. After overnight incubation at 37° C., the fluorescence values (CI values) were determined. Then the plates were treated with various substance dilutions (1E-5 M, 3E-6 M, 1E-6 M, 3E-7 M, 1E-7 M, 3E-8 M, 1E-8 M) and incubated at 37° C. for 72 (MV-4-11, LOX-IMVI cells), 96 (MOLM-13, B16F10, MDA-MB-431 cells), 120 (MOLP-8, KMS-12-PE cells) or 168 (LAPC-4 cells) hours. Subsequently, the fluorescence values were determined (CO values). For the data analysis, the CI values were subtracted from the CO values and the results were compared between cells which had been treated with various dilutions of the substance or only with buffer solution. The ICso values (substance concentration needed for 50% inhibition of cell proliferation) were calculated therefrom.

The substances were tested in the cell lines in Table 1, which represent the indications specified by way of example:

TABLE 1

| Cell line | Source | Indication |
|---|---|---|
| MOLM-13 | DSMZ | acute myeloid leukaemia |
| MV4-11 | ATCC | acute myeloid leukaemia |
| B16F10 | ATCC | melanoma (BRAF wild-type) |
| LOX IMVI | NCI-60 | melanoma (BRAF mutated) |
| MOLP-8 | DSMZ | multiple myeloma |
| KMS-12-PE | DSMZ | multiple myeloma |
| LAPC-4 | ATCC | prostate cancer |
| MDA-MB-231 | DSMZ | mammary carcinoma |

### 2. Results

### 2.1 Binding Assay

Table 2 shows the results from the BRD4 (BD1) binding assay.

TABLE 2

| | IC₅₀ (BRD4) |
|---|---|
| Example | (μmol/l) |
| 1 | 0.01 |
| 2 | 0.01 |
| 3 | 0.03 |
| 4 | 0.31 |
| 5 | 0.07 |
| 6 | 0.14 |
| 7 | 0.23 |
| 8 | 0.1 |
| 9 | 0.11 |
| 10 | 0.17 |
| 11 | 0.22 |
| 12 | 0.11 |
| 13 | 0.04 |
| 14 | 0.64 |
| 15 | 0.45 |
| 16 | 0.70 |
| 17 | 1.14 |
| 18 | 0.78 |
| 19 | 0.24 |
| 20 | 0.06 |
| 21 | 0.04 |
| 22 | 0.02 |
| 23 | 0.05 |
| 24 | 0.05 |
| 25 | 0.09 |
| 26 | 0.02 |
| 27 | 0.03 |
| 28 | 0.03 |
| 29 | 0.03 |
| 30 | 0.03 |
| 31 | 0.49 |

### 2.2 Cell Assays

Tables 3A and 3B show the results of various cell proliferation assays.

TABLE 3A

| | Leukaemia | | Melanoma | | Multiple myeloma | |
|---|---|---|---|---|---|---|
| | MOLM- | | | LOX | | KMS- |
| | 13 | MV4-11 | B16F10 | IMVI | MOLP-8 | 12-PE |
| Exam- | IC₅₀ | IC₅₀ | IC₅₀ | IC₅₀ | IC₅₀ | IC₅₀ |
| ple | (μmol/l) | (μmol/l) | (μmol/l) | (μmol/l) | (μmol/l) | (μmol/l) |
| 1 | 0.13 | | 0.08 | | 0.05 | |
| 2 | 0.33 | | 0.08 | | | |
| 3 | 0.21 | | 0.19 | | 0.19 | |
| 5 | 0.53 | | 0.58 | | 0.37 | |
| 6 | 0.45 | | 0.55 | | 0.45 | |
| 7 | 0.74 | | | | 0.45 | |
| 8 | 0.56 | | 0.67 | | 0.44 | |
| 9 | 0.61 | | | | | |
| 10 | 0.45 | | | | | |
| 11 | 1.99 | | 1.40 | | | |
| 12 | 0.48 | | 0.60 | | | |
| 13 | 0.19 | | 0.33 | | 0.22 | |
| 14 | 3.46 | | | | 4.00 | |
| 15 | 2.98 | | | | | |
| 16 | 4.55 | | | | | |
| 19 | 1.24 | | | | | |
| 20 | 0.92 | | | | | |
| 21 | 0.52 | | | | | |
| 22 | 0.72 | | | | | |
| 23 | 0.88 | | | | | |
| 24 | 0.08 | | | | | |
| 25 | 0.13 | | | | | |
| 26 | 0.08 | | | | 0.05 | |
| 27 | 0.10 | | | | | |
| 28 | 0.07 | | | | 0.04 | |
| 29 | 0.08 | | | | | |
| 30 | 0.07 | | | | 0.05 | |
| 31 | 0.49 | | | | | |

TABLE 3B

| | Prostate | Breast |
|---|---|---|
| | LAPC-4 | MDA-MB-231 |
| Example | IC₅₀ (μmol/l) | IC₅₀ (μmol/l) |
| 1 | 0.05 | 0.09 |
| 2 | 0.03 | 0.09 |
| 3 | 0.10 | |
| 5 | 0.37 | |
| 6 | 0.25 | |
| 8 | 0.36 | |
| 9 | 0.55 | |
| 11 | 0.81 | |
| 12 | 0.22 | |
| 13 | 0.09 | |
| 19 | 0.70 | |
| 20 | 0.41 | |
| 21 | 0.24 | |
| 22 | 0.34 | |
| 23 | 0.47 | |
| 25 | 0.08 | |
| 31 | 0.35 | |

### 3. Determination of Stability in Human Plasma

Isolated human liver microsomes (HLM) were used to assess the metabolic stability of compounds of general formula I.

The incubations were conducted with 2.4 ml of HLM solution (protein content 0.5 mg/ml), 30 μl of the test compound (final concentration 1 μM) and 0.6 ml of the cofactor mixture (=NADPH-generating system composed of 3 IU glucose-6-phosphate dehydrogenase, 14.6 mg glucose-6-phosphate, 1.2 mg NADP) at 37° C. in 100 mM phosphate buffer at pH 7.4. Samples were taken at 6 time points (2-60 min) and precipitated with an equal volume of methanol, and the recovery of the test substances used in the supernatant was determined by LC-MS/MS analysis. The half-life of substance degradation determined therefrom was used to calculate what is called the intrinsic clearance of the substance in the liver microsome preparation. With the aid of this, using various physiological parameters, a (metabolic) in vivo clearance with respect to phase I reactions was predicted according to the well-stirred model.

1. A compound of formula I 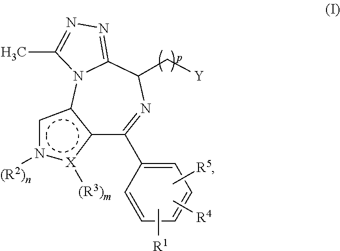
in which
X is a carbon or nitrogen atom,
n and m are each independently 0 or 1,
p is 1, 2, 3 or 4,
R¹, R⁴ and R⁵ are each independently hydrogen, hydroxyl, cyano, nitro, amino, aminocarbonyl, halogen or C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl, wherein the C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl is optionally substituted singly or multiply, identically or differently by halogen, amino, hydroxyl, carboxyl, hydroxy-C₁-C₆-alkyl, C₁-C₆-alkoxy, alkoxy-C₁-C₆-alkyl, C₁-C₆-alkylamino or amino-C₁-C₆-alkyl,
R² is hydrogen or is C₁-C₆-alkyl, aminocarbonyl, C₁-C₆-alkylcarbonyl, C₁-C₆-alkylaminocarbonyl, C₁-C₆-alkylsulphonyl, phenylsulphonyl or C₁-C₆-alkylaminosulphonyl, each optionally mono- or polysubstituted identically or differently by halogen, amino, hydroxyl, carboxyl, hydroxy-C₁-C₆-alkyl, C₁-C₆-alkoxy, C₁-C₆-alkoxy-C₁-C₆-alkyl, C₁-C₆-alkylamino, amino-C₁-C₆-alkyl, monocyclic heterocyclyl having 4 to 8 ring atoms or monocyclic heteroaryl having 5 or 6 ring atoms, in which the monocyclic heterocyclyl and heteroaryl are optionally monosubstituted by C₁-C₃-alkyl,
or
is C₃-C₁₀-cycloalkyl optionally mono- or polysubstituted identically or differently by halogen, amino, hydroxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy or a monocyclic heterocyclyl radical having 4 to 8 ring atoms,
or
is monocyclic heteroaryl which has 5 or 6 ring atoms and is optionally mono- or polysubstituted identically or differently by halogen, amino, hydroxyl, cyano, nitro, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₁-C₆-alkylcarbonyl, C₁-C₆-alkylsulphonyl, C₃-C₁₀-cycloalkyl or a monocyclic heterocyclyl radical having 4 to 8 ring atoms,
or
is monocyclic heterocyclyl which has 4 to 8 ring atoms and is optionally mono- or polysubstituted identically or differently by halogen, amino, hydroxyl, cyano, oxo, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylamino-C₁-C₆-alkyl, hydroxy-C₁-C₆-alkyl, halo-C₁-C₆-alkyl, halo-C₁-C₆-alkoxy, C₁-C₆-alkylcarbonyl, C₁-C₆-alkylsulphonyl, C₃-C₁₀-cycloalkyl or a monocyclic heterocyclyl radical having 4 to 8 ring atoms,
or
is phenyl optionally mono- or polysubstituted identically or differently by halogen, amino, hydroxyl, cyano, nitro, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylaminocarbonyl, C₁-C₆-alkylaminosulphonyl, C₁-C₆-alkylamino-C₁-C₆-alkyl, hydroxy-C₁-C₆-alkyl, halo-C₁-C₆-alkyl, halo-C₁-C₆-alkoxy, C₁-C₆-alkylcarbonyl, C₁-C₆-alkylsulphonyl, C₃-C₁₀-cycloalkyl or a monocyclic heterocyclyl radical having 4 to 8 ring atoms,
R³ is hydrogen, hydroxyl, cyano, nitro, amino, aminocarbonyl, halogen or is C₁-C₆-alkyl, C₁-C₆-alkoxy, C₁-C₆-alkylcarbonyl, C₁-C₆-alkylsulphonyl, phenylsulphonyl, C₁-C₆-alkylamino, C₁-C₆-alkylcarbonylamino, C₁-C₆-alkylaminocarbonyl or C₁-C₆-alkylaminosulphonyl, each optionally mono- or polysubstituted identically or differently by halogen, amino, hydroxyl, carboxyl, hydroxy-C₁-C₆-alkyl, C₁-C₆-alkoxy, C₁-C₆-alkoxy-C₁-C₆-alkyl, C₁-C₆-alkylamino or amino-C₁-C₆-alkyl, when X is a carbon atom,
or
R³ is hydrogen or is C₁-C₆-alkyl, C₁-C₆-alkoxy, C₁-C₆-alkylcarbonyl, C₁-C₆-alkylaminocarbonyl, C₁-C₆-alkylsulphonyl, phenylsulphonyl or C₁-C₆-alkylaminosulphonyl, each optionally mono- or polysubstituted identically or differently by halogen, amino, hydroxyl, carboxyl, hydroxy-C₁-C₆-alkyl, C₁-C₆-alkoxy, C₁-C₆-alkoxy-C₁-C₆-alkyl, C₁-C₆-alkylamino or amino-C₁-C₆-alkyl, when X is a nitrogen atom,
or
R² and R³ together with the ring atoms N and X may form a further heteroaromatic or heterocyclic ring having 5 to 7 ring atoms which may optionally be mono- or polysubstituted identically or differently by halogen, amino, hydroxyl, cyano, nitro, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylaminocarbonyl, C₁-C₆-alkylaminosulphonyl, C₁-C₆-alkylcarbonyl, C₁-C₆-alkylsulphonyl, C₁-C₆-alkylamino-C₁-C₆-alkyl, hydroxy-C₁-C₆-alkyl, halo-C₁-C₆-alkyl, halo-C₁-C₆-alkoxy, C₃-C₁₀-cycloalkyl or a monocyclic heterocyclyl radical having 4 to 8 ring atoms,
Y is a —C(═O)OR¹², —C(═O)R¹³ or —C(═O)NR¹⁰R¹¹ group, or is phenyl, monocyclic heterocyclyl having 4 to 8 ring atoms or mono- or bicyclic heteroaryl having 5 to 10 ring atoms, in which phenyl, heteroaryl and heterocyclyl are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, phenoxy, halophenyl, phenyl-C₁-C₆-alkyl, phenyl-C₁-C₆-alkoxy, heteroaryl having 5 or 6 ring atoms, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ or a monocyclic heterocyclyl radical which has 4 to 8 ring atoms and is itself optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl or halo-C₁-C₆-alkoxy,
R⁶ and R⁷ are each independently hydrogen, C₁-C₃-alkyl, C₃-C₇-cycloalkyl or di-C₁-C₃-alkylamino-C₁-C₃-alkyl, each optionally mono- or polysubstituted identically or differently by halogen, cyano, amino or oxo,
R⁸ is hydroxyl, C₁-C₆-alkyl, C₁-C₆-alkoxy, halo-C₁-C₃-alkyl, hydroxy-C₁-C₃-alkyl, C₁-C₃-alkoxy-C₁-C₃-alkyl, C₃-C₈-cycloalkyl, phenyl, monocyclic heterocyclyl having 4 to 8 ring atoms or monocyclic heteroaryl having 5 or 6 ring atoms, in which phenyl, heteroaryl and heterocyclyl are optionally mono- or disubstituted by halogen, C₁-C₃-alkoxy or C₁-C₃-alkyl,
R⁹ is C₁-C₆-alkyl or C₃-C₈-cycloalkyl,
R¹⁰ and R¹¹ are each independently hydrogen, C₁-C₆-alkyl or a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, C₁-C₆-alkylcarbonylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, phenoxy, monocyclic heteroaryl having 5 or 6 ring atoms, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ or a monocyclic heterocyclyl radical having 4 to 8 ring atoms,
or
R¹⁰ and R¹¹ together with the adjacent nitrogen atom are a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, C₁-C₆-alkylcarbonylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, phenoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ or a monocyclic heterocyclyl radical having 4 to 8 ring atoms,
R¹² is C₁-C₆-alkyl, halo-C₁-C₃-alkyl, hydroxy-C₁-C₃-alkyl, C₁-C₃-alkoxy-C₁-C₃-alkyl, C₃-C₈-cycloalkyl, phenyl, a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, C₁-C₆-alkylcarbonylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, phenoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ or a monocyclic heterocyclyl radical having 4 to 8 ring atoms,
R¹³ is C₁-C₆-alkyl, halo-C₁-C₃-alkyl, hydroxy-C₁-C₃-alkyl, C₁-C₃-alkoxy-C₁-C₃-alkyl, C₃-C₈-cycloalkyl, phenyl, a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, C₁-C₆-alkylcarbonylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, phenoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ or a monocyclic heterocyclyl radical having 4 to 8 ring atoms,
or an enantiomer, diastereomer, tautomer, solvate, physiologically acceptable salt or solvate of a salt thereof.

2. A compound according to claim 1, in which
X is a carbon or nitrogen atom,
n and m are each independently 0 or 1,
p is 1,
R¹, R⁴ and R⁵ are each independently hydrogen, hydroxyl, cyano, nitro, amino, aminocarbonyl, halogen or C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl, wherein the C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl is optionally substituted singly or multiply, identically or differently by halogen, amino, hydroxyl, carboxyl, hydroxy-C₁-C₆-alkyl, C₁-C₆-alkoxy, C₁-C₆-alkoxy-C₁-C₆-alkyl, C₁-C₆-alkylamino or amino-C₁-C₆-alkyl,
R² is hydrogen or is C₁-C₆-alkyl, C₁-C₆-alkylcarbonyl, phenylsulphonyl or C₁-C₆-alkylsulphonyl, each optionally mono- or polysubstituted identically or differently by halogen, amino, hydroxyl, carboxyl, hydroxy-C₁-C₆-alkyl, C₁-C₆-alkoxy, C₁-C₆-alkoxy-C₁-C₆-alkyl, C₁-C₆-alkylamino, amino-C₁-C₆-alkyl, monocyclic heterocyclyl having 4 to 8 ring atoms or monocyclic heteroaryl having 5 or 6 ring atoms, in which the monocyclic heterocyclyl and heteroaryl are optionally monosubstituted by C₁-C₃-alkyl,
or
is C₃-C₁₀-cycloalkyl optionally mono- or polysubstituted identically or differently by halogen, amino, hydroxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy or a monocyclic heterocyclyl radical having 4 to 8 ring atoms,
or
is monocyclic heteroaryl which has 5 or 6 ring atoms and is optionally mono- or polysubstituted identically or differently by halogen, amino, hydroxyl, cyano, nitro, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₁-C₆-alkylcarbonyl, C₁-C₆-alkylsulphonyl, C₃-C₁₀-cycloalkyl or a monocyclic heterocyclyl radical having 4 to 8 ring atoms,
or
is monocyclic heterocyclyl which has 4 to 8 ring atoms and is optionally mono- or polysubstituted identically or differently by halogen, amino, hydroxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₁-C₆-alkylsulphonyl or a monocyclic heterocyclyl radical having 4 to 8 ring atoms,
or
is phenyl optionally mono- or polysubstituted identically or differently by halogen, amino, hydroxyl, cyano, nitro, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylaminocarbonyl, C₁-C₆-alkylaminosulphonyl, C₁-C₆-alkylamino-C₁-C₆-alkyl, hydroxy-C₁-C₆-alkyl, halo-C₁-C₆-alkyl, halo-C₁-C₆-alkoxy, C₁-C₆-alkylsulphonyl, C₃-C₁₀-cycloalkyl or a monocyclic heterocyclyl radical having 4 to 8 ring atoms,
R³ is hydrogen or is C₁-C₆-alkyl, C₁-C₆-alkylcarbonyl, C₁-C₆-alkylsulphonyl or phenylsulphonyl, each optionally mono- or polysubstituted identically or differently by halogen, amino, hydroxyl, carboxyl, hydroxy-C₁-C₆-alkyl, C₁-C₆-alkoxy, C₁-C₆-alkoxy-C₁-C₆-alkyl, C₁-C₆-alkylamino or amino-C₁-C₆-alkyl, when X is a carbon atom,
or
R³ is hydrogen or is C₁-C₆-alkyl, C₁-C₆-alkylcarbonyl, C₁-C₆-alkylsulphonyl or phenylsulphonyl, each optionally mono- or polysubstituted identically or differently by halogen, amino, hydroxyl, carboxyl, hydroxy-C₁-C₆-alkyl, C₁-C₆-alkoxy, C₁-C₆-alkoxy-C₁-C₆-alkyl, C₁-C₆-alkylamino or amino-C₁-C₆-alkyl, when X is a nitrogen atom,
or
R² and R³ together with the ring atoms N and X may form a further heteroaromatic or heterocyclic ring which has 5 to 7 ring atoms and is optionally mono- or polysubstituted identically or differently by halogen, amino, hydroxyl, cyano, nitro, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylaminocarbonyl, C₁-C₆-alkylaminosulphonyl, C₁-C₆-alkylcarbonyl, C₁-C₆-alkylsulphonyl, C₁-C₆-alkylamino-C₁-C₆-alkyl, hydroxy-C₁-C₆-alkyl, halo-C₁-C₆-alkyl, halo-C₁-C₆-alkoxy, C₃-C₁₀-cycloalkyl or a monocyclic heterocyclyl radical having 4 to 8 ring atoms,
Y is a —C(═O)OR¹², —C(═O)R¹³ or —C(═O)NR¹⁰R¹¹ group, or is phenyl, monocyclic heterocyclyl having 4 to 8 ring atoms or mono- or bicyclic heteroaryl having 5 to 10 ring atoms, in which phenyl, heteroaryl and heterocyclyl are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, phenoxy, halophenyl, phenyl-C₁-C₆-alkyl, phenyl-C₁-C₆-alkoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, or a monocyclic heterocyclyl radical which has 4 to 8 ring atoms and is itself optionally mono- or polysubstituted identically or differently by halogen, cyano, nitro, hydroxyl, amino, oxo, carboxyl, C₁-C₆-alkyl, C₁-C₆-alkoxy, hydroxy-C₁-C₆-alkyl, C₁-C₆ alkylamino, amino C₁-C₆ alkyl C₁-C₆ alkylamino C₁-C₆ alkyl halo-C₁-C₆-alkyl or halo-C₁-C₆-alkoxy,
R⁶ and R⁷ are each independently hydrogen, C₁-C₃-alkyl, cyclopropyl or di-C₁-C₃-alkylamino-C₁-C₃-alkyl,
R⁸ is hydroxyl, C₁-C₆-alkyl, C₁-C₆-alkoxy, halo C₁-C₃-alkyl hydroxy-C₁-C₃-alkyl, C₁-C₃-alkoxy-C₁-C₃-alkyl C₃-C₈-cycloalkyl, phenyl, monocyclic heterocyclyl having 4 to 8 ring atoms or monocyclic heteroaryl having 5 or 6 ring atoms, where phenyl, heteroaryl and heterocyclyl are optionally mono- or disubstituted by halogen, C₁-C₃-alkoxy or C₁-C₃-alkyl,
R⁹ is C₁-C₆-alkyl,
R¹⁰ and R¹¹ are each independently hydrogen, C₁-C₆-alkyl or a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy C₁-C₆-alkyl hydroxy-C₁-C₆-alkyl, C₁-C₆-alkylamino, C₁-C₆-alkylcarbonylamino, amino C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl C₁-C₆-alkyl phenoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ or a monocyclic heterocyclyl radical having 4 to 8 ring atoms,
or
R¹⁰ and R¹¹ together with the adjacent nitrogen atom are a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, nitro, hydroxyl, amino, oxo, carboxyl, C₁-C₆-alkylamino, C₁-C₆-alkylcarbonylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, phenoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ or a monocyclic heterocyclyl radical having 4 to 8 ring atoms,
R¹² is C₁-C₆-alkyl, halo-C₁-C₃-alkyl, hydroxy-C₁-C₃-alkyl, C₁-C₃-alkoxy-C₁-C₃-alkyl, C₃-C₈-cycloalkyl, phenyl, a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, C₁-C₆-alkylcarbonylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, phenoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ or a monocyclic heterocyclyl radical having 4 to 8 ring atoms,
R¹³ is C₁-C₆-alkyl, halo-C₁-C₃-alkyl, hydroxy-C₁-C₃-alkyl, C₁-C₃-alkoxy-C₁-C₃-alkyl, C₃-C₈-cycloalkyl, phenyl, a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, C₁-C₆-alkylcarbonylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, phenoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ or a monocyclic heterocyclyl radical having 4 to 8 ring atoms,
or an enantiomer, diastereomer, tautomer, solvate, physiologically acceptable salt or solvate of a salt thereof.

3. A compound according to claim 1, in which
X is a carbon or nitrogen atom,
n and m are each independently 0 or 1,
p is 1,
R¹, R⁴ and R⁵ are each independently hydrogen, hydroxyl, cyano, aminocarbonyl, halogen or C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl, wherein the C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl is optionally substituted singly or multiply, identically or differently by halogen, amino, hydroxyl, carboxyl, hydroxy-C₁-C₆-alkyl, C₁-C₆-alkoxy, C₁-C₆-alkoxy-C₁-C₆-alkyl, C₁-C₆-alkylamino or amino-C₁-C₆-alkyl
R² is hydrogen or is C₁-C₆-alkyl, C₁-C₆-alkylcarbonyl, phenylsulphonyl or C₁-C₆-alkylsulphonyl, each optionally mono- or polysubstituted identically or differently by halogen, amino, hydroxyl or carboxyl,
or
is C₃-C₁₀-cycloalkyl optionally mono- or polysubstituted identically or differently by halogen, C₁-C₆-alkyl or C₁-C₆-alkoxy,
or
is phenyl optionally mono- or polysubstituted identically or differently by halogen, hydroxyl, cyano, carboxyl, C₁-C₆-alkyl, C₁-C₆-alkoxy, halo-C₁-C₆-alkoxy, C₃-C₁₀-cycloalkyl or a monocyclic heterocyclyl radical having 4 to 8 ring atoms, and
R³ is hydrogen or is C₁-C₆-alkyl, C₁-C₆-alkylcarbonyl, phenylsulphonyl or C₁-C₆-alkylsulphonyl, each optionally mono- or polysubstituted identically or differently by halogen, amino, hydroxyl, carboxyl, hydroxy-C₁-C₆-alkyl, C₁-C₆-alkoxy, C₁-C₆-alkoxy-C₁-C₆-alkyl, C₁-C₆-alkylamino or amino-C₁-C₆-alkyl, when X is a carbon atom,
or
R³ is hydrogen or is C₁-C₆-alkyl, C₁-C₆-alkylcarbonyl, phenylsulphonyl or C₁-C₆-alkylsulphonyl, each optionally mono- or polysubstituted identically or differently by halogen, amino, hydroxyl, carboxyl, hydroxy-C₁-C₆-alkyl, C₁-C₆-alkoxy, C₁-C₆-alkoxy-C₁-C₆-alkyl, C₁-C₆-alkylamino or amino-C₁-C₆-alkyl, when X is a nitrogen atom,
Y is a —C(═O)OR¹², —C(═O)R¹³ or —C(═O)NR¹⁰R¹¹ group, or is phenyl, monocyclic heterocyclyl having 4 to 8 ring atoms or mono- or bicyclic heteroaryl having 5 to 10 ring atoms, in which phenyl, heteroaryl and heterocyclyl are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, phenoxy, halophenyl, phenyl-C₁-C₆-alkyl, phenyl-C₁-C₆-alkoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, or a monocyclic heterocyclyl radical which has 4 to 8 ring atoms and is itself optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl or halo-C₁-C₆-alkoxy,
R⁶ and R⁷ are each independently hydrogen, C₁-C₃-alkyl, cyclopropyl or di-C₁-C₃-alkylamino-C₁-C₃-alkyl,
R⁸ is hydroxyl, C₁-C₆-alkyl, C₁-C₆-alkoxy, halo-C₁-C₃-alkyl, hydroxy-C₁-C₃-alkyl, C₁-C₃-alkoxy-C₁-C₃-alkyl, C₃-C₈-cycloalkyl, phenyl, monocyclic heterocyclyl having 4 to 8 ring atoms or monocyclic heteroaryl having 5 or 6 ring atoms, where phenyl, heteroaryl and heterocyclyl are optionally mono- or disubstituted by halogen, C₁-C₃-alkoxy or C₁-C₃-alkyl,
R⁹ is C₁-C₆-alkyl,
R¹⁰ and R¹¹ are each independently hydrogen, C₁-C₆-alkyl or a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, C₁-C₆-alkylcarbonylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, phenoxy, pyridinyl, —C(═O)—NR⁶R⁷, —C(═O)—R⁸, —S(═O)₂—NR⁶R⁷, —S(═O)R⁹, —S(O)₂—R⁹, —NH—S(═O)₂—R⁹ or a monocyclic heterocyclyl radical having 4 to 8 ring atoms,
or
R¹⁰ and R¹¹ together with the adjacent nitrogen atom are a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, hydroxyl, amino, oxo, C₁-C₆-alkyl, C₁-C₆-alkoxy, C₁-C₆-alkylamino, C₁-C₆-alkylcarbonylamino, halo-C₁-C₆-alkyl, halo-C₁-C₆-alkoxy, C₃-C₁₀-cycloalkyl, phenyl, halophenyl, phenyl-C₁-C₆-alkyl, phenoxy, —C(═O)—NR⁶R⁷, —C(═O)—R⁸, —S(═O)₂—NR⁶R⁷, —S(═O)₂—R⁹, —NH—S(═O)₂—R⁹ or a monocyclic heterocyclyl radical having 4 to 8 ring atoms,
R¹² is C₁-C₆-alkyl, halo-C₁-C₃-alkyl, hydroxy-C₁-C₃-alkyl, C₁-C₃-alkoxy-C₁-C₃-alkyl, C₃-C₈-cycloalkyl, phenyl, a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, C₁-C₆-alkylcarbonylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, phenoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ or a monocyclic heterocyclyl radical having 4 to 8 ring atoms,
R¹³ is C₁-C₆-alkyl, halo-C₁-C₃-alkyl, hydroxy-C₁-C₃-alkyl, C₁-C₃-alkoxy-C₁-C₃-alkyl, C₃-C₈-cycloalkyl, phenyl, a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, each optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, C₁-C₆-alkylcarbonylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, phenoxy, pyridinyl, —C(═O)—NR⁶R⁷, C(═O)—R⁸, —S(═O)₂—NR⁶R⁷, —S(═O)—R⁹, —S(═O)₂—R⁹, —NH—S(═O)₂—R⁹ or a monocyclic heterocyclyl radical having 4 to 8 ring atoms,
or an enantiomer, diastereomer, tautomer, solvate, physiologically acceptable salt or solvate of a salt thereof.

4. A compound according to claim 1 in which
X is a carbon or nitrogen atom,
n and m are each independently 0 or 1,
p is 1,
R¹, R⁴ and R⁵ are each independently hydrogen or halogen,
R² is hydrogen or C₁-C₆-alkyl,
R³ is C₁-C₆-alkyl when X is a carbon atom,
or
R³ is hydrogen or C₁-C₆-alkyl when X is a nitrogen atom, Y is a —C(═O)OR¹² or —C(═O)NR¹⁰R¹¹ group, or is phenyl, monocyclic heterocyclyl having 4 to 8 ring atoms or monocyclic heteroaryl having 5 or 6 ring atoms, in which phenyl, heteroaryl and heterocyclyl are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, phenoxy, halophenyl, phenyl-C₁-C₆-alkyl, phenyl-C₁-C₆-alkoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, or a monocyclic heterocyclyl radical which has 4 to 8 ring atoms and is itself optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl or halo-C₁-C₆-alkoxy,
R⁶ and R⁷ are each independently hydrogen, C₁-C₃-alkyl, cyclopropyl or di-C₁-C₃-alkylamino-C₁-C₃-alkyl,
R⁸ is hydroxyl, C₁-C₆-alkyl, C₁-C₆-alkoxy, halo-C₁-C₃-alkyl, hydroxy-C₁-C₃-alkyl, C₁-C₃-alkoxy-C₁-C₃-alkyl, C₃-C₈-cycloalkyl, phenyl, monocyclic heterocyclyl having 4 to 8 ring atoms or monocyclic heteroaryl having 5 or 6 ring atoms, where phenyl, heteroaryl and heterocyclyl are optionally mono- or disubstituted by halogen, C₁-C₃-alkoxy or C₁-C₃-alkyl,
R⁹ is C₁-C₆-alkyl,
R¹⁰ and R¹¹ are each independently hydrogen, C₁-C₆-alkyl or a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, C₁-C₆-alkylcarbonylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, phenoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ or a monocyclic heterocyclyl radical having 4 to 8 ring atoms,
or
R¹⁰ and R¹¹ together with the adjacent nitrogen atom are a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, hydroxyl, amino, oxo, C₁-C₆-alkyl, C₁-C₆-alkoxy, C₁-C₆-alkylamino, C₁-C₆-alkylcarbonylamino, halo-C₁-C₆-alkyl, halo-C₁-C₆-alkoxy, C₃-C₁₀-cycloalkyl, phenyl, halophenyl, phenyl-C₁-C₆-alkyl, phenoxy, —C(═O)—NR⁶R⁷, —C(═O)—R⁸, —S(═O)₂—NR⁶R⁷, —S(═O)₂—R⁹, —NH—S(═O)₂—R⁹ or a monocyclic heterocyclyl radical having 4 to 8 ring atoms,
R¹² is C₁-C₆-alkyl, halo-C₁-C₃-alkyl, hydroxy-C₁-C₃-alkyl, C₁-C₃-alkoxy-C₁-C₃-alkyl, C₃-C₈-cycloalkyl, phenyl, a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, C₁-C₆-alkylcarbonylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, phenoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ or a monocyclic heterocyclyl radical having 4 to 8 ring atoms,
or an enantiomer, diastereomer, tautomer, solvate, physiologically acceptable salt or solvate of a salt thereof.

5. A compound according to claim 1 in which
X is a carbon or nitrogen atom,
n and m are each independently 0 or 1,
p is 1,
R¹, R⁴ and R⁵ are each independently hydrogen or halogen,
R² is hydrogen or C₁-C₆-alkyl,
R³ is C₁-C₆-alkyl when X is a carbon atom,
or
R³ is hydrogen or C₁-C₆-alkyl when X is a nitrogen atom,
Y is a —C(═O)OR¹² or —C(═O)NR¹⁰R¹¹ group, or is monocyclic heteroaryl which has 5 or 6 ring atoms and is optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, phenoxy, halophenyl, phenyl-C₁-C₆-alkyl, phenyl-C₁-C₆-alkoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, or a monocyclic heterocyclyl radical which has 4 to 8 ring atoms and is itself optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl or halo-C₁-C₆-alkoxy,
R⁶ and R⁷ are each independently hydrogen, C₁-C₃-alkyl, cyclopropyl or di-C₁-C₃-alkylamino-C₁-C₃-alkyl,
R⁸ is hydroxyl, C₁-C₆-alkyl, C₁-C₆-alkoxy, halo-C₁-C₃-alkyl, hydroxy-C₁-C₃-alkyl, C₁-C₃-alkoxy-C₁-C₃-alkyl, C₃-C₈-cycloalkyl, phenyl, monocyclic heterocyclyl having 4 to 8 ring atoms or monocyclic heteroaryl having 5 or 6 ring atoms, where phenyl, heteroaryl and heterocyclyl are optionally mono- or disubstituted by halogen, C₁-C₃-alkoxy or C₁-C₃-alkyl,
R⁹ is C₁-C₆-alkyl,
R¹⁰ and R¹¹ are each independently hydrogen, C₁-C₆-alkyl or a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, nitro, hydroxyl, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, C₁-C₆-alkylcarbonylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, phenoxy, pyridinyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ or a monocyclic heterocyclyl radical having 4 to 8 ring atoms,
or
R¹⁰ and R¹¹ together with the adjacent nitrogen atom are a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by halogen, cyano, hydroxyl, amino, oxo, C₁-C₆-alkyl, C₁-C₆-alkoxy, C₁-C₆-alkylamino, C₁-C₆-alkylcarbonylamino, halo-C₁-C₆-alkyl, halo-C₁-C₆-alkoxy, C₃-C₁₀-cycloalkyl, phenyl, halophenyl, phenyl-C₁-C₆-alkyl, phenoxy, —C(═O)—NR⁶R⁷, —C(═O)—R⁸, —S(═O)₂—NR⁶R⁷, —S(═O)₂—R⁹, —NH—S(═O)₂—R⁹ or a monocyclic heterocyclyl radical having 4 to 8 ring atoms,
R¹² is C₁-C₆-alkyl, C₃-C₈-cycloalkyl or a monocyclic or bicyclic heterocyclyl radical having 4 to 12 ring atoms, where the radicals mentioned are optionally mono- or polysubstituted identically or differently by halogen, oxo, C₁-C₆-alkyl or phenoxy,
or an enantiomer, diastereomer, tautomer, solvate, physiologically acceptable salt or solvate of a salt thereof.

6. A compound according to claim 1 in which
X is a carbon or nitrogen atom,
n and m are each independently 0 or 1,
p is 1,
R¹, R⁴ and R⁵ are each independently hydrogen or halogen,
is hydrogen or C₁-C₆-alkyl,
R³ is C₁-C₆-alkyl when X is a carbon atom,
or
R³ is hydrogen or C₁-C₆-alkyl when X is a nitrogen atom,
Y is a —C(═O)OR¹² or —C(═O)NR¹⁰R¹¹ group, or is monocyclic heteroaryl which has 5 ring atoms and is optionally mono- or polysubstituted by C₁-C₆-alkyl or C₃-C₁₀-cycloalkyl,
R⁶ and R⁷ are each independently hydrogen, C₁-C₃-alkyl, cyclopropyl or di-C₁-C₃-alkylamino-C₁-C₃-alkyl,
R⁸ is hydroxyl, C₁-C₆-alkyl, C₁-C₆-alkoxy, halo-C₁-C₃-alkyl, hydroxy-C₁-C₃-alkyl, C₁-C₃-alkoxy-C₁-C₃-alkyl, C₃-C₈-cycloalkyl, phenyl, monocyclic heterocyclyl having 4 to 8 ring atoms or monocyclic heteroaryl having 5 or 6 ring atoms, where phenyl, heteroaryl and heterocyclyl are optionally mono- or disubstituted by halogen, C₁-C₃-alkoxy or C₁-C₃-alkyl,
R⁹ is C₁-C₆-alkyl,
R¹⁰ and R¹¹ are each independently hydrogen, C₁-C₆-alkyl or a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by oxo or —C(═O)—R⁸,
or
R¹⁰ and R¹¹ together with the adjacent nitrogen atom are a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned may optionally include 1, 2 or 3 heteroatoms from the group of nitrogen, oxygen and sulphur, and are optionally mono- or polysubstituted identically or differently by halogen, cyano, amino, oxo, C₁-C₆-alkyl, C₁-C₆-alkoxy, C₁-C₆-alkylamino, C₁-C₆-alkylcarbonylamino, halo-C₁-C₆-alkyl, halo-C₁-C₆-alkoxy, C₃-C₁₀-cycloalkyl, phenyl, halophenyl, phenoxy, —C(═O)—NR⁶R⁷, —C(═O)—R⁸, —S(═O)₂—NR⁶R⁷, —S(═O)₂—R⁹ or NH—S(═O)₂—R⁹,
R¹² is C₁-C₆-alkyl, C₃-C₈-cycloalkyl or a monocyclic or bicyclic heterocyclyl radical having 4 to 12 ring atoms, where the radicals mentioned are optionally mono- or polysubstituted identically or differently by halogen, oxo, C₁-C₆-alkyl or phenoxy,
or an enantiomer, diastereomer, tautomer, solvate, physiologically acceptable salt or solvate of a salt thereof.

7. A compound according to claim 1 in which
X is a carbon or nitrogen atom,
n and m are each independently 0 or 1,
p is 1,
R¹ is hydrogen or halogen,
R² is hydrogen or C₁-C₆-alkyl,
R³ is C₁-C₆-alkyl when X is a carbon atom,
or
R³ is hydrogen or C₁-C₆-alkyl when X is a nitrogen atom,
R⁴ and R⁵ are hydrogen,
Y is a —C(═O)OR¹² or —C(═O)NR¹⁰R¹¹ group, or is monocyclic heteroaryl which has 5 ring atoms and is optionally mono- or polysubstituted by C₁-C₆-alkyl or C₃-C₁₀-cycloalkyl,
R⁸ is C₁-C₆-alkoxy,
R¹⁰ and R¹¹ are each independently hydrogen, C₁-C₆-alkyl or a monocyclic or bicyclic heterocyclyl radical having 4 to 12 ring atoms or a monocyclic or bicyclic heteroaryl radical having 5 to 10 ring atoms or a partly saturated bicyclic aryl or heteroaryl radical having 7 to 11 ring atoms, where the radicals mentioned are optionally mono- or polysubstituted identically or differently by oxo or —C(═O)—R⁸,
or
R¹⁰ and R¹¹ together with the adjacent nitrogen atom are a monocyclic or bicyclic heterocyclyl radical which has 4 to 12 ring atoms and is optionally mono- or polysubstituted identically or differently by halogen, oxo, phenoxy or —C(═O)—R⁸,
R¹² is C₁-C₆-alkyl, C₃-C₈-cycloalkyl or a monocyclic or bicyclic heterocyclyl radical having 4 to 12 ring atoms, where the radicals mentioned are optionally mono- or polysubstituted identically or differently by halogen, oxo, C₁-C₆-alkyl or phenoxy,
or an enantiomer, diastereomer, tautomer, solvate, physiologically acceptable salt or solvate of a salt thereof.

8. A compound according to claim 1 in which
X is a carbon or nitrogen atom,
n and m are each independently 0 or 1,
p is 1,
R¹ is hydrogen or chlorine,
R² is hydrogen or methyl,
R³ is methyl when X is a carbon atom,
or
R³ is hydrogen or methyl when X is a nitrogen atom,
R⁴ and R⁵ are hydrogen,
Y is a —C(═O)OR¹² or —C(═O)NR¹⁰R¹¹ group or a monocyclic heteroaryl radical having 5 ring atoms of the structure: 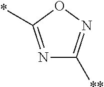
which may be substituted at “**” by methyl, ethyl, isopropyl, tert-butyl or cyclopropyl, and in which “*” denotes the attachment point to the rest of the molecule,
R¹⁰ is hydrogen or methyl,
R¹¹ is ethyl or one of the following groups: 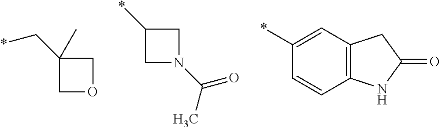
where “*” denotes the attachment point to the nitrogen atom in the —C(═O)NR¹⁰R¹¹ group defined in Y,
or
R¹⁰ and R¹¹ together with the adjacent nitrogen atom are one of the following groups: 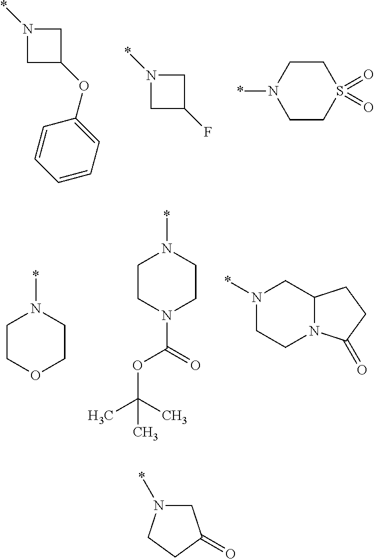
where “*” denotes the attachment point to the carbonyl group in the —C(═O)NR¹⁰R¹¹ group defined in Y,
R¹² is methyl or tert-butyl,
or an enantiomer, diastereomer, tautomer, solvate, physiologically acceptable salt or solvate of a salt thereof.

9. A compound according to claim 1 selected from the group consisting of:
2-(4S)-[6-(4-chlorophenyl)-1,7,8-trimethyl-4,8-dihydropyrrolo[3,4-f][1,2,4]triazolo[4,3-a][1,4]diazepin-4-yl]acetic acid methyl ester;
2-(4S)-(1,7,8-trimethyl-6-phenyl-4,8-dihydropyrrolo[3,4-f][1,2,4]triazolo[4,3-a][1,4]diazepin-4-yl)acetic acid methyl ester;
(−)-2-(4S)-[6-(4-chlorophenyl)-1,7,8-trimethyl-4,8-dihydropyrrolo[3,4-f][1,2,4]triazolo[4,3-a][1,4]diazepin-4-yl]acetic acid tert-butyl ester;
(−)-2-(4S)-(1,7,8-trimethyl-6-phenyl-4,8-dihydropyrrolo[3,4-f][1,2,4]triazolo[4,3-a][1,4]diazepin-4-yl)acetic acid tert-butyl ester;
(−)-2-[(4S)-6-(4-chlorophenyl)-1,7,8-trimethyl-4,8-dihydropyrrolo[3,4-f][1,2,4]triazolo[4,3-a][1,4]diazepin-4-yl]-1-(3-fluoroazetidin-1-yl)ethan-1-one;
2-[(4S)-6-(4-chlorophenyl)-1,8-dimethyl-4,8-dihydropyrazolo[3,4-f][1,2,4]triazolo[4,3-a][1,4]diazepin-4-yl]acetic acid methyl ester;
2-[(4S)-6-(4-chlorophenyl)-1,7-dimethyl-4,7-dihydropyrazolo[3,4-f][1,2,4]triazolo[4,3-a][1,4]diazepin-4-yl]acetic acid methyl ester;
(−)-2-[(4S)-6-(4-chlorophenyl)-1,7,8-trimethyl-4,8-dihydropyrrolo[3,4-f][1,2,4]triazolo[4,3-a][1,4]diazepin-4-yl]-1-(1,1-dioxidothiomorpholino)ethan-1-one;
N-(1-acetylazetidin-3-yl)-2-(4S)-[6-(4-chlorophenyl)-1,7,8-trimethyl-4,8-dihydropyrrolo[3,4-f][1,2,4]triazolo[4,3-a][1,4]diazepin-4-yl]acetamide;
(−)-2-[(4S)-6-(4-chlorophenyl)-1,7,8-trimethyl-4,8-dihydropyrrolo[3,4-f][1,2,4]triazolo[4,3-a][1,4]diazepin-4-yl]-N-ethylacetamide;
(−)-2-[(4S)-6-(4-chlorophenyl)-1,7,8-trimethyl-4,8-dihydropyrrolo[3,4-f][1,2,4]triazolo[4,3-a][1,4]diazepin-4-yl]-N-methyl-N-[(3-methyloxetan-3-yl)methyl]acetamide;
(−)-1-{[(4S)-6-(4-chlorophenyl)-1,7,8-trimethyl-4,8-dihydropyrrolo[3,4-f][1,2,4]triazolo[4,3-a][1,4]diazepin-4-yl]acetyl}pyrrolidin-3-one;
2-[(4S)-6-(4-chlorophenyl)-1,7,8-trimethyl-4,8-dihydropyrrolo[3,4-f][1,2,4]triazolo[4,3-a][1,4]diazepin-4-yl]-N-(2-oxo-2,3-dihydro-1H-indol-5-yl)acetamide;
2-[6-(4-chlorophenyl)-1,8-dimethyl-4,8-dihydropyrazolo[3,4-f][1,2,4]triazolo[4,3-a][1,4]diazepin-4-yl]-1-(1,1-dioxidothiomorpholin-4-yl)ethanone;
2-[(4S)-6-(4-chlorophenyl)-1,8-dimethyl-4,8-dihydropyrazolo[3,4-f][1,2,4]triazolo[4,3-a][1,4]diazepin-4-yl]-1-(1,1-dioxidothiomorpholin-4-yl)ethanone;
2-[6-(4-chlorophenyl)-1,8-dimethyl-4,8-dihydropyrazolo[3,4-f][1,2,4]triazolo[4,3-a][1,4]diazepin-4-yl]-1-(morpholin-4-yl)ethanone;
2-[6-(4-chlorophenyl)-1,8-dimethyl-4,8-dihydropyrazolo[3,4-f][1,2,4]triazolo[4,3-a][1,4]diazepin-4-yl]-1-(3-fluoroazetidin-1-yl)ethanone;
2-[6-(4-chlorophenyl)-1,8-dimethyl-4,8-dihydropyrazolo[3,4-f][1,2,4]triazolo[4,3-a][1,4]diazepin-4-yl]-N-ethylacetamide;
1-(morpholin-4-yl)-2-[(4S)-1,7,8-trimethyl-6-phenyl-4,8-dihydropyrrolo[3,4-f][1,2,4]triazolo[4,3-a][1,4]diazepin-4-yl]ethanone;
(−)-2-[(4S)-6-(4-chlorophenyl)-1,7,8-trimethyl-4,8-dihydropyrrolo[3,4-f][1,2,4]triazolo[4,3-a][1,4]diazepin-4-yl]-1-(3-phenoxyazetidin-1-yl)ethanone;
(−)-2-[(4S)-6-(4-chlorophenyl)-1,7,8-trimethyl-4,8-dihydropyrrolo[3,4-f][1,2,4]triazolo[4,3-a][1,4]diazepin-4-yl]-1-(morpholin-4-yl)ethanone;
(−)-4-{[(4S)-6-(4-chlorophenyl)-1,7,8-trimethyl-4,8-dihydropyrrolo[3,4-f][1,2,4]triazolo[4,3-a][1,4]diazepin-4-yl]acetyl}piperazine-1-carboxylic acid tert-butyl ester;
(−)-2-{[(4S)-6-(4-chlorophenyl)-1,7,8-trimethyl-4,8-dihydropyrrolo[3,4-f][1,2,4]triazolo[4,3-a][1,4]diazepin-4-yl]acetyl}hexahydropyrrolo[1,2-a]pyrazin-6(2H)-one;
6-(4-chlorophenyl)-4-[(3-cyclopropyl-1,2,4-oxadiazol-5-yl)methyl]-1,7,8-trimethyl-4,8-dihydropyrrolo[3,4-f][1,2,4]triazolo[4,3-a][1,4]diazepine;
6-(4-chlorophenyl)-1,7,8-trimethyl-4-[(3-methyl-1,2,4-oxadiazol-5-yl)methyl]-4,8-dihydropyrrolo[3,4-f][1,2,4]triazolo[4,3-a][1,4]diazepine;
(4S)-6-(4-chlorophenyl)-1,7,8-trimethyl-4-[(3-methyl-1,2,4-oxadiazol-5-yl)methyl]-4,8-dihydropyrrolo[3,4-f][1,2,4]triazolo[4,3-a][1,4]diazepine;
6-(4-chlorophenyl)-1,7,8-trimethyl-4-[(3-ethyl-1,2,4-oxadiazol-5-yl)methyl]-4,8-dihydropyrrolo[3,4-f][1,2,4]triazolo[4,3-a][1,4]diazepine;
(4S)-6-(4-chlorophenyl)-1,7,8-trimethyl-4-[(3-ethyl-1,2,4-oxadiazol-5-yl)methyl]-4,8-dihydropyrrolo[3,4-f][1,2,4]triazolo[4,3-a][1,4]diazepine;
6-(4-chlorophenyl)-1,7,8-trimethyl-4-{[3-(propan-2-yl)-1,2,4-oxadiazol-5-yl]methyl}-4,8-dihydropyrrolo[3,4-f][1,2,4]triazolo[4,3-a][1,4]diazepine;
(4S)-6-(4-chlorophenyl)-1,7,8-trimethyl-4-{[3-(propan-2-yl)-1,2,4-oxadiazol-5-yl]methyl}-4,8-dihydropyrrolo[3,4-f][1,2,4]triazolo[4,3-a][1,4]diazepine and
4-[(3-tert-butyl-1,2,4-oxadiazol-5-yl)methyl]-6-(4-chlorophenyl)-1,7,8-trimethyl-4,8-dihydropyrrolo[3,4-f][1,2,4]triazolo[4,3-a][1,4]diazepine.

10. (canceled)

11. (canceled)

12. A method of controlling male fertility comprising administering to a patient in need thereof an effective amount of a compound according to claim 1.

13. A method of the treatment of leukaemias, prostate carcinomas, mammary carcinomas, melanomas or multiple myelomas comprising administering to a patient in need thereof a therapeutically effective amount of a compound according to claim 1.

14. (canceled)

15. (canceled)

16. A pharmaceutical composition comprising a compound according to claim 1 in combination with a further active ingredient.

17. A pharmaceutical formulation comprising a compound according to claim 1 and a pharmaceutically acceptable excipient.
